(19) United States
(12) Patent Application Publication
(10) Pub. No.: US 2023/0097018 A1
(43) Pub. Date: Mar. 30, 2023

(54) KINETIC LEARNING

(71) Applicant: The Regents of the University of California, Oakland, CA (US)

(72) Inventors: Zachary Costello, Berkeley, CA (US); Hector Garcia Martin, Oakland, CA (US)

(21) Appl. No.: 17/948,911

(22) Filed: Sep. 20, 2022

Related U.S. Application Data
(60) Provisional application No. 63/246,114, filed on Sep. 20, 2021.

(51) Int. Cl. G16B 40/00; G16B 20/00; G16B 45/00

(52) U.S. Cl. CPC G16B 40/00; G16B 20/00; G16B 45/00

(57) ABSTRACT

Disclosed herein include systems, devices, and methods for kinetic learning, which can include, for example, training and/or using a machine learning model, such as training a machine learning model and using the machine learning model to simulate a virtual strain of an organism or to determine possible modifications of an organism.

### RELATED APPLICATIONS

This application claims the benefit under 35 U.S.C. § 119(e) of U.S. Provisional Patent Application No. 63/246,114, filed Sep. 20, 2021, the content of which is incorporated herein by reference in its entirety for all purposes.

### STATEMENT REGARDING FEDERALLY SPONSORED R&D

This invention was made with government support under grant no. DE-ACO2-05CH11231 awarded by the U.S. Department of Energy. The government has certain rights in the invention.

### BACKGROUND

### Field

The present disclosure relates generally to the field of computational biology, and more particularly to determining dynamics of metabolic pathways.

### Description of the Related Art

New synthetic biology capabilities hold the promise of dramatically improving our ability to engineer biological systems. However, a fundamental hurdle in realizing this potential is the inability to accurately predict biological behavior after modifying the corresponding genotype. Kinetic models have traditionally been used to predict pathway dynamics in bioengineered systems, but they take significant time to develop, and rely heavily on domain expertise. There is a need for methods that can effectively predict pathway dynamics in an automated fashion.

### SUMMARY

Disclosed herein include methods for simulating a virtual strain of an organism. In some embodiments, a method for simulating a virtual strain of an organism comprises receiving time-series multiomics data of an organism, wherein the times-series multiomics data comprises time-series proteomics data of a plurality of proteins and time-series metabolomics data comprising a characteristic of a metabolite. The method can comprise training a machine learning model with time-series proteomics data as input and the time-series metabolomics data of the metabolite as output. The method can comprise simulating a virtual strain of the organism using the machine learning model to determine the characteristic of the metabolite in the virtual strain.

In some embodiments, the time-series multiomics data comprises time-series multiomics data of a plurality of strains of the organism. In some embodiments, the time-series proteomics data is associated with a metabolic pathway. In some embodiments, wherein the metabolic pathway comprises a heterologous pathway. In some embodiments, the machine learning model represents kinetics of the metabolic pathway.

In some embodiments, the characteristic of the metabolite is a titer, rate, concentration, or yield of the metabolite. In some embodiments, the proteomics data comprises a concentration of each of a plurality of proteins at each of a plurality of time points, and wherein the metabolomics data comprises a concentration of the metabolite at each of the plurality of time points. In some embodiments, the multiomics data comprises triplicates of a concentration of a protein at a time point and triplicates of a concentration of the metabolite at a time point. In some embodiments, simulating the virtual strain of the organism comprises determining a concentration of the metabolite of the virtual strain using the machine learning model.

In some embodiments, the machine learning model comprises a supervised machine learning model. In some embodiments, the machine learning model comprises a non-classification model, a neural network, a recurrent neural network (RNN), a linear regression model, a logistic regression model, a decision tree, a support vector machine, a Naïve Bayes network, a k-nearest neighbors (KNN) model, a k-means model, a random forest model, a multilayer perceptron, or a combination thereof. In some embodiments, the machine learning model comprises a deep neural network (DNN), deep recurrent neural network (DRNN), gated recurrent unit (GRU) DRNN, a partial least square (PLS) model, or a combination thereof. In some embodiments, the machine learning model comprises an ensemble model of a plurality of machine learning models, optionally wherein the plurality of machine learning models comprises a deep neural network (DNN), deep recurrent neural network (DRNN), and gated recurrent unit (GRU) DRNN.

In some embodiments, the virtual strain comprises an increased expression of at least one first protein, a knock-out of at least one second protein, a reduced expression of at least one third protein, or a combination thereof. In some embodiments, the at least one first protein comprises at least 2, 3, 4, 5, 6, 7, 8, 9, 10, 20, 30, 40, 50, or more, first proteins. In some embodiments, the at least one second protein comprises at least 2, 3, 4, 5, 6, 7, 8, 9, 10, 20, 30, 40, 50, or more, second proteins. In some embodiments, the at least one third protein comprises at least 2, 3, 4, 5, 6, 7, 8, 9, 10, 20, 30, 40, 50, or more, third proteins.

In some embodiments, the method comprises designing one or more new strains based on the virtual strain. The method can comprise receiving experimental time-series multiomics data for the new strains. The method can comprise retraining the machine learning model based on the experimental time-series multiomics data of the new strains.

In some embodiments, the method comprise interpolating the time-series multiomics data from a first number of time points to a second number of time points. In some embodiments, the first number of time points comprises, 4, 5, 6, 7, 8, 9, 10, 20, 30, 40, 50, or more, time points. In some embodiments, the second number of time points comprises 50, 55, 56, 57, 58, 59, 60, 61, 62, 63, 64, 65, 70, 75, 80, or more, time points. The first number of time points can be hourly time points. The second number of time points can be hourly time points. Interpolating the time-series multiomics can data comprise interpolating the time-series multiomics data using a cubic spline method.

In some embodiments, a method of stimulating a strain of an organism comprises receiving time-series multiomics data of a plurality of strains of an organism comprising time-series proteomics data of a plurality of proteins and time-series metabolomics data comprising a characteristic of a metabolite. The method can comprise training a machine learning model with the time-series proteomics data as input and the time-series metabolomics data of the metabolite as output. The method can comprise simulating a virtual strain of the organism using the machine learning model to determine the characteristic of the metabolite in the virtual strain.

In some embodiments, receiving the time-series multiomics data comprises data checking and/or preprocessing of the time-series multiomics data of the plurality of strains of the organism.

In some embodiments, the time-series multiomics data comprises multiomics data of two or more time-series of a strain, such as 3, 4, 5, 6, 7, 8, 9, 10, 20, 30, 40, 50, 100, or more. In some embodiments, the time-series multiomics data comprises time-series proteomics data, time-series metabolomics data, time-series transcriptomics data, or a combination thereof. In some embodiments, the multiomics data comprises observations of each of a plurality of proteins at a plurality of time points and observations of the metabolite at the plurality of time points.

In some embodiments, the machine learning model comprises a supervised machine learning model. In some embodiments, machine learning model comprises a deep neural network (DNN), deep recurrent neural network (DRNN), gated recurrent unit (GRU) DRNN, a partial least square (PLS) model, or a combination thereof. In some embodiments, the machine learning model comprises an ensemble model of a plurality of machine learning models, optionally wherein the plurality of machine learning models comprises a deep neural network (DNN), deep recurrent neural network (DRNN), and gated recurrent unit (GRU) DRNN.

In some embodiments, simulating the virtual strain of the organism comprises simulating the virtual strain of the organism using the machine learning model to change one or more of titer, rate, concentration, and yield of the metabolite.

In some embodiments, the method comprises comprising designing a strain of the organism corresponding to the virtual strain. In some embodiments, the method comprises creating a strain of the organism corresponding to the virtual strain.

Disclosed herein include methods for determining modifications of protein expression an organism. In some embodiments, a method for determining modifications of protein expression of an organism comprises: receiving time-series multiomics data of a plurality of strains of an organism comprising time-series proteomics data of comprising a characteristic of each of a plurality of proteins and time-series metabolomics data comprising a characteristic of a metabolite. The method can comprise training a machine learning model with the time-series proteomics data as input and the time-series metabolomics data of the metabolite as output. The method can comprise determining modifications of a concentration of each of one or more proteins using the machine learning model.

In some embodiments, the characteristic of each of the plurality of proteins comprises a concentration of the protein, and/or wherein the characteristic of the metabolite comprises a concentration of the metabolite. In some embodiments, the modifications comprise an increased expression of at least one first protein, a knock-out of at least one second protein, a reduced expression of at least one third protein, or a combination thereof, optionally wherein the at least one first protein comprises at least 10 first proteins, optionally wherein the at least one second protein comprises at least 10 second proteins, optionally wherein the at least one third protein comprises at least 10 third proteins.

Disclosed herein include systems for simulating the pathway dynamics of a virtual strain of an organism. In some embodiments, a system for simulating the pathway dynamics of a virtual strain comprises computer-readable memory storing executable instructions; and one or more hardware processors. The hardware processors can be programmed by the executable instructions to perform: receiving time-series multiomics data of a plurality of strains of the organism, the times-series multiomics data comprising time-series metabolomics data and time-series proteomics data associated with a metabolic pathway. The hardware processors can be programmed by the executable instructions to perform: determining derivatives of the time-series metabolomics data. The hardware processors can be programmed by the executable instructions to perform: training a machine learning model, representing a metabolic pathway dynamics model, using the time-series multiomics data and the derivatives of the time-series metabolomics data, wherein the metabolic pathway dynamics model relates the time-series metabolomics data and time-series proteomics data to the derivatives of the time-series metabolomics data. The hardware processors can be programmed by the executable instructions to perform: simulating a virtual strain of the organism using the metabolic pathway dynamics model to determine a characteristics of a metabolic pathway represented by the metabolic pathway dynamics model in the virtual strain.

The hardware processors can be programmed by the executable instructions to perform: designing one or more new strains based on the virtual strain; generating experimental time-series multiomics data for the new strains; and retraining the machine learning model based on the experimental time-series multiomics data of the new strains.

The characteristic of the metabolic pathway can be a titer, rate, or yield of a product of the metabolic pathway. The time-series multiomics data can comprise time-series multiomics data of a plurality of strains of an organism. The metabolic pathway can comprise a heterologous pathway.

The machine learning model comprises a supervised machine learning model. The machine learning model can comprise a non-classification model, a neural network, a recurrent neural network (RNN), a linear regression model, a logistic regression model, a decision tree, a support vector machine, a Naïve Bayes network, a k-nearest neighbors (KNN) model, a k-means model, a random forest model, a multilayer perceptron, or a combination thereof. The machine learning model can comprise parameters representing kinetics of the metabolic pathway and parameters associated with the plurality of strains.

Training the machine learning model can comprises training the machine learning model using training data comprising triplets of a protein concentration, a metabolite concentration, and a metabolite derivative. Simulating the virtual strain of the organism can comprise integrating the metabolic pathway dynamics model over a time period of interest. Simulating the virtual strain of the organism can comprise determining a concentration of a metabolite of the metabolic pathway using the metabolic pathway dynamics model.

The one or more hardware processor can be programmed by the executable instructions to perform: smooth the time-series metabolomics data to generate smoothed time-series metabolomics data, wherein determining the derivatives of the time-series metabolomics data comprises determining derivatives of the smoothed time-series metabolomics data, and wherein training the machine learning model comprises training the machine learning model using the smooth time-series multiomics data and the derivatives of the smoothed metabolomics data. Smoothing the time-series metabolomics data can comprise smoothing the time-series metabolomics data using a filter. The filter can comprise a Savitzky-Golay filter.

Disclosed herein include methods for simulating the metabolic pathway dynamics of a strain of an organism. In some embodiments, a method for simulating the metabolic pathway dynamics of a strain of an organism, comprises: receiving time-series multiomics data comprising a first time-series multiomics data associated a metabolic pathway and a second time-series multiomics data associated with the metabolic pathway. The method can comprise: determining derivatives of the first time-series multiomics data. The method can comprise: training a machine learning model, representing a metabolic pathway dynamics model, using the first time-series multiomics data, the derivatives of the first time-series multiomics data, and the second time-series multiomics data, wherein the metabolic pathway dynamics model relates the first time-series multiomics data and the second time-series multiomics data to the derivatives of the first time-series multiomics data. The method can comprise: simulating a virtual strain of the organism using the metabolic pathway dynamics model.

In some embodiments, the first time-series multiomics data comprises time-series metabolomics data of a plurality of strains of an organism, wherein the time-series metabolomics data comprises two or more time-series of a strain. The second time-series multiomics data can comprise time-series proteomics data of a plurality of strains of an organism, and wherein the time-series proteomics data comprises a plurality of time-series of a strain. The first time-series multiomics data can comprise time-series multiomics data of a plurality of strains of an organism, and wherein the first time-series multiomics data comprises time-series multiomics data of a plurality of strains of a different organism.

The first time-series multiomics data or the second time-series multiomics data comprises time-series proteomics data, time-series metabolomics data, time-series transcriptomics data, or a combination thereof. The first time-series multiomics data or the second time-series multiomics data can be associated with an enzymatic characteristic selected from the group consisting of a k_{cat} constant, a Kₘ constant, and a kinetic characteristics curve. The first time-series multiomics data and the second time-series multiomics data can comprise observations at corresponding time points.

The machine learning model can comprise a supervised machine learning model. The machine learning model can comprises observable and unobservable parameters representing kinetics of the metabolic pathway.

Training the machine learning model can comprise training the machine learning model using training data comprising an n-tuples of a first observation at a time point in the first time-series multiomics data, a second observation at the time point in the second time-series multiomics data, and a derivative of the first observation. Training the machine learning model can comprise selecting the machine learning model from a plurality of machine learning models using a tree-based pipeline optimization tool.

Simulating the virtual strain of the organism can comprise integrating derivatives of the first time-series multiomics data outputted by the metabolic pathway dynamics model. Simulating a virtual strain of the organism using the metabolic pathway dynamics model can comprise simulating a virtual strain using the metabolic pathway dynamics model to change one or more of titer, rate, and yield of a product of a metabolic pathway represented by the metabolic pathway dynamics.

The method can comprise designing a strain of the organism corresponding to the simulated strain. The method can comprise creating a strain of the organism corresponding to the simulated strain.

### BRIEF DESCRIPTION OF THE DRAWINGS

FIG. 1 depicts an exemplary flow diagram of kinetic modeling.

FIG. 2 depicts an exemplary workflow of kinetic modeling incorporating machine learning.

FIG. 3 depicts non-limiting exemplary embodiments showing machine learning can be used to relearn Michaeles-Menten more accurately.

FIG. 4 depicts exemplary predictions of isopentenol concentrations.

FIG. 5A-FIG. 5C depict exemplary data showing results of predictions for additional metabolites.

FIG. 6 depicts exemplary embodiments showing that predictions improve substantially as data are added.

FIG. 7 depicts a structure and some exemplary uses of malonic acid.

FIG. 8 depicts a non-limiting exemplary Design, Build, Test, Learn (DBTL) cycle.

FIG. 9 depicts non-limiting exemplary embodiments of 6 DBTL cycles.

FIG. 10 depicts exemplary visualization of multi-omics series from 80,000 data points per DBTL cycle.

FIG. 11 depicts an exemplary workflow for the Experiment Data Depot (EDD) tool.

FIG. 12 depicts non-limiting exemplary embodiments showing the EDD can store data in a standardized manner.

FIG. 13A-FIG. 13K depict non-limiting exemplary embodiments of the functionality of the EDD tool.

FIG. 14A-FIG. 14C depict non-limiting examples for downloading data into, e.g., a Jupyter Notebook, through the Representational State Transfer (REST) application programming interface (API).

FIG. 15 shows non-limiting exemplary data related to model fitting requiring smooth time series.

FIG. 16 depicts non-limiting exemplary embodiments for predicting response timelines from input timelines, rather than derivatives. DNN, Deep Neural Network; DRNN, Deep Recurrent Neural Network; GRU DRNN, Gated Recurrent Unit DRNN; PLS, Partial Least Squares.

FIG. 17 depicts non-limiting exemplary embodiments for predicting response timelines from input timelines. DNN, Deep Neural Network; DRNN, Deep Recurrent Neural Network; GRU DRNN, Gated Recurrent Unit DRNN; PLS, Partial Least Squares.

FIG. 18 depicts non-limiting exemplary data showing that the ensemble model can predict product dynamics. Numbers on the graphs are Pearson r coefficients.

FIG. 19 depicts non-limiting exemplary data showing correlations of predicted vs. observed total malonic acid formed (TMAF). Numbers on the graphs are Pearson r coefficients and mean absolute error (MAE).

FIG. 20 depicts non-limiting exemplary data showing that the ensemble model accurately predicts the last time point for TMAF.

FIG. 21A-FIG. 21C depict non-limiting exemplary embodiments for using partial least squares (PLS) to guide possible modifications and recommendations.

FIG. 22 depicts the structure and some non-limiting exemplary uses for malonic acid.

FIG. 23 depicts an exemplary flow diagram of kinetic modeling.

FIG. 24 depicts non-limiting exemplary embodiments of 6 DBTL cycles.

FIG. 25 depicts an exemplary machine learning workflow with multi-omics data.

FIG. 26 depicts non-limiting exemplary embodiments related to dealing with data issues.

FIG. 27 depicts an exemplary flow diagram showing that machine learning, synthetic biology, and automation can complement each other perfectly, as illustrated by the methods disclosed herein.

FIG. 28 is a schematic illustration comparing methods of kinetic modeling based on ordinary differential equations and based on machine learning. The machine learning (ML) method uses time-series proteomics data to predict time-series metabolomics data (FIG. 2). The machine learning approach can complement, or supplement, a method based on ordinary differential equations where the change in metabolites over time is given by Michaelis-Menten kinetics (FIGS. 31 and 34). The machine learning method disclosed herein uses a time series of proteomics and metabolomics data to feed machine learning processes in order to predict pathway dynamics (Eq. (1) and FIG. 30). The machine learning method may require more data for training and/or make more accurate predictions. The method of the disclosure may be automatically applied to any pathway or host, thus leverages systematically new data sets to improve accuracy, and captures dynamic relationships which are unknown experimentally or have a different dynamic form Michaelis-Menten kinetics.

FIG. 29 shows a schematic illustration of a method for learning metabolic pathway dynamics from time series proteomics and metabolomics data. The method can be cyclic such that the metabolic system dynamics can be learned from time-series proteomics and metabolomics data, which can then be used to suggest new strain designs. At block 2904, experimentally, time-series proteomics and metabolomics data are acquired for several strains of interest (time-series proteomics and metabolomics data from three strains of interest are represented by the three lines.). These data are represented in a metabolomics phase space, with an axis corresponding to each measured metabolite. At block 2908, the time-series data traces are smoothed and differentiated (FIG. 32). The derivatives can be used as the training data to derive the relationship between metabolomics and proteomics data and the metabolite change (FIG. 30, Eq. (1)). At block 2912, the state derivative pairs are fed into a machine learning process, such as a supervised machine learning process. The machine learning process learns and generalizes the system dynamics from the examples provided by each strain. At block 2916, the model can then be used to simulate virtual strains and explore the metabolic space looking for mechanistic insight or valuable designs (such as commercially valuable designs). This process can then be repeated using the model to create new strains, which can further improve the accuracy of the dynamic model in the next round.

FIG. 30 shows a table of inputs and outputs to the machine learning model. In one embodiment, the core of the method consists in using one or more machine learning methods to predict the functional relationship between the metabolite derivative and proteomics and metabolomics data, substituting the traditional Michaelis-Menten relationship. The machine learning approach involved training a model for each metabolite that is being predicted (Table 1). Each model took all the measured metabolites and proteins at a particular time tᵢ as input. The prediction it provided as an output is the derivative of one of the pathway metabolites at the same time instant. The symbols \tilde{m} and \tilde{p} denote the experimentally measured metabolomic and proteomics measurements, respectively.

FIG. 31 shows a schematic illustration of limonene and isopentenol metabolic pathways. The machine learning method was tested on the limonene and isopentenol metabolic pathways. The limonene and isopentenol production pathways are variants of the mevalonate pathway. Time-series proteomics and metabolomics data were used to learn the dynamics of both the isopentenol and limonene producing strains. Additionally, a kinetic model was created and compared to the machine learning approach for the more complex limonene production pathway (FIG. 34). This pathway model was also used to generate simulated data to further evaluate the scaling properties of the proposed machine learning method.

FIG. 32 is a line plot showing computing derivative from metabolomics training data. A set of data points for a particular metabolite were used. In this case, \tilde{m}₃ had been measured at six time points. An interpolated and smoothed time series was created from the measurements, m̅₃(t), to reduce the noise of the signal and smooth the resulting derivative. The derivative of the time series was estimated by taking the derivative of the smoothed line at the time point of interest.

FIG. 33 is a plot showing cross validation and training scores as a function of training set size. This is a representative example of how model performance increased with the size of the data set provided. Cross validation techniques trained multiple models with a subset of the training data, and then test these model on data not used for training. In this case the training examples involved the time points for which derivatives were calculated from the training data, and proteomics and metabolomics data were available. In the training set each time series contained seven data points. These were too sparse to formulate accurate models. To overcome this a data augmentation scheme was employed where seven time points from the original data were expanded into 200 for each strain. This was done by filtering the data and interpolating over the filtered curve. In the plot, two data augmented strains were used where 360 points were used in the training set and 40 points were used in the test set.

FIG. 34 shows differential equations for a limonene pathway kinetic Michaelis-Menten model. This kinetic model was compiled from sources in the BRaunschweig ENzyme DAtabase (BRENDA) database. This model includes ten nonlinear ordinary differential equations, which describe the concentration for each metabolite in the pathway. The dynamics of this Michaelis-Menten model are complex enough to pose a significant challenge for machine learning techniques. This model was used to: (1) compare its predictions with machine learning predictions, and (2) generate simulated data sets to check scaling dependencies with the amount of time series used for training of machine learning processes. The machine learning method can be used to supplement, complement, or substitute these Michaelis-Menten expressions (see FIG. 30). Kinetic constants were left as free parameters when fitting experimental data shown in FIGS. 37A-37F.

FIG. 35 is a schematic illustration showing a set of reactions and inhibition relationships. The metabolites are shown inside rectangles, the enzymes are shown inside circles, solid arrows indicate forward flow into the next component, and dashed arrows indicate an inhibition relationship between the two species.

FIGS. 36A-36F show line graphs illustrating that the machine learning method produced good predictions of metabolite time series from proteomics data for the isopentenol producing E. coli strain. The measured metabolomics and proteomics data for the highest and lowest producing strains (training set data, red line) were used to train a model and learn the underlying dynamics (FIG. 29). The model was then tested by predicting the metabolite profiles (blue line) for a strain the model has never seen (medium producing strain, test data in green). A perfect prediction (blue line) would perfectly track the test data set (green line). Reasonable qualitative agreement was achieved even with only two time-series (strains) as training data. From a purely quantitative perspective, the average error could be improved: the total RMSE for the strain predictions was 40.34, which can be translated to 149.2% average error. However, for some metabolites the predictions quantitatively reproduced the measured data: Acetyl-CoA and isopentenol (the final product, which may be highly relevant for guiding bioengineering). For some metabolites (mevalonate, mevalonate phosphate and IPP/DMAPP), the model qualitatively reproduced the metabolite patterns, with scaling factors that may be improved. For HMG-CoA, the model can be further improved in the predictions of the metabolite concentration over time both quantitatively and qualitatively.

FIGS. 37A-37F show line graphs illustrating that the machine learning method outperformed the handcrafted kinetic model for the limonene producing E. coli strain. The only metabolite for which the kinetic model (black line) provided a better fit than the machine learning method (blue line) was mevalonate phosphate, although both methods appeared to track limonene (final product) production fairly well. The machine learning approach provided acceptable quantitative fits for Acetyl-CoA, HMG-CoA, and limonene, a qualitative description of metabolite behavior missing the scale factor for mevalonate, and did not provide either a qualitative description nor quantitatively accurate fit for mevalonate phosphate and IPP/DMAPP. As in FIGS. 36A-36F, the experimentally measured profiles corresponded to high, low and medium producers of limonene. The training sets were the low and high producers (in red), and the model was used to predict the concentrations for the medium producing strain (in green). Kinetic constants for the handcrafted kinetic model in FIG. 34 were left as free parameters when fitting the experimental data.

FIG. 38 is a bar chart showing that prediction errors decreased markedly with increasing training set size. As the number of available proteomics and metabolomics times-series data sets (strains) for training increased, the prediction error (RMSE, Eq. (6)) decreased conspicuously. Moreover, the standard deviation of the predictions error (vertical bar) decreased notably as well. The change from 2 to 10 strains was more pronounced that the change from 10 to 100. This observation indicated that it would be more productive to do ten rounds of metabolic engineering collecting ten time-series data sets, than a single round collecting 100 time series.

FIGS. 39A-39J show line graphs illustrating that predictions improved with more training data sets. The machine learning process was used to predict kinetic models for varying sizes of training sets (2, 10, and 100 virtual strains in blue, red and black). Ten unique training sets were used for each size to show prediction variability (shown by the shadings) for each training set size. All models converge towards the actual dynamics with the 100 strain models in closest agreement. Standard deviations (shown by the shadings) also decreased markedly as the size of the training set increases.

FIGS. 40A and 40B show how the success rate of predicting production ranks increased with training set size. FIG. 40A is a bar chart showing the success rate in predicting the relative production order (i.e., which strain produced most, which one produced least and which one was a medium producer) for groups of three time series (strains) randomly chosen from a pool of 10,000 strains, as a function of training data set size (strains). For 100 data sets, the failure rate to predict the top producer was <10%. For ten data sets the success rate was ˜80%, which was reliable enough to guide engineering efforts. The horizontal line provided the rate of success (1/6) if order is chosen randomly. FIG. 40B is a plot showing that prediction of limonene production was extremely accurate for the case of a training data set comprised of 100 time-series (strains). These data shows that the machine learning model predictions were accurate enough to guide pathway design if enough training data is available.

FIG. 41A is a plot and FIG. 41B is a line graph that show that a machine learning (ML) approach can be used to produce biological insights. FIG. 41A shows the final position in the proteomics phase space (similarly to the PCAP approach) for 50 strains generated by the ML process by learning from the Michaelis-Menten kinetic model (FIG. 34) used as ground truth. Final limonene production is given by circle size and color. The PLS process found directions in the proteomics phase space that best align with increasing limonene production (component 1). Traveling in proteomics phase space along that direction (which involved overexpression of LS and underexpression of AtoB, PMD, and Idi, see Table 2) created strains with higher limonene production. The ML approach not only produced biological insights to increase production but also predicted the expected concentration as a function of time for limonene and all other metabolites, generating hypotheses that can be experimentally tested (right panel).

FIG. 42 is a block diagram of an illustrative computing system that can be used in some embodiments to execute the processes and implement the features described herein.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

New synthetic biology capabilities hold the promise of dramatically improving our ability to engineer biological systems. However, a fundamental hurdle in realizing this potential is the inability to accurately predict biological behavior after modifying the corresponding genotype. Kinetic models have traditionally been used to predict pathway dynamics in bioengineered systems, but they take significant time to develop, and rely heavily on domain expertise. The methods of the present disclosure can effectively predict pathway dynamics in an automated fashion using a combination of machine learning and abundant multiomics data (proteomics and metabolomics). The methods outperform a classical kinetic model, and produces qualitative and quantitative predictions that can be used to productively guide bioengineering efforts. This method systematically leverages arbitrary amounts of new data to improve predictions, and does not assume any particular interactions, but rather implicitly chooses the most predictive ones.

### Kinetic Learning

Disclosed herein include methods for simulating a virtual strain of an organism. In some embodiments, a method for simulating a virtual strain of an organism comprises receiving time-series multiomics data of an organism, wherein the times-series multiomics data comprises time-series proteomics data of a plurality of proteins and time-series metabolomics data comprising a characteristic of a metabolite. The method can comprise training a machine learning model with time-series proteomics data as input and the time-series metabolomics data of the metabolite as output. The method can comprise simulating a virtual strain of the organism using the machine learning model to determine the characteristic of the metabolite in the virtual strain.

In some embodiments, the time-series multiomics data comprises time-series multiomics data of a plurality of strains of the organism. In some embodiments, the time-series proteomics data is associated with a metabolic pathway. In some embodiments, wherein the metabolic pathway comprises a heterologous pathway. In some embodiments, the machine learning model represents kinetics of the metabolic pathway.

In some embodiments, the characteristic of the metabolite is a titer, rate, concentration, or yield of the metabolite. In some embodiments, the proteomics data comprises a concentration of each of a plurality of proteins at each of a plurality of time points, and wherein the metabolomics data comprises a concentration of the metabolite at each of the plurality of time points. In some embodiments, the multiomics data comprises replicates (e.g., duplicates, triplicates, quadruplicates, quintuplicates, sextuplicates, septuplicates, octuplicates, or more) of a concentration of a protein at a time point. The multiomics data can comprise replicaties (e.g., duplicates, triplicates, quadruplicates, quintuplicates, sextuplicates, septuplicates, octuplicates, or more) of a concentration of the metabolite at a time point. In some embodiments, simulating the virtual strain of the organism comprises determining a concentration of the metabolite of the virtual strain using the machine learning model.

The times-series multiomics data can comprise, for example, multiomics data, genomics data, proteomics data, transcriptomics data, epigenomics data, metabolomics data, chromatics data, cytokine secretion data, or a combination thereof. The times-series multiomics data can comprise different types of data, such as proteomics, metabolomics, HPLC, bioreactor, OD600, or a combination thereof. Each type of data can comprise multiple measurements, such as 1, 2, 3, 4, 5, 6, 7, 8, 9, 10, 20, 30, 40, 50, 60, 70, 80, 90, 100, 150, 200, 300, 400, 500, 600, 700, 800, 900, 1000, or a number or a range between any two of these values. For example, proteomics data can include data (such as concentrations) of 63 proteins. The metabolomics data can include data (such as concentrations) of a number of meteorites, such as 72 metabolites. The HPLC data can include HPLC data of 11 metabolites. The bioreactor data can include, for example, 6 measurements, such as Total Malonic Acid Formed (TMAF), pH, DCW, DO, CO2, O2. The multiomics data can include OD600 readings.

Exemplary proteins can comprise: (R,R)-butanediol dehydrogenase; 1,3-beta-glucanosyltransferase; 3-hydroxyisobutyryl-CoA hydrolase; 6-phosphogluconate dehydrogenase, decarboxylating; ATP-dependent 6-phosphofructokinase; Acetyl-CoA acetyltransferase IA; Acetyl-CoA carboxylase; Acetyl-CoA hydrolase; Acetyl-coenzyme A synthetase; Aconitate hydratase, mitochondrial; Adenylate kinase; Alcohol dehydrogenase 3; Alcohol dehydrogenase 4, mitochondrial; Aldehyde dehydrogenase; Aldehyde dehydrogenase 5, mitochondrial; Alpha,alpha-trehalose-phosphate synthase [UDP-forming]; Citrate synthase; Dihydrolipoyl dehydrogenase; Dihydrolipoyllysine-residue succinyltransferase component of 2-oxoglutarate dehydrogenase complex, mitochondrial; Enolase 1; External NADH-ubiquinone oxidoreductase 1, mitochondrial; Fatty acid synthase subunit alpha; Fatty acid synthase subunit beta; Fructose-bisphosphate aldolase; Glucose-6-phosphate isomerase; Glyceraldehyde-3-phosphate dehydrogenase; Glycogen [starch] synthase; Inorganic pyrophosphatase; Isocitrate dehydrogenase [NADP]; Isocitrate dehydrogenase [NAD] subunit 1, mitochondrial; Isocitrate dehydrogenase [NAD] subunit, mitochondrial; Isocitrate lyase; Malate dehydrogenase; NAD-dependent malic enzyme, mitochondrial; NADH dehydrogenase (Quinone), G subunit; NADH dehydrogenase [ubiquinone] flavoprotein 1, mitochondrial; NADH dehydrogenase [ubiquinone] iron-sulfur protein 7, mitochondrial; NADH-ubiquinone oxidoreductase 24 kDa subunit, mitochondrial; NADH-ubiquinone oxidoreductase 49 kDa subunit, mitochondrial; NADP-dependent alcohol dehydrogenase 6; Phosphoenolpyruvate carboxykinase [ATP]; Phosphoglycerate kinase; Phosphotransferase; Potassium-activated aldehyde dehydrogenase, mitochondrial; Pyruvate carboxylase; Pyruvate decarboxylase isozyme 3; Pyruvate dehydrogenase E1 component subunit beta; Pyruvate kinase; Succinate dehydrogenase [ubiquinone] cytochrome b small subunit; Succinate dehydrogenase [ubiquinone] flavoprotein subunit, mitochondrial; Succinate dehydrogenase [ubiquinone] iron-sulfur subunit, mitochondrial; Succinate-CoA ligase [ADP-forming] subunit beta, mitochondrial; Transaldolase; Transketolase; Triosephosphate isomerase; UTP-glucose-1-phosphate uridylyltransferase; and/or YPL061Wp-like protein.

The metabolites can be intracellular as well as extracellular metabolites. The intracellular metabolites can include, for example, oxalacetic acid, oxalate, NADP+, succinyl-CoA, malonate, L-tyrosine, L-glutamic acid, Methylmalonic acid, coenzyme A, trehalose, Cytidine triphosphate, cis-Aconitic acid, L-methionine, fumarate, lactic acid, Sedoheptulose 7-phosphate, Glutathione oxidized form, isopentenyl pyrophosphate, (R)-mevalonate, thymidylic acid, acetyl-CoA, uridine 5′-triphosphate, 5′-Guanylic acid, L-threonine, Uridine 5′-monophosphate, D-Glucose, Fructose 6-Phosphate, pyruvate, DL-Glyceraldehyde 3-phosphate, trehalose-6-phosphate, glyoxylate, malic acid, ribose-5-phosphate, Methylmalonyl coa, succinate, NADPH, L-leucine, 3-phosphoglycerate, acetylphosphate, cis-4-coumarate, stearoyl-CoA, phosphoenolpyruvate, beta-D-Fructose 1,6-bisphosphate, L-aspartic acid, Guanosine 5′-diphosphate, L-histidine, adenosine 5′-monophosphate, palmitoyl-CoA, 2-ketoglutaric acid, malonyl-CoA, dihydroxyacetone phosphate, Cytidine 5′-diphosphate, L-arginine, flavin adenine dinucleotide, NADH, biotin, D-Glucose 6-phosphate, Uridine 5′-diphosphate, deoxy-TDP, 6-phosphogluconic acid, 5′-cytidylic acid, guanosine triphosphate, D-Arabinitol, Adenosine 5′-diphosphate, D-Erythrose 4-phosphate, propionyl-CoA, dTTP, L-phenylalanine, Adenosine triphosphate, L-serine, Glutathione, and/or nadide. The metabolites measured can involve intracellular as well as extracellular metabolites. The extracellular metabolites can include, for example, pyruvate, malonate, ethanol, citrate, trehalose, acetate, D-Arabinitol, glycerol, uracil, succinate, and/or D-Glucose.

The times-series multiomics data can include times-series multiomics data of a number of strains and/or a number of replicates. The times-series multiomics data can include times-series multiomics data of multiple strains, such as 2, 3, 4, 5, 6, 7, 8, 9, 10, 15, 20, 24, 25, 30, 35, 40, 50, 60, 70, 80, 90, 100, or a number or a range between any two of these values. The times-series multiomics data can include replicates, such as 2, 3, 4, 5, 6, 7, 8, 9, 10, 15, 20, 30, 40, 50, or a number or a range between any two of these values, replicates. The times-series can include a number of time points, such as 2, 3, 4, 5, 6, 7, 8, 9, 10, 20, 30, 40, 50, 60, 70, 80, 90, 100, or a number or a range between any two of these values, time points.

In some embodiments, the time-series multiomics data comprises first time-series multiomics data and second time-series multiomics data. The first time-series multiomics data can comprise time-series metabolomics data of a plurality of strains of an organism, wherein the time-series metabolomics data comprises two or more time-series of a strain. The second time-series multiomics data can comprise time-series proteomics data of a plurality of strains of an organism, and wherein the time-series proteomics data comprises a plurality of time-series of a strain. The first time-series multiomics data can comprise time-series multiomics data of a plurality of strains of an organism, and wherein the first time-series multiomics data comprises time-series multiomics data of a plurality of strains of a different organism.

The first time-series multiomics data or the second time-series multiomics data comprises time-series proteomics data, time-series metabolomics data, time-series transcriptomics data, or a combination thereof. The first time-series multiomics data or the second time-series multiomics data can be associated with an enzymatic characteristic selected from the group consisting of a k_{cat} constant, a Kₘ constant, and a kinetic characteristics curve. The first time-series multiomics data and the second time-series multiomics data can comprise observations at corresponding time points.

In some embodiments, the machine learning model comprises a supervised machine learning model. In some embodiments, the machine learning model comprises a non-classification model, a neural network, a recurrent neural network (RNN), a linear regression model, a logistic regression model, a decision tree, a support vector machine, a Naïve Bayes network, a k-nearest neighbors (KNN) model, a k-means model, a random forest model, a multilayer perceptron, or a combination thereof. In some embodiments, the machine learning model comprises a deep neural network (DNN), deep recurrent neural network (DRNN), gated recurrent unit (GRU) DRNN, a partial least square (PLS) model, or a combination thereof. In some embodiments, the machine learning model comprises an ensemble model of a plurality of machine learning models (e.g., 2, 3, 4, 5, 6, 7, 8, 9, 10, 20, 30, 40, 50, or more, machine learning models). The plurality of machine learning models can comprise a deep neural network (DNN), deep recurrent neural network (DRNN), and gated recurrent unit (GRU) DRNN.

In some embodiments, the virtual strain comprises an increased expression of at least one first protein, a knock-out of at least one second protein, a reduced expression of at least one third protein, or a combination thereof. In some embodiments, the at least one first protein comprises at least 2, 3, 4, 5, 6, 7, 8, 9, 10, 20, 30, 40, 50, 60, 70, 80, 90, 100, or more, first proteins. In some embodiments, the at least one second protein comprises at least 2, 3, 4, 5, 6, 7, 8, 9, 10, 20, 30, 40, 50, 60, 70, 80, 90, 100, or more, second proteins. In some embodiments, the at least one third protein comprises at least 2, 3, 4, 5, 6, 7, 8, 9, 10, 20, 30, 40, 50, 60, 70, 80, 90, 100, or more, third proteins.

In some embodiments, the method comprises designing one or more new strains based on the virtual strain. The method can comprise receiving experimental time-series multiomics data for the new strains. The method can comprise retraining the machine learning model based on the experimental time-series multiomics data of the new strains.

A time series data can comprise a number of time points, such as 4, 5, 6, 7, 8, 9, 10, 20, 30, 40, 50, 60, 70, 80, 90, 100, or more, time points. In some embodiments, the method comprise interpolating the time-series multiomics data (or a subset of the time-series multiomics data) from a first number of time points to a second number of time points. In some embodiments, the first number of time points comprises, 4, 5, 6, 7, 8, 9, 10, 20, 30, 40, 50, 60, 70, 80, 90, 100, or more, time points. In some embodiments, the second number of time points comprises 50, 55, 56, 57, 58, 59, 60, 61, 62, 63, 64, 65, 70, 75, 80, 90, 100, or more, time points. The first number of time points can be time points every hour, 2 hours, 3 hours, 4 hours, 5 hours, 6 hours, or more. The second number of time points can be hourly time points. The second number of time points can be time points every 30 minutes, 1 hour, 2 hours, 3 hours, 4 hours, 5 hours, 6 hours, or more. Interpolating the time-series multiomics can data comprise interpolating the time-series multiomics data using a cubic spline method.

Disclosed herein include methods of stimulating a strain of an organism. In some embodiments, a method of stimulating a strain of an organism comprises receiving time-series multiomics data of a plurality of strains of an organism comprising time-series proteomics data of a plurality of proteins and time-series metabolomics data comprising a characteristic of a metabolite. The method can comprise training a machine learning model with the time-series proteomics data as input and the time-series metabolomics data of the metabolite as output. The method can comprise simulating a virtual strain of the organism using the machine learning model to determine the characteristic of the metabolite in the virtual strain.

In some embodiments, receiving the time-series multiomics data comprises data checking and/or preprocessing of the time-series multiomics data of the plurality of strains of the organism.

In some embodiments, the time-series multiomics data comprises multiomics data of two or more time-series of a strain, such as 3, 4, 5, 6, 7, 8, 9, 10, 20, 30, 40, 50, 100, or more. In some embodiments, the time-series multiomics data comprises time-series proteomics data, time-series metabolomics data, time-series transcriptomics data, or a combination thereof. In some embodiments, the multiomics data comprises observations of each of a plurality of proteins at a plurality of time points and observations of the metabolite at the plurality of time points.

In some embodiments, the machine learning model comprises a supervised machine learning model. In some embodiments, machine learning model comprises a deep neural network (DNN), deep recurrent neural network (DRNN), gated recurrent unit (GRU) DRNN, a partial least square (PLS) model, or a combination thereof. In some embodiments, the machine learning model comprises an ensemble model of a plurality of machine learning models, optionally wherein the plurality of machine learning models comprises a deep neural network (DNN), deep recurrent neural network (DRNN), and gated recurrent unit (GRU) DRNN.

In some embodiments, simulating the virtual strain of the organism comprises simulating the virtual strain of the organism using the machine learning model to change one or more of titer, rate, concentration, and yield of the metabolite.

In some embodiments, the method comprises comprising designing a strain of the organism corresponding to the virtual strain. In some embodiments, the method comprises creating a strain of the organism corresponding to the virtual strain.

Disclosed herein include methods for determining modifications of protein expression an organism. In some embodiments, a method for determining modifications of protein expression of an organism comprises: receiving time-series multiomics data of a plurality of strains of an organism comprising time-series proteomics data of comprising a characteristic of each of a plurality of proteins and time-series metabolomics data comprising a characteristic of a metabolite. The method can comprise training a machine learning model with the time-series proteomics data as input and the time-series metabolomics data of the metabolite as output. The method can comprise determining modifications of a concentration of each of one or more proteins using the machine learning model.

In some embodiments, the characteristic of each of the plurality of proteins comprises a concentration of the protein, and/or wherein the characteristic of the metabolite comprises a concentration of the metabolite. In some embodiments, the modifications comprise an increased expression of at least one first protein, a knock-out of at least one second protein, a reduced expression of at least one third protein, or a combination thereof, optionally wherein the at least one first protein comprises at least 10 first proteins, optionally wherein the at least one second protein comprises at least 10 second proteins, optionally wherein the at least one third protein comprises at least 10 third proteins.

### Guiding Metabolic Engineering Via Kinetic Deep Learning and Multi-Omics

Provided herein are methods of kinetic learning. Such methods can be purely data driven. A very large data set, for example of 480,000 data points, can be used to train a model or models, such as neural networks. Neural networks can be used to generate accurate predictions.

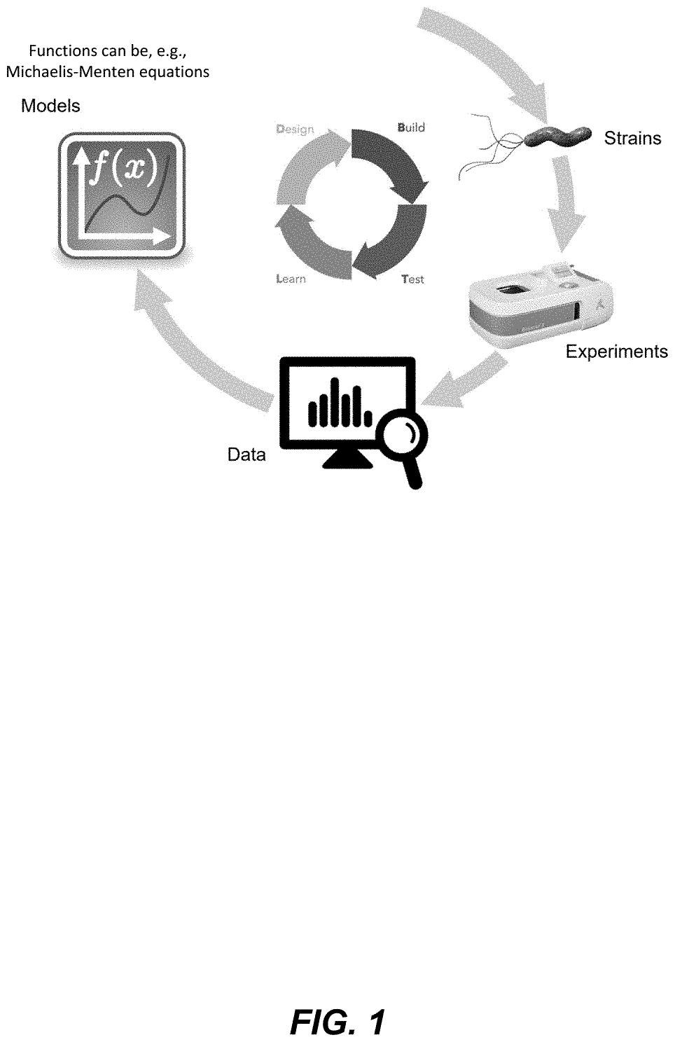

Kinetic modeling predicts metabolic behavior to produce a desired outcome (FIG. 1). When predictions fail, kinetic modeling (differential equations-based kinetic modeling) becomes complicated to systematically change the equations.

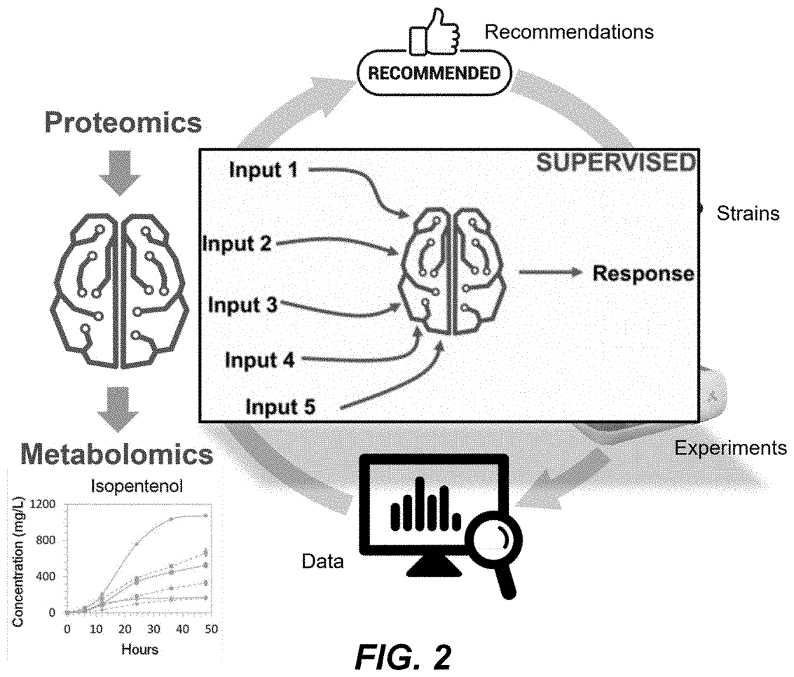

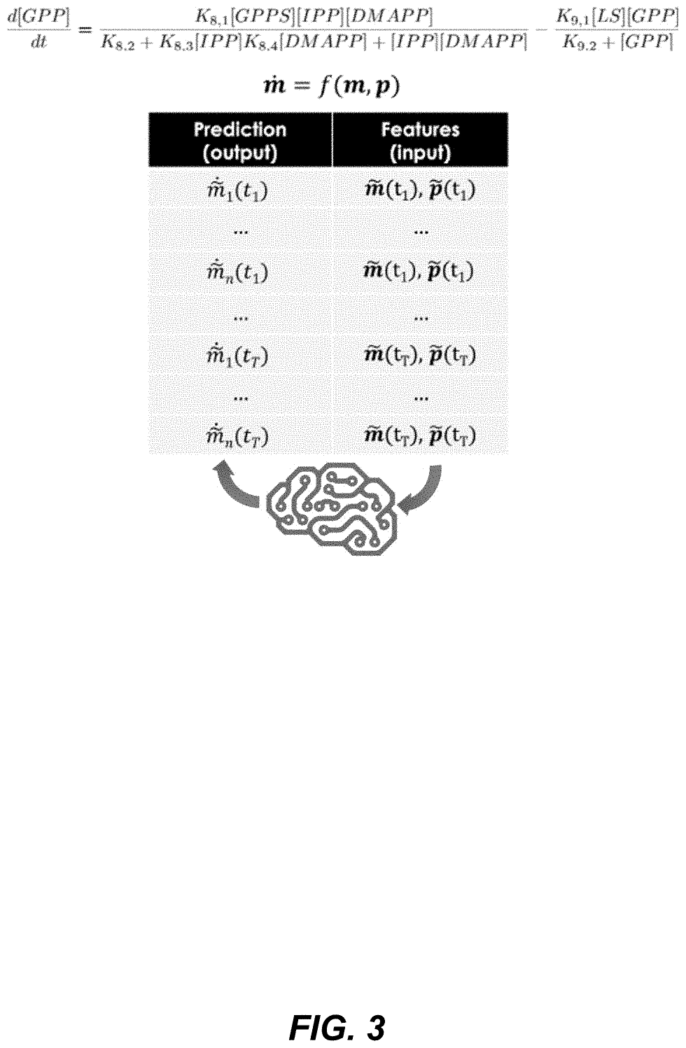

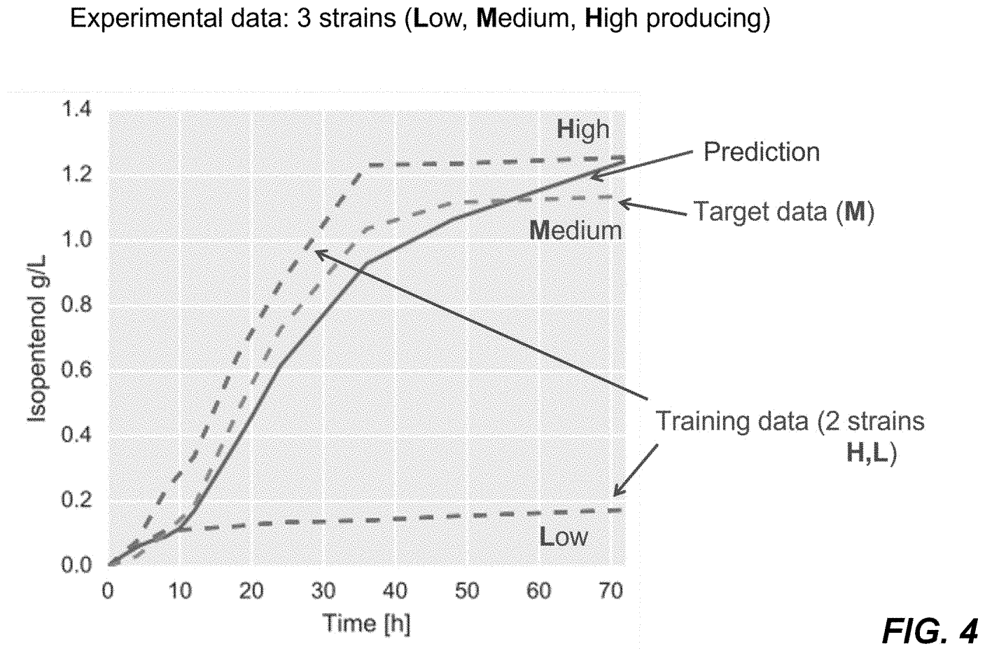

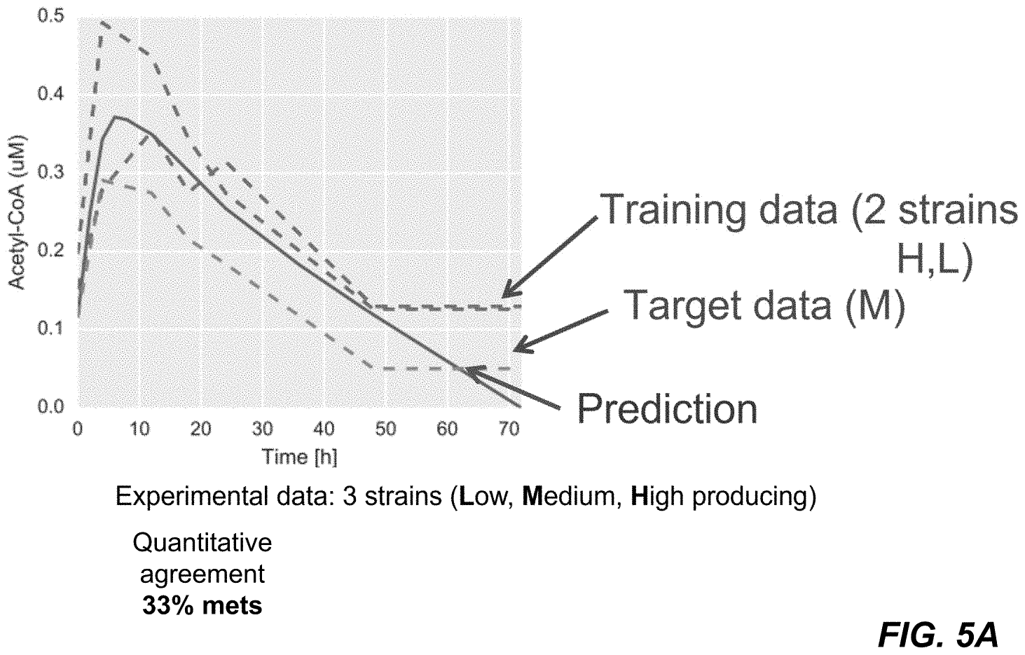

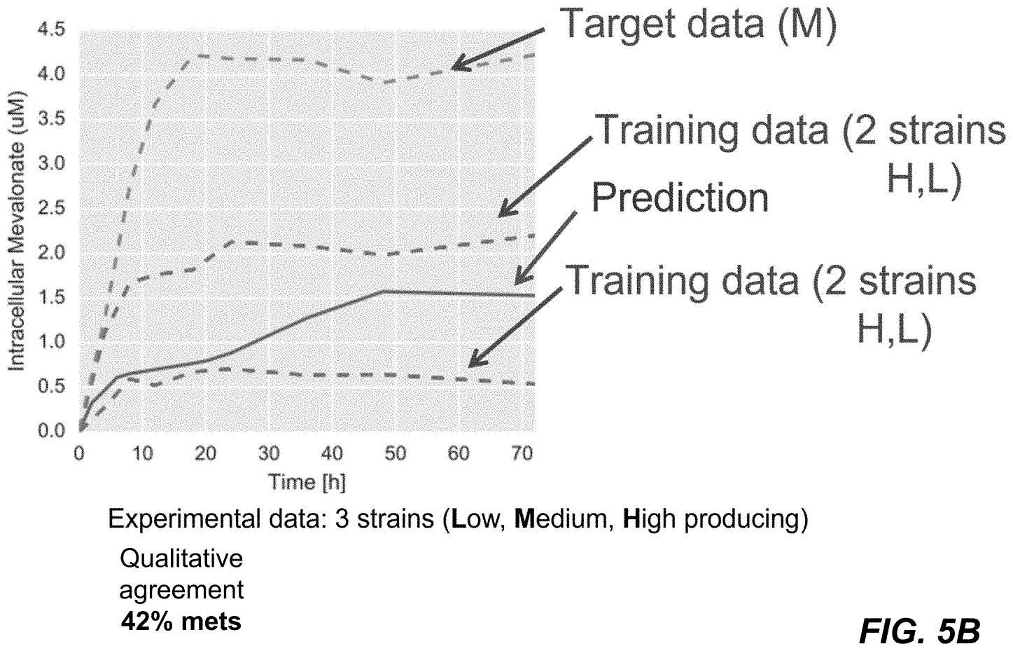

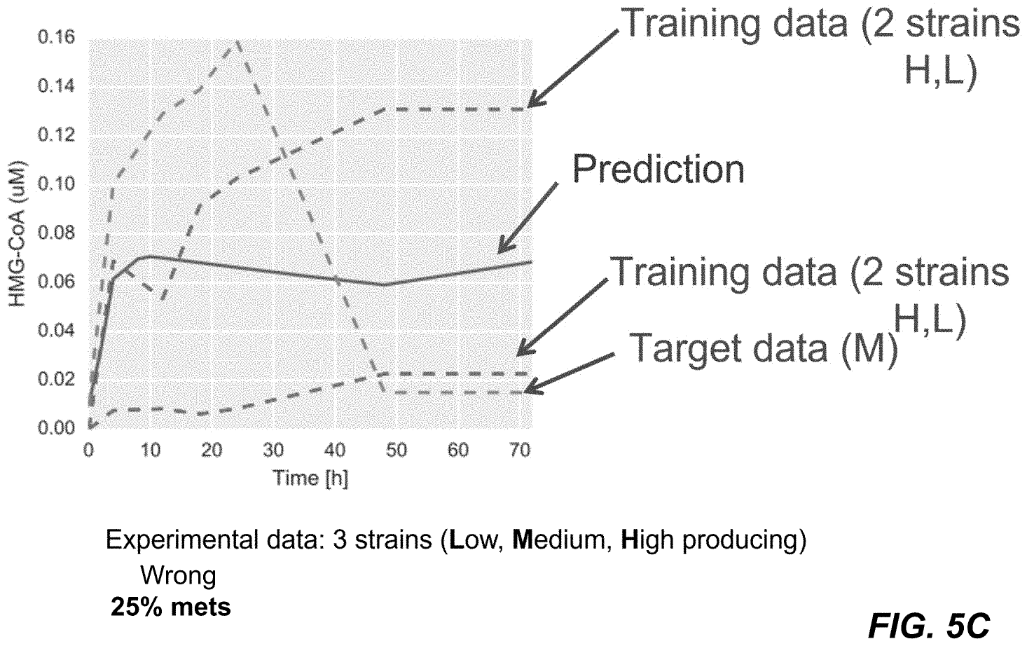

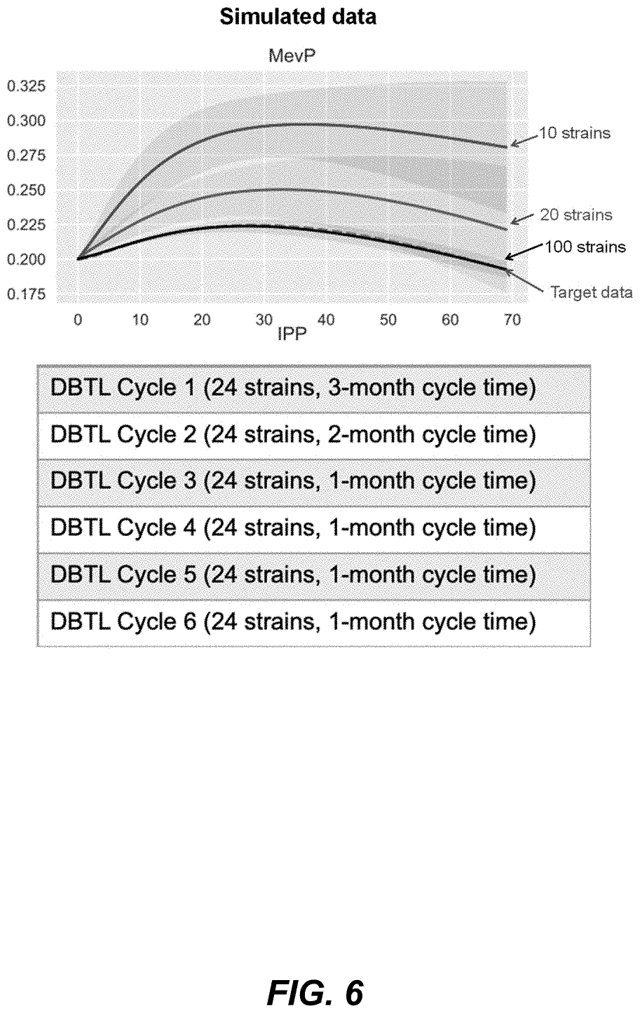

The methods disclosed herein can utilize machine learning to learn and predict kinetics. Performance improves as more data is added (FIG. 2). Machine learning can be used to relearn, e.g., Michaelis-Menten kinetic, more accurately (FIG. 3). The method can be derivative based. As shown in FIG. 4 and FIG. 5A-FIG. 5C, the predictions are accurate on final product concentrations and are mixed for other metabolites in the pathway As shown in FIG. 6, the predictions improve substantially as data for more strains are added. Pairwise feature interactions and features can be either design features like promoter strength or expression levels measured by proteomics.

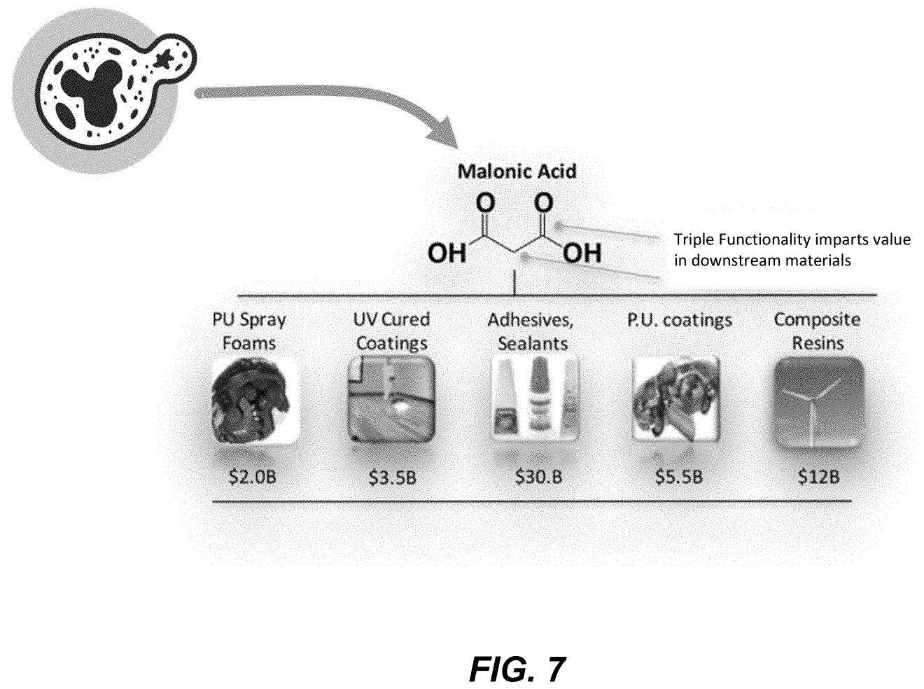

In an exemplary use of the machine learning methods provided herein, a goal is to improve production of malonic acid, an intermediate used for sundry final products, with over 150-years of use in synthetic chemistry. Malonic acid is difficult to produce from petrochemistry (<75% yields), and production is largely driven by foreign suppliers (FIG. 7).

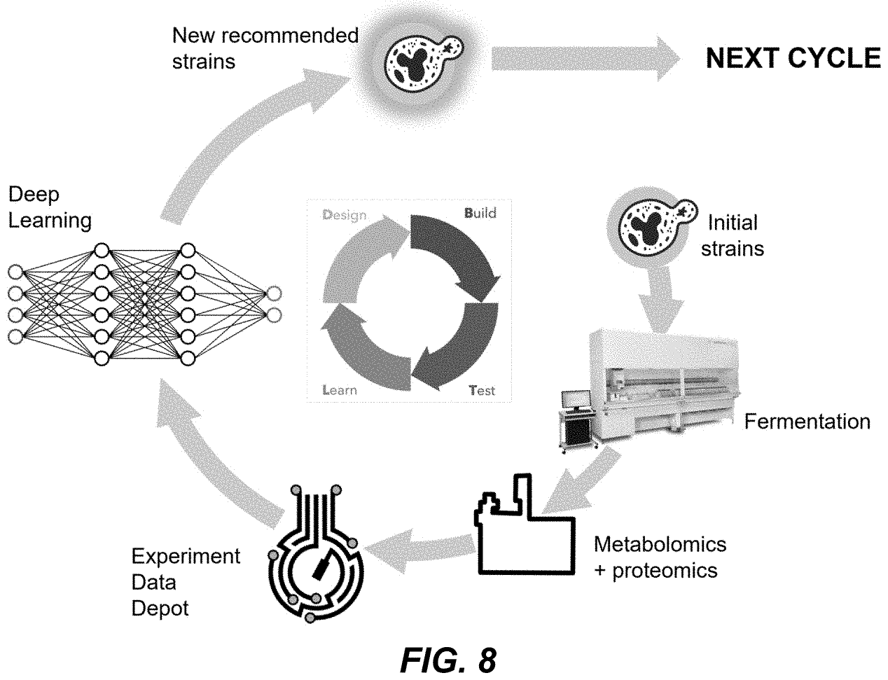

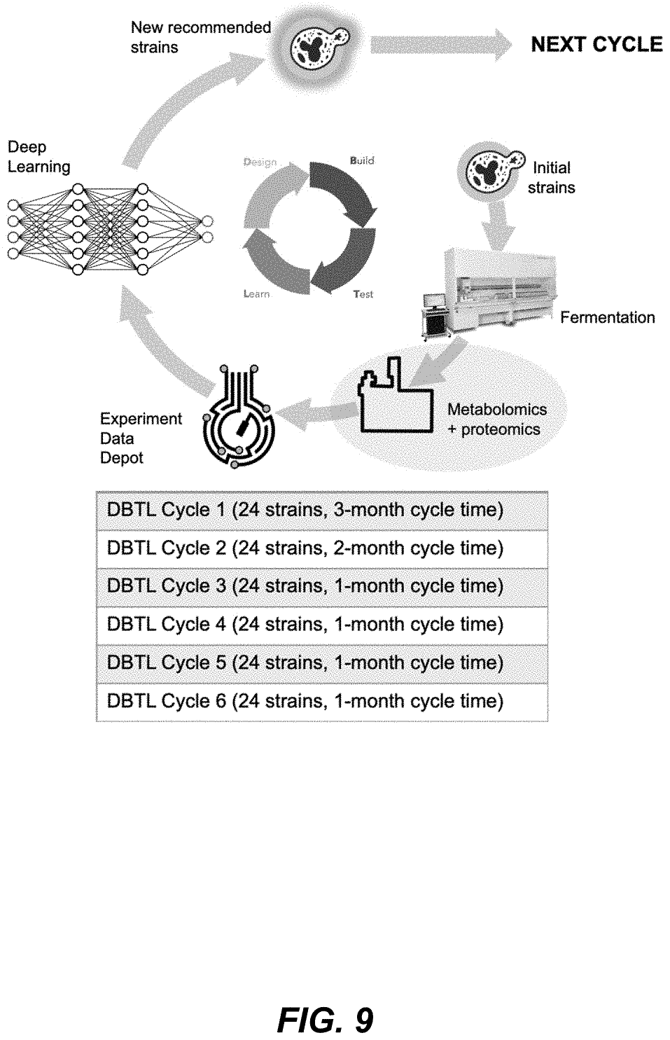

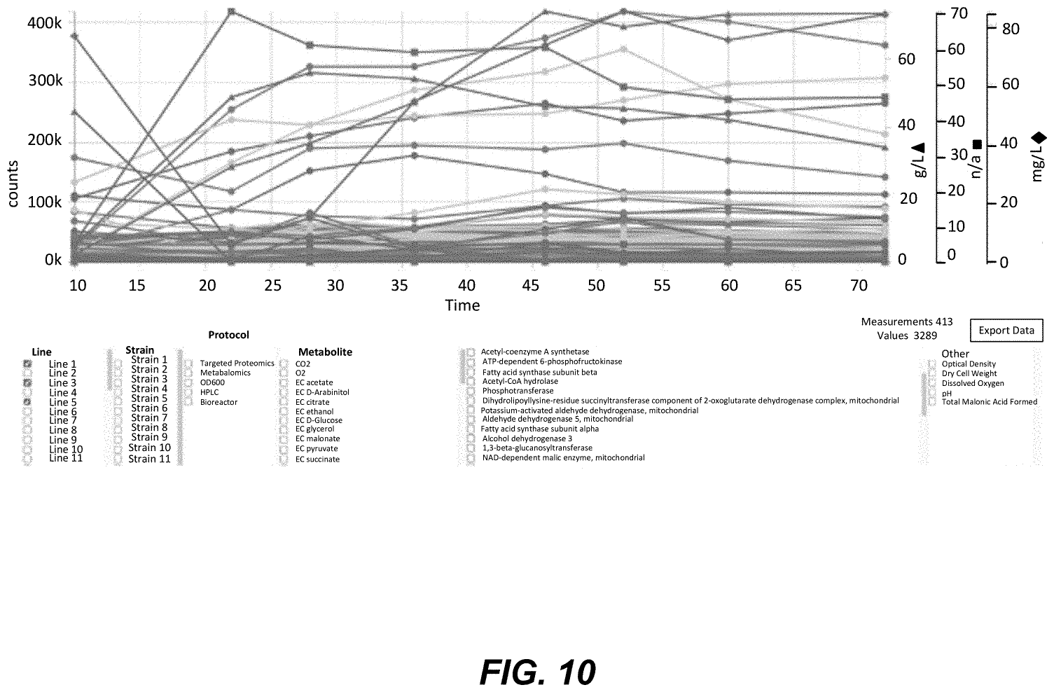

As shown in FIG. 8, workflows are interconnected and efforts are shared, with each cycle producing a new set of 24 strains to be improved in the next cycles (for each cycle of Design, Build, Test, Learn (DBTL)). In some embodiments, 6 DBTL cycles in total (FIG. 9) can be performed to gather the largest public multiomics data set as compared to previous datasets. Multi-omics time-series of 80,000 data points per DBTL cycle can be produced. The multi-omics data set can include: Proteomics (63 proteins), Metabolomics (72 metabolites), HPLC (11 metabolites), Bioreactor (6 measurements—Total Malonic Acid Formed (TMAF), pH, dry cell weight (DCW), dissolved oxygen (DO), CO2, O2), D600, 24 strains, 3 replicates, and 8-point time-series. Therefore, 24 strains×3 triplicates×8 time points×(60 proteins+100 metabolites)≅80,000 data points (FIG. 10).

For example, 63 proteins can be measured and involve central carbon metabolism as well as pathway proteins. The proteins can comprise: (R,R)-butanediol dehydrogenase; 1,3-beta-glucanosyltransferase; 3-hydroxyisobutyryl-CoA hydrolase; 6-phosphogluconate dehydrogenase, decarboxylating; ATP-dependent 6-phosphofructokinase; Acetyl-CoA acetyltransferase IA; Acetyl-CoA carboxylase; Acetyl-CoA hydrolase; Acetyl-coenzyme A synthetase; Aconitate hydratase, mitochondrial; Adenylate kinase; Alcohol dehydrogenase 3; Alcohol dehydrogenase 4, mitochondrial; Aldehyde dehydrogenase; Aldehyde dehydrogenase 5, mitochondrial; Alpha,alpha-trehalose-phosphate synthase [UDP-forming]; Citrate synthase; Dihydrolipoyl dehydrogenase; Dihydrolipoyllysine-residue succinyltransferase component of 2-oxoglutarate dehydrogenase complex, mitochondrial; Enolase 1; External NADH-ubiquinone oxidoreductase 1, mitochondrial; Fatty acid synthase subunit alpha; Fatty acid synthase subunit beta; Fructose-bisphosphate aldolase; Glucose-6-phosphate isomerase; Glyceraldehyde-3-phosphate dehydrogenase; Glycogen [starch] synthase; Inorganic pyrophosphatase; Isocitrate dehydrogenase [NADP]; Isocitrate dehydrogenase [NAD] subunit 1, mitochondrial; Isocitrate dehydrogenase [NAD] subunit, mitochondrial; Isocitrate lyase; Malate dehydrogenase; NAD-dependent malic enzyme, mitochondrial; NADH dehydrogenase (Quinone), G subunit; NADH dehydrogenase [ubiquinone] flavoprotein 1, mitochondrial; NADH dehydrogenase [ubiquinone] iron-sulfur protein 7, mitochondrial; NADH-ubiquinone oxidoreductase 24 kDa subunit, mitochondrial; NADH-ubiquinone oxidoreductase 49 kDa subunit, mitochondrial; NADP-dependent alcohol dehydrogenase 6; Phosphoenolpyruvate carboxykinase [ATP]; Phosphoglycerate kinase; Phosphotransferase; Potassium-activated aldehyde dehydrogenase, mitochondrial; Pyruvate carboxylase; Pyruvate decarboxylase isozyme 3; Pyruvate dehydrogenase E1 component subunit beta; Pyruvate kinase; Succinate dehydrogenase [ubiquinone] cytochrome b small subunit; Succinate dehydrogenase [ubiquinone] flavoprotein subunit, mitochondrial; Succinate dehydrogenase [ubiquinone] iron-sulfur subunit, mitochondrial; Succinate-CoA ligase [ADP-forming] subunit beta, mitochondrial; Transaldolase; Transketolase; Triosephosphate isomerase; UTP-glucose-1-phosphate uridylyltransferase; and/or YPL061Wp-like protein.

For example, 72 metabolites can be measured and involve intracellular as well as extracellular metabolites. The intracellular metabolites can include, but are not limited to: oxalacetic acid, oxalate, NADP+, succinyl-CoA, malonate, L-tyrosine, L-glutamic acid, Methylmalonic acid, coenzyme A, trehalose, Cytidine triphosphate, cis-Aconitic acid, L-methionine, fumarate, lactic acid, Sedoheptulose 7-phosphate, Glutathione oxidized form, isopentenyl pyrophosphate, (R)-mevalonate, thymidylic acid, acetyl-CoA, uridine 5′-triphosphate, 5′-Guanylic acid, L-threonine, Uridine 5′-monophosphate, D-Glucose, Fructose 6-Phosphate, pyruvate, DL-Glyceraldehyde 3-phosphate, trehalose-6-phosphate, glyoxylate, malic acid, ribose-5-phosphate, Methylmalonyl coa, succinate, NADPH, L-leucine, 3-phosphoglycerate, acetylphosphate, cis-4-coumarate, stearoyl-CoA, phosphoenolpyruvate, beta-D-Fructose 1,6-bisphosphate, L-aspartic acid, Guanosine 5′-diphosphate, L-histidine, adenosine 5′-monophosphate, palmitoyl-CoA, 2-ketoglutaric acid, malonyl-CoA, dihydroxyacetone phosphate, Cytidine 5′-diphosphate, L-arginine, flavin adenine dinucleotide, NADH, biotin, D-Glucose 6-phosphate, Uridine 5′-diphosphate, deoxy-TDP, 6-phosphogluconic acid, 5′-cytidylic acid, guanosine triphosphate, D-Arabinitol, Adenosine 5′-diphosphate, D-Erythrose 4-phosphate, propionyl-CoA, dTTP, L-phenylalanine, Adenosine triphosphate, L-serine, Glutathione, and/or nadide. The metabolites measured can involve intracellular as well as extracellular metabolites. The extracellular metabolites can comprise: pyruvate, malonate, ethanol, citrate, trehalose, acetate, D-Arabinitol, glycerol, uracil, succinate, and/or D-Glucose.

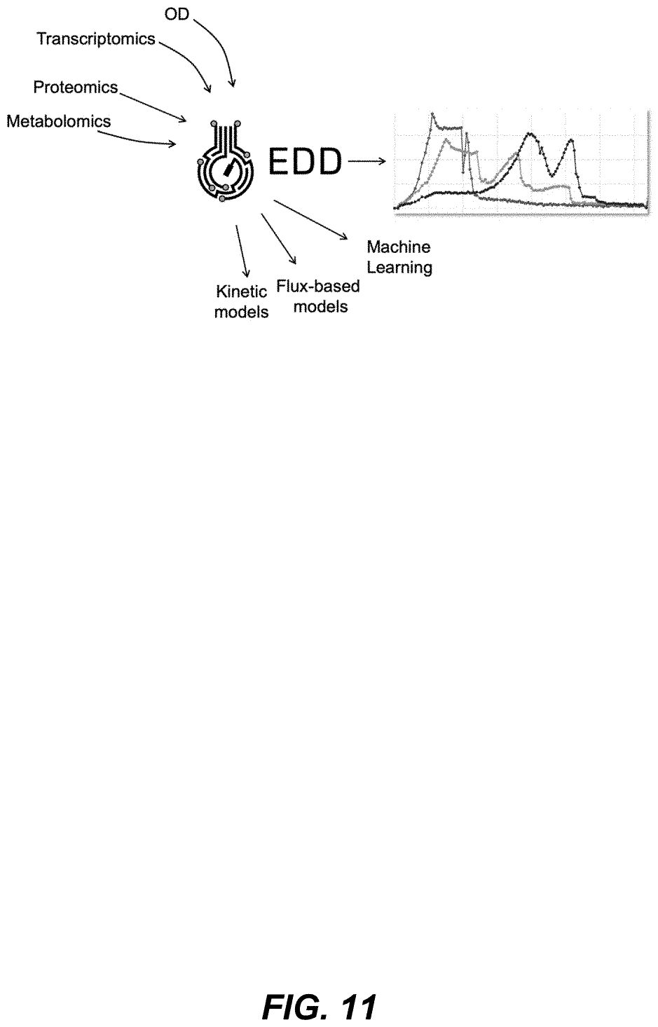

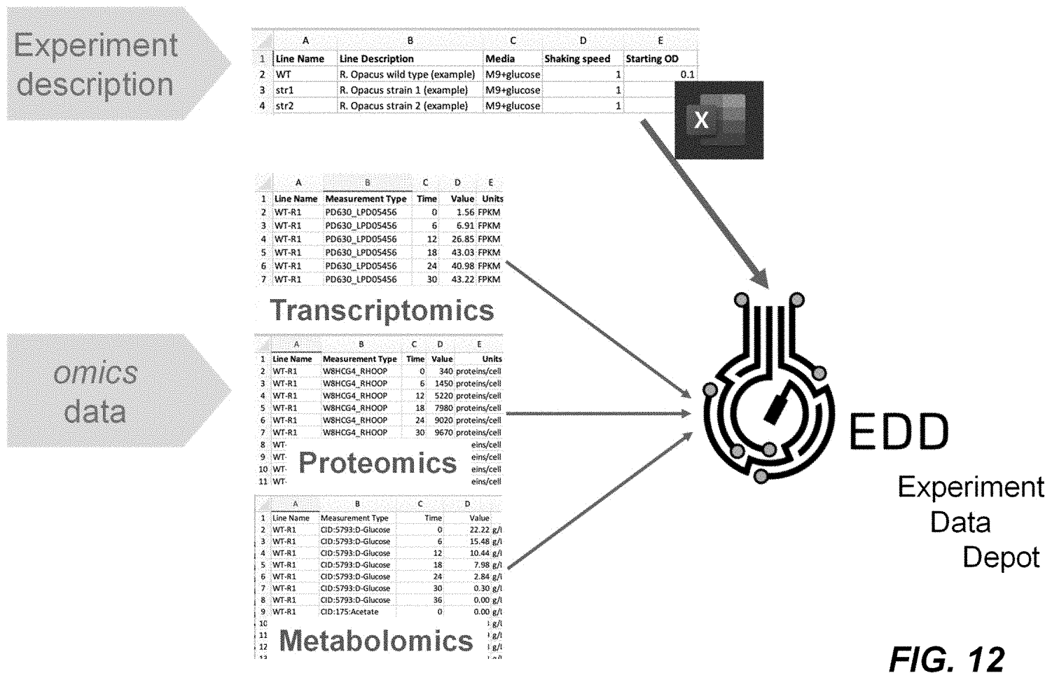

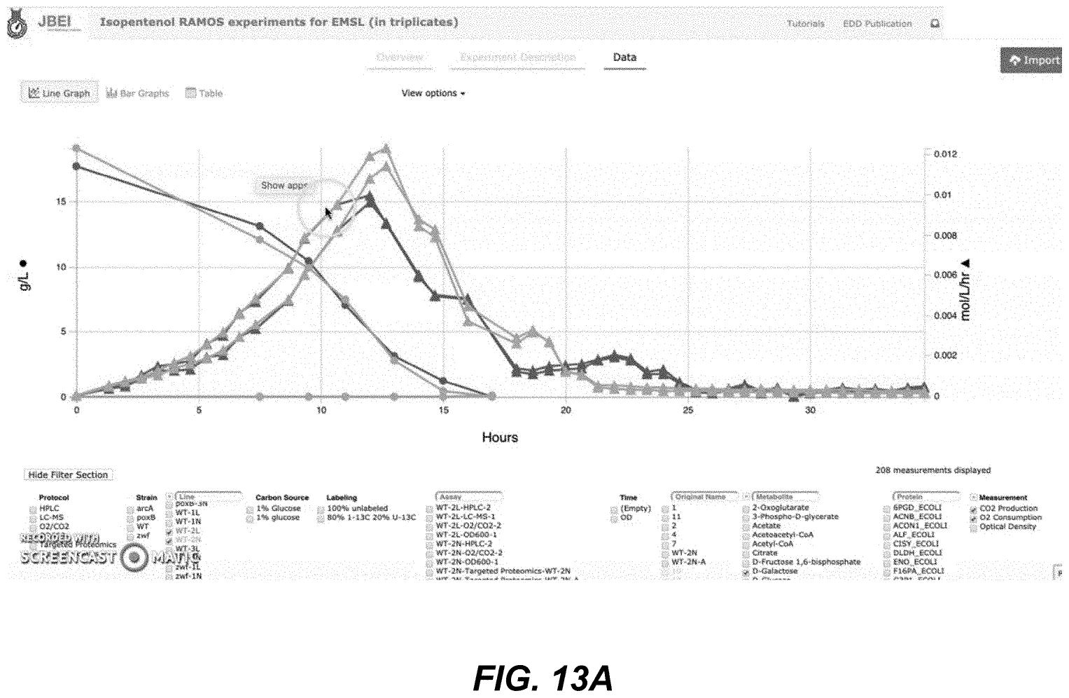

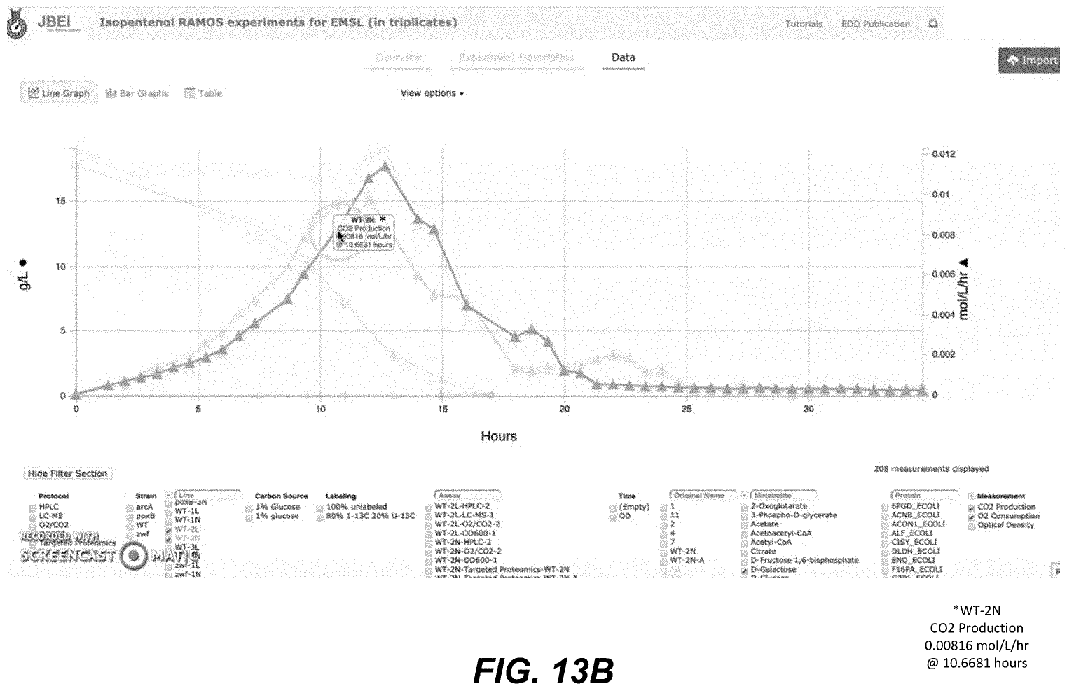

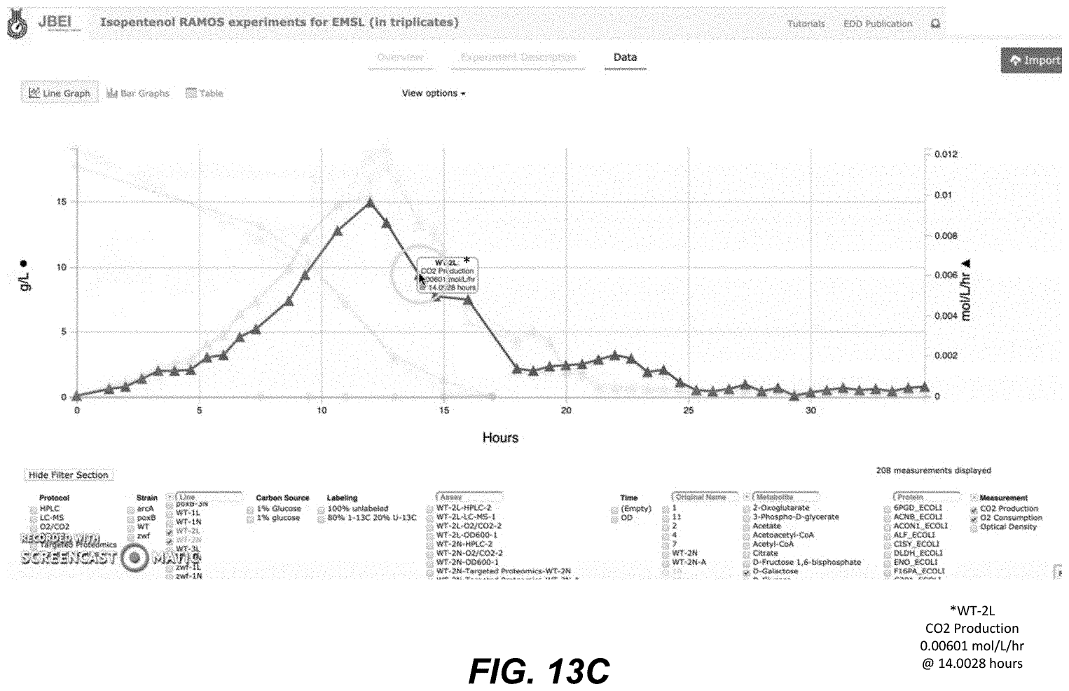

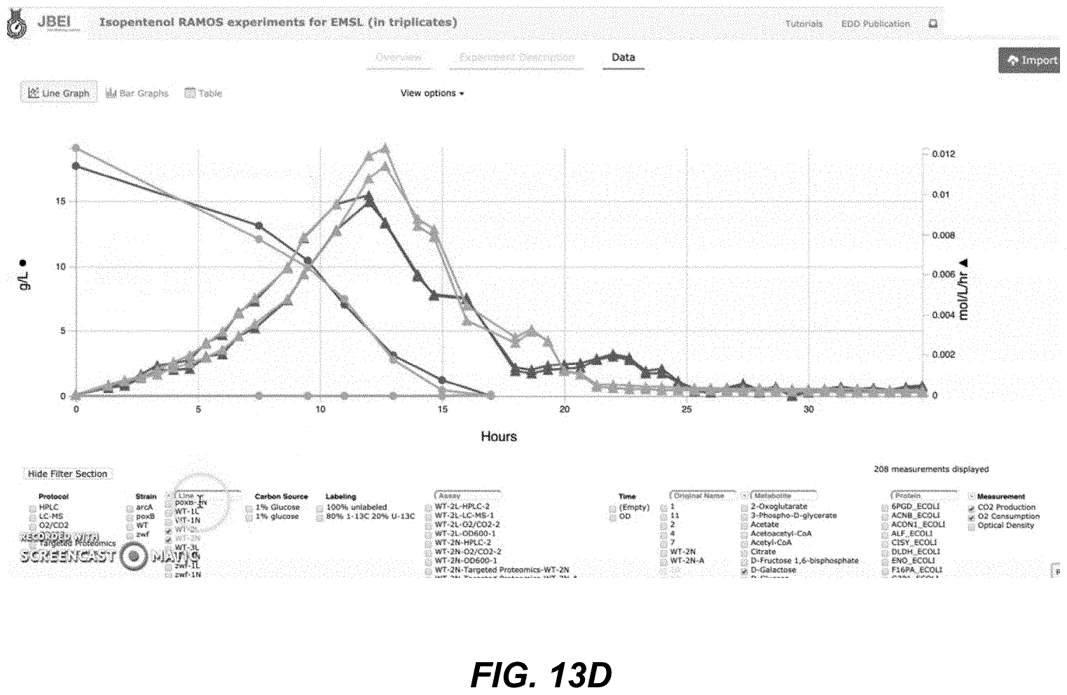

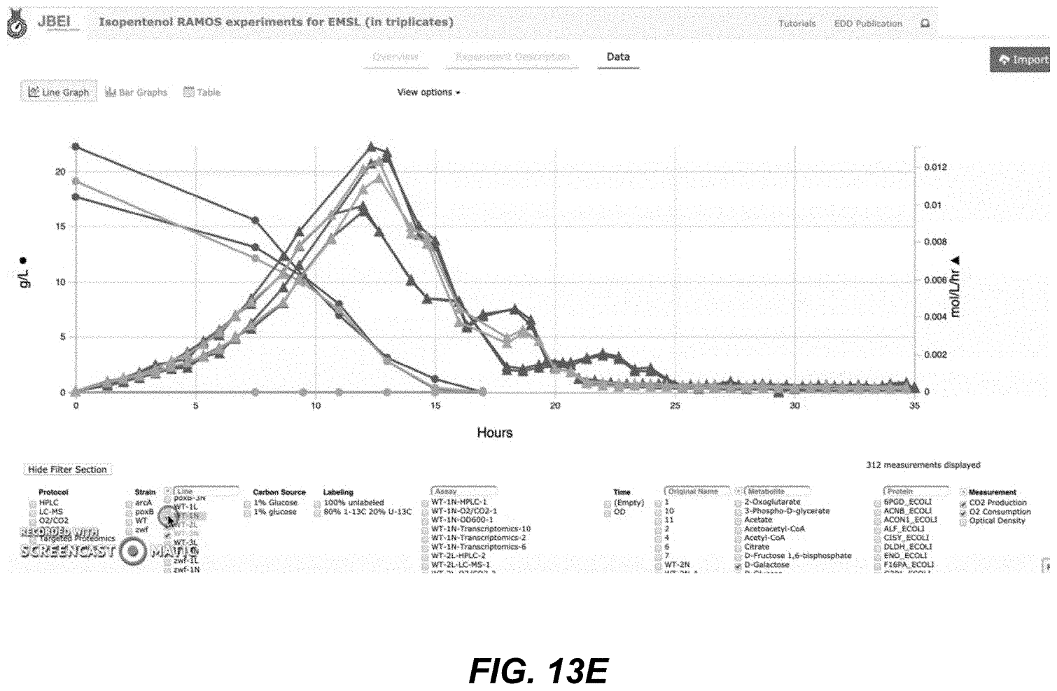

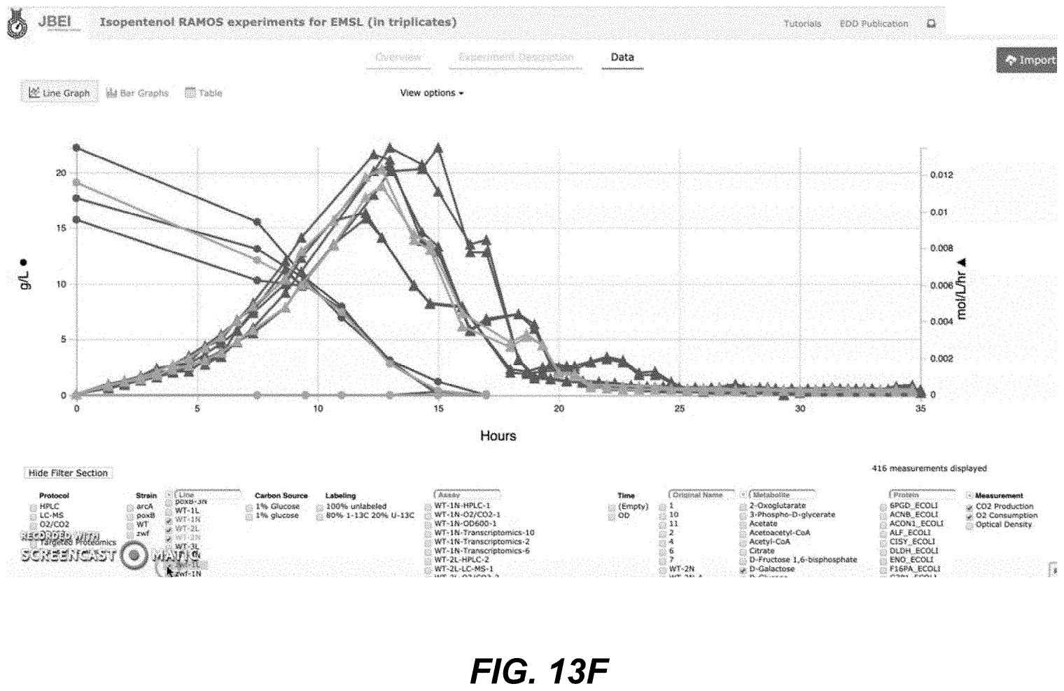

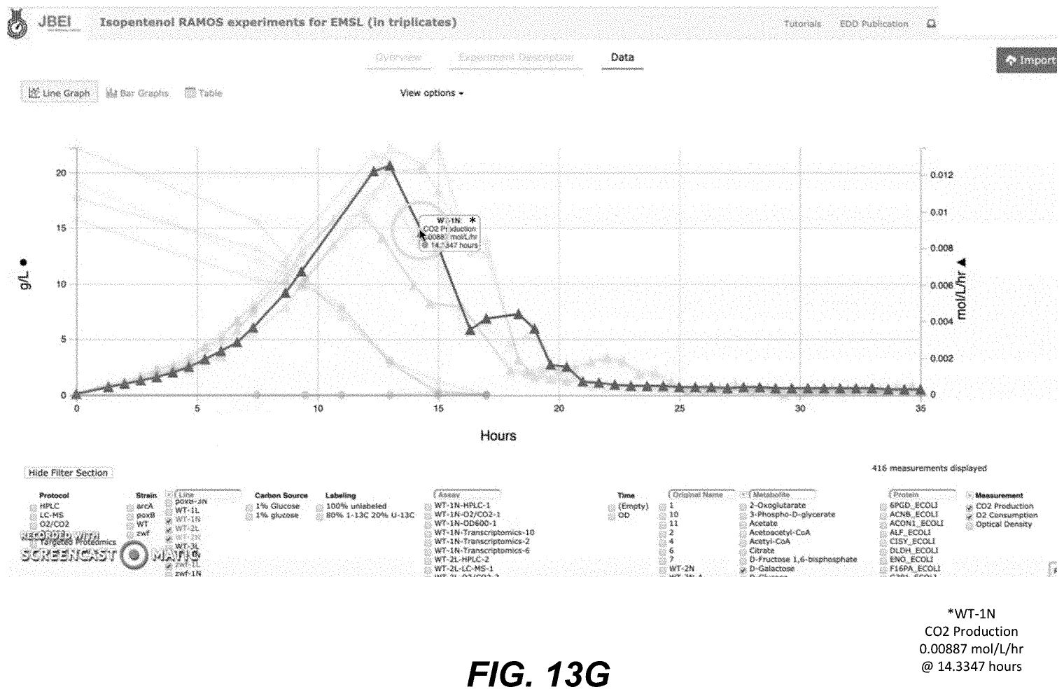

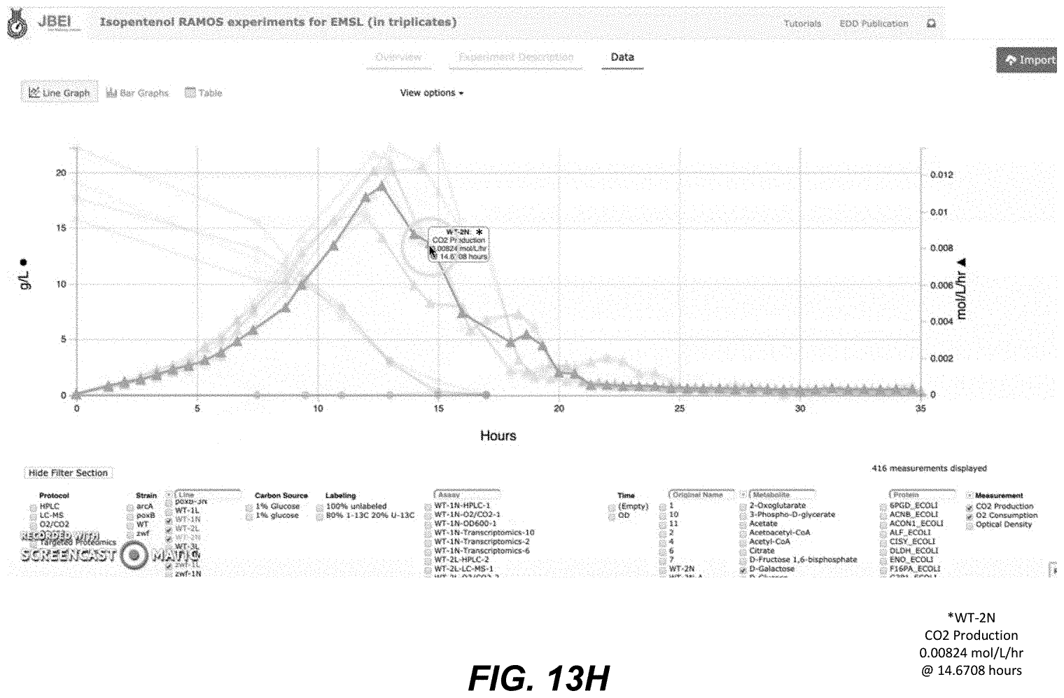

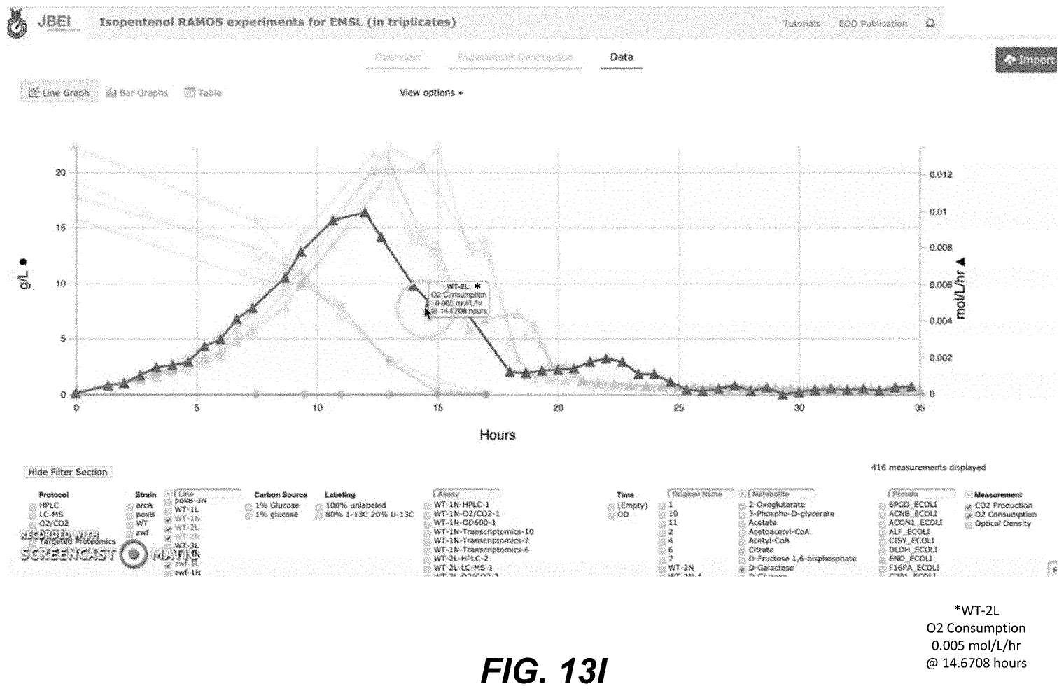

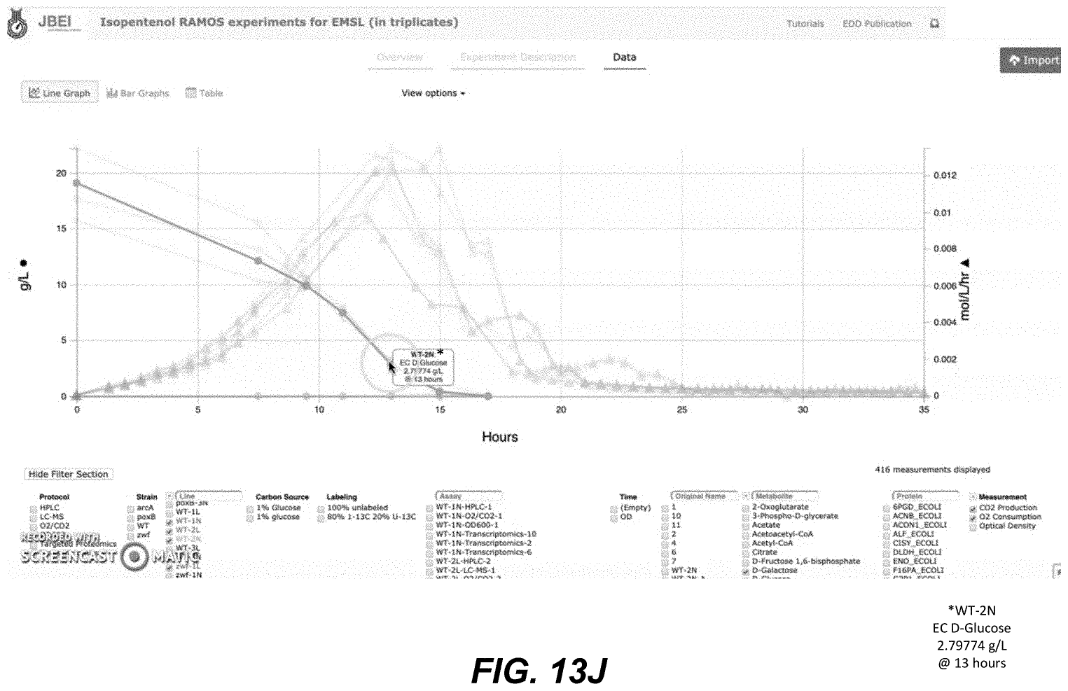

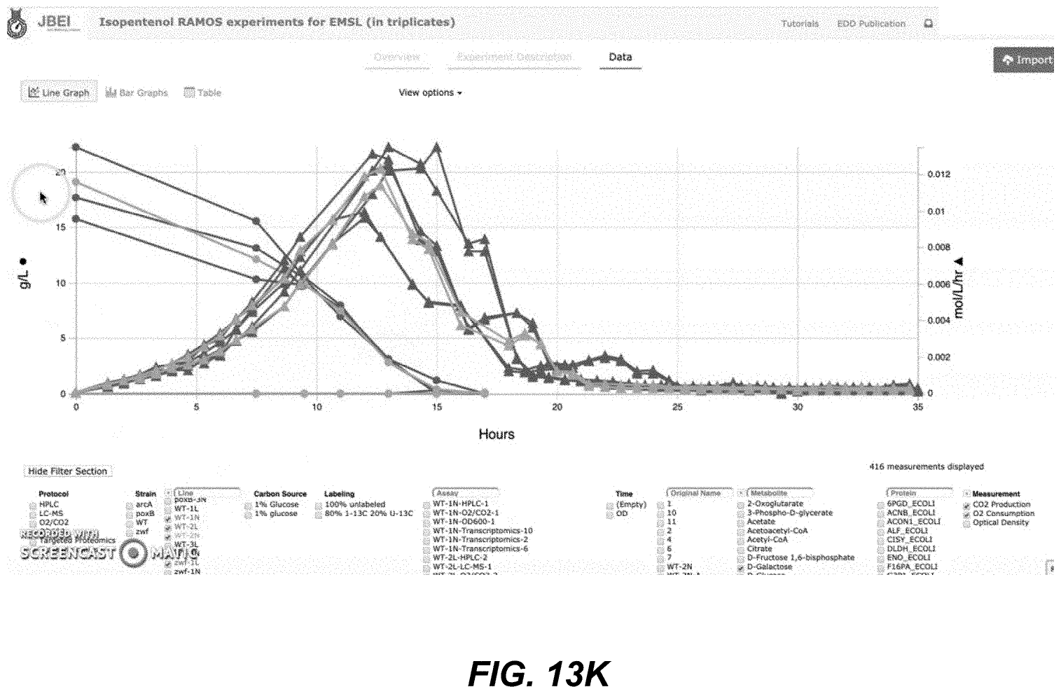

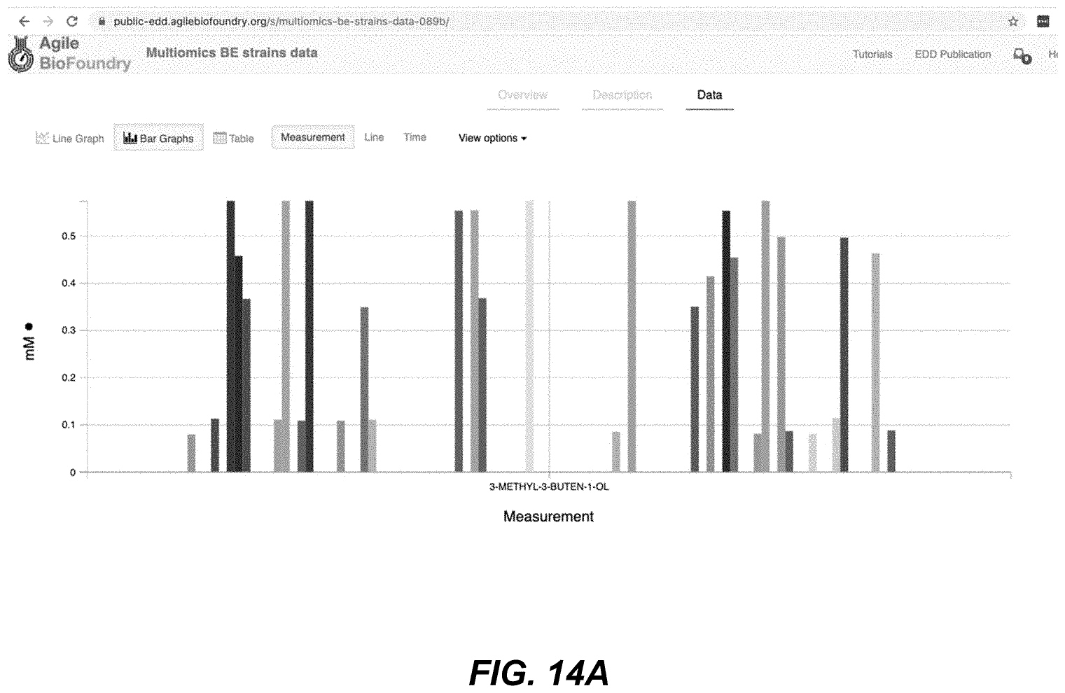

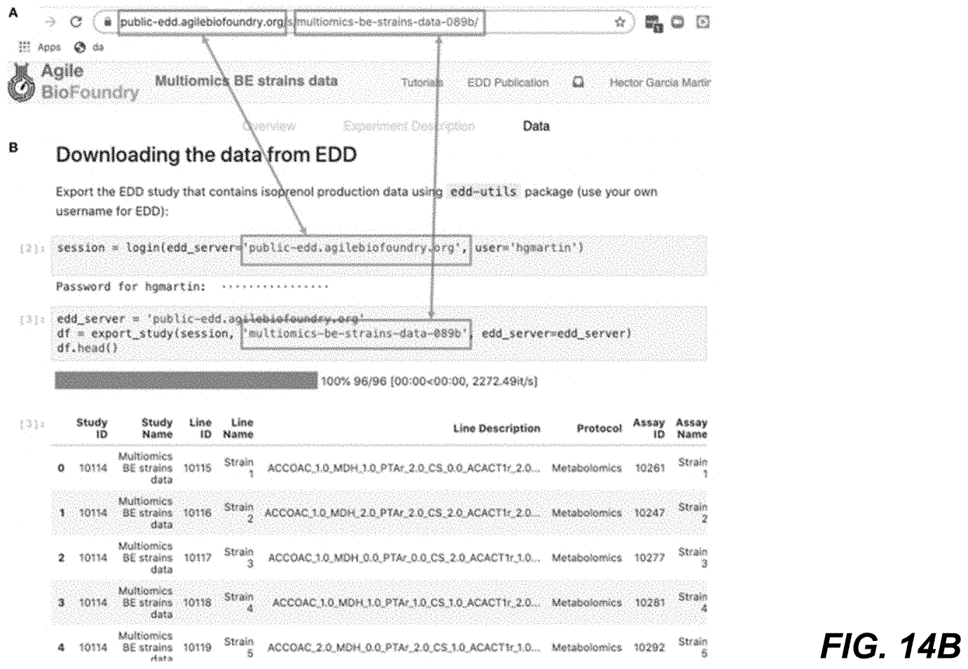

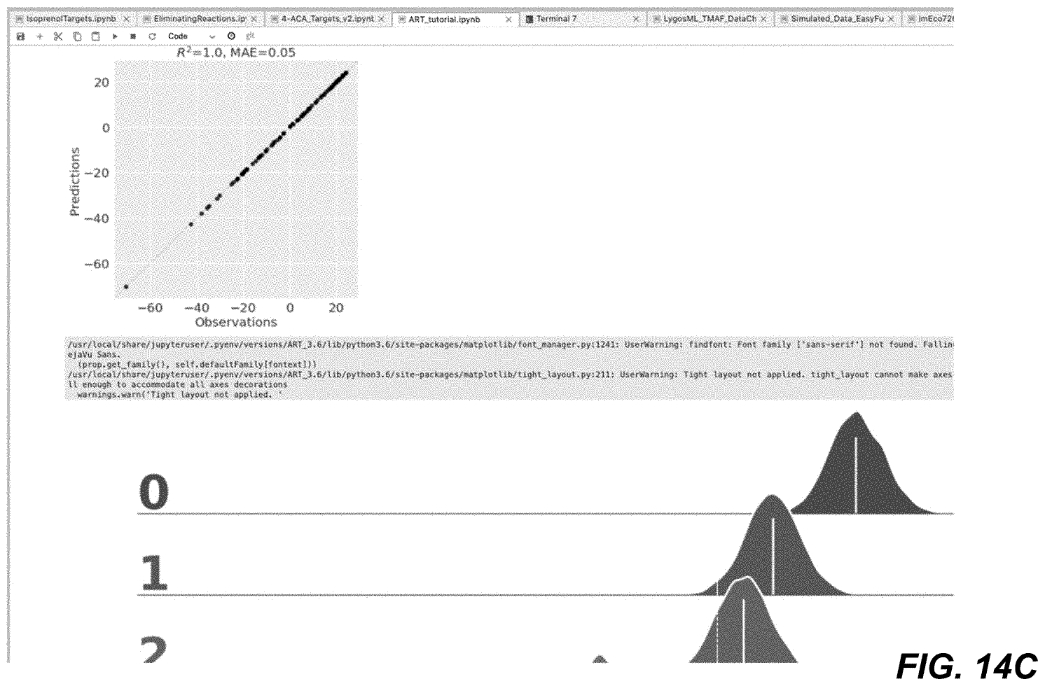

In some embodiments, such large amounts of data require dedicated infrastructure, for example, when collecting 80,000 data points per DBTL cycle, excel sheets are just not practical. The Experiment Data Depot (EDD) as shown in FIG. 11-FIG. 12 can store data in a standardized manner. EDD provides interactive visualization (FIG. 13A-FIG. 13K), and a user can easily download the data into, e.g., a Jupyter Notebook through the REST API (FIG. 14A-FIG. 14C).

In some embodiments, the Deep learning model requires good data quality check and allows for a different kinetic learning. In some embodiments, data checking and preprocessing is critical for downstream analysis. The checking and preprocessing can comprise: basic inspection of the exported dataframe, version control checks for each protocol (e.g., new data points in the last release, old data points not in the current release, different values between last and current release), basic data integrity checks that can be corrected where needed (e.g., formal vs measurement type, units, negative values, NaN values, replicates, missing data per protocol, replicate, time point), time evolution checks, duplicates checks, and TMAF monotonicity check, generation of files for EDD import of curated study (e.g., experiment description file, protocol files), variability analysis for technical replicates (e.g., coefficient of variation). Data curation can include: populating all units, populating formal types, setting negative values to zero, and/or removing strains with no data.

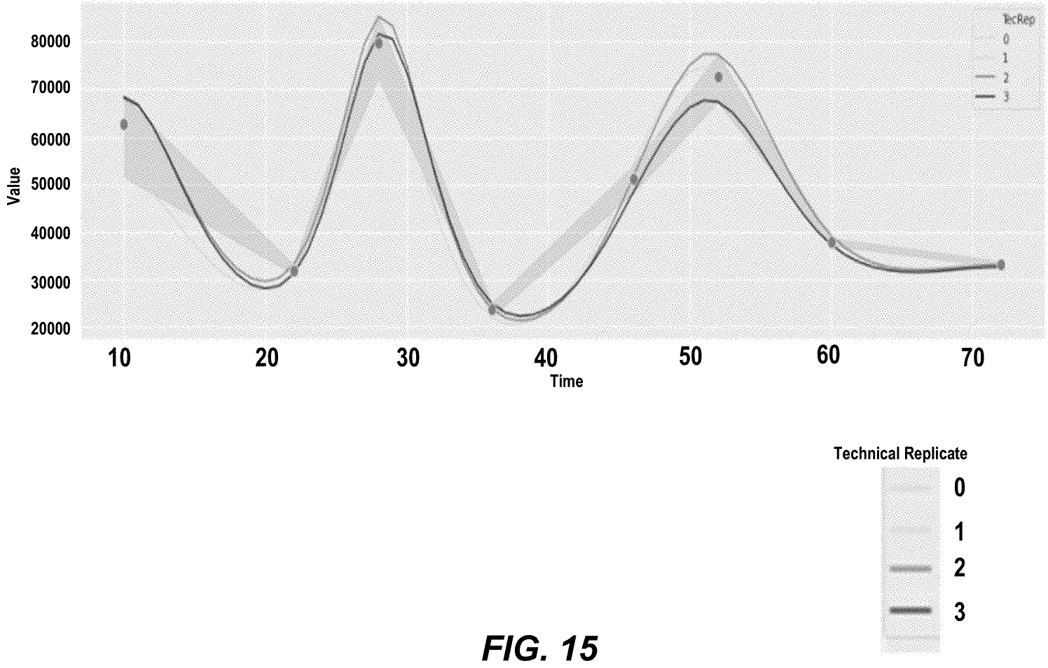

In some embodiments, model fitting requires smooth time series. Shown in FIG. 15 is a model fitting for 8 time points interpolated to 63 hourly time points using cubic spline method.

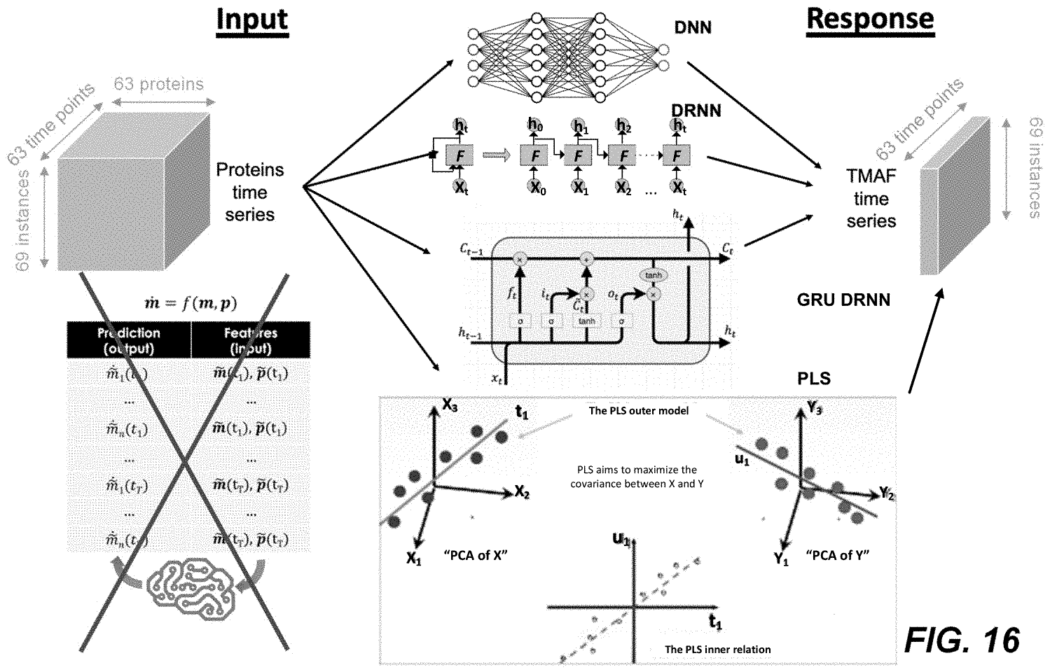

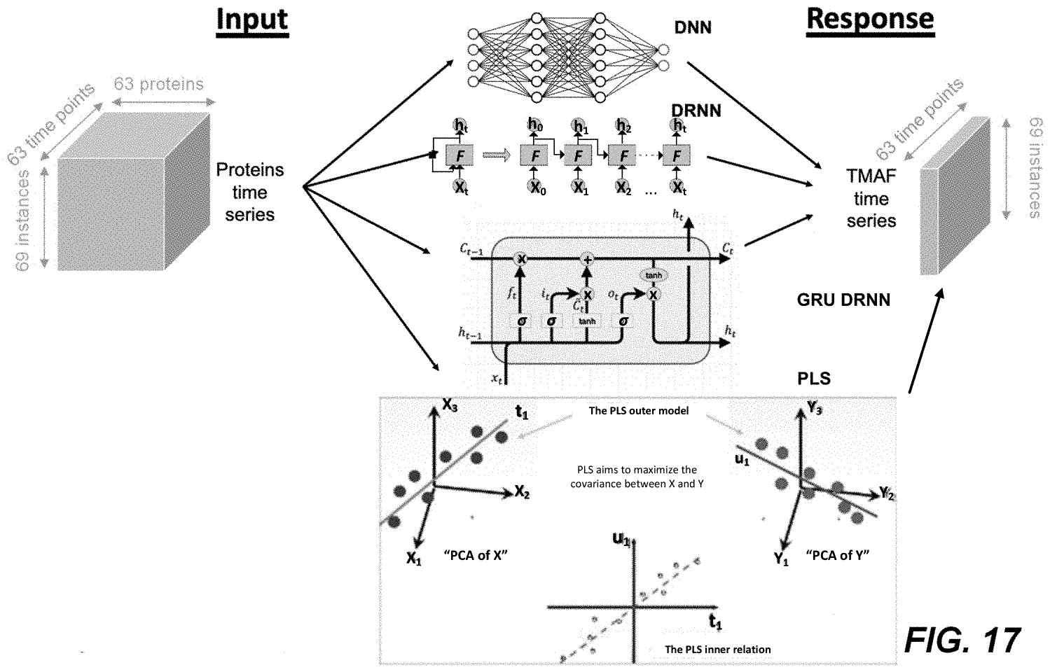

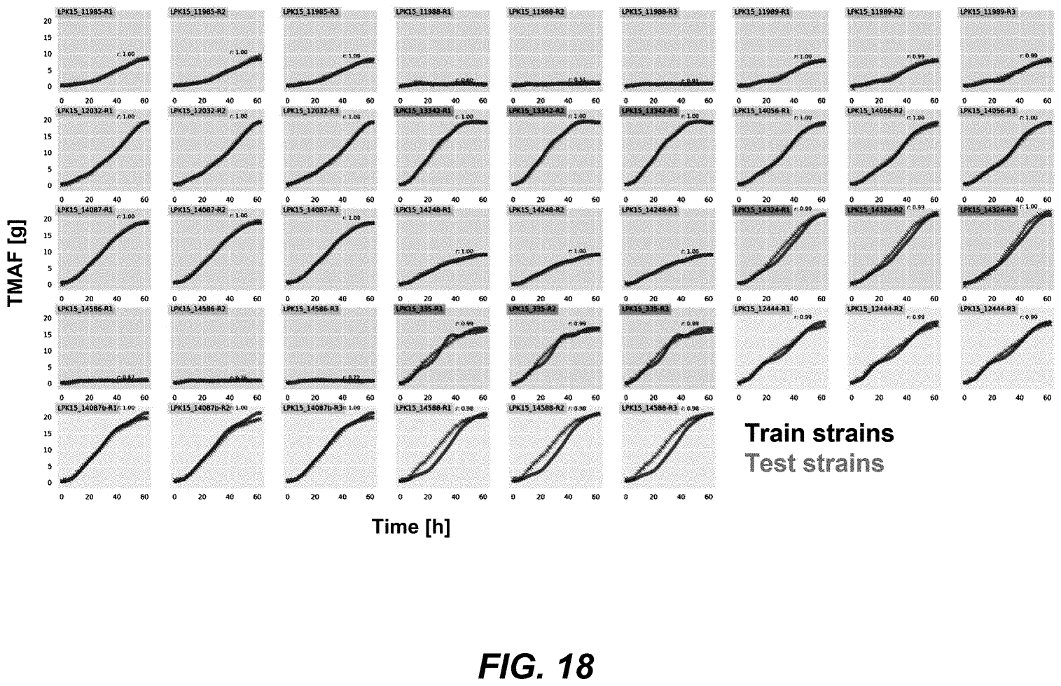

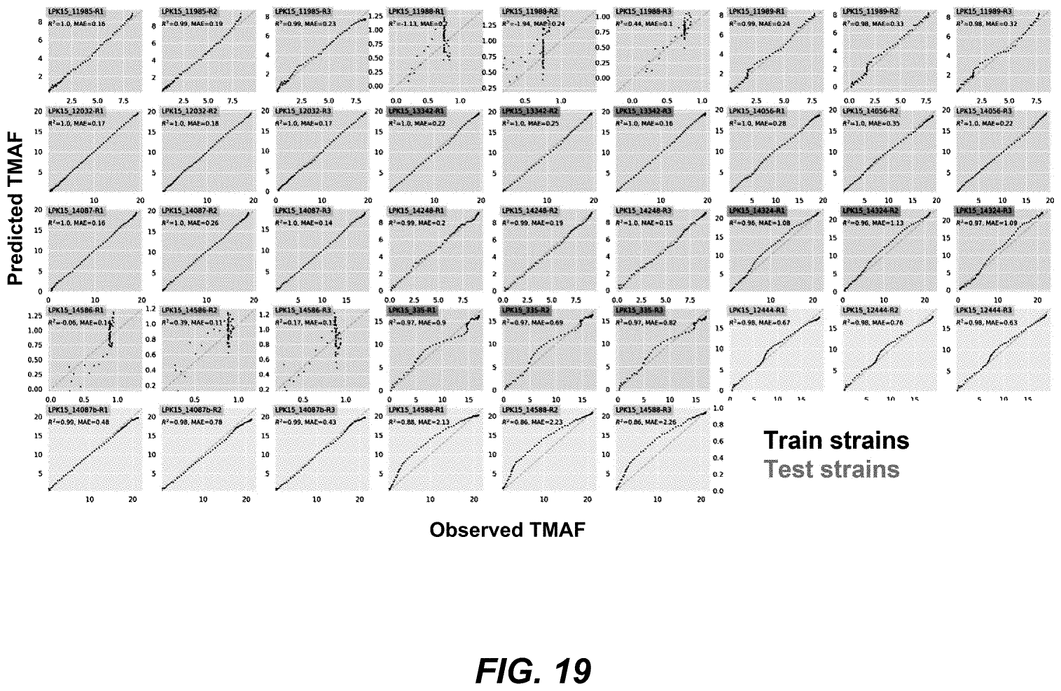

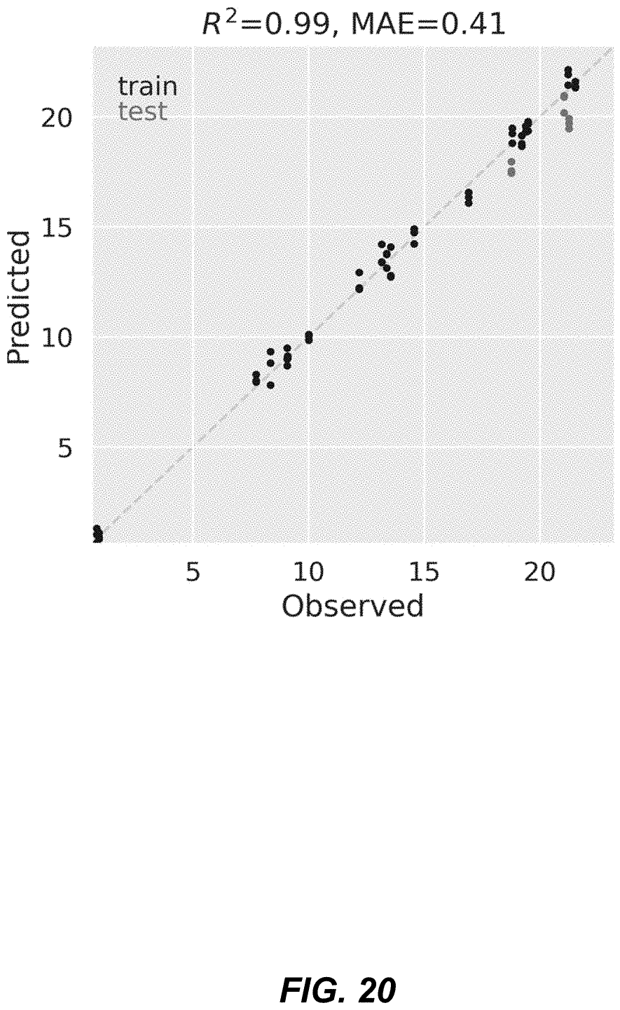

With Kinetic learning, response timelines can be predicted from input timelines, rather than derivatives (FIG. 16). Kinetic learning can utilize one or more machine learning models (e.g., an ensemble of machine learning models). For example, three Deep Neural Network (DNN) models form the final ensemble model and Partial Least Square (PLS) model can be used to prioritize protein for producing recommendations (FIG. 17). In some embodiments, the ensemble model is able to predict product dynamics (FIG. 18). The ensemble model shows agreement between predictions vs observations (FIG. 19), and the ensemble model accurately predicts the last time point for total malonic acid formed (TMAF) (FIG. 20).

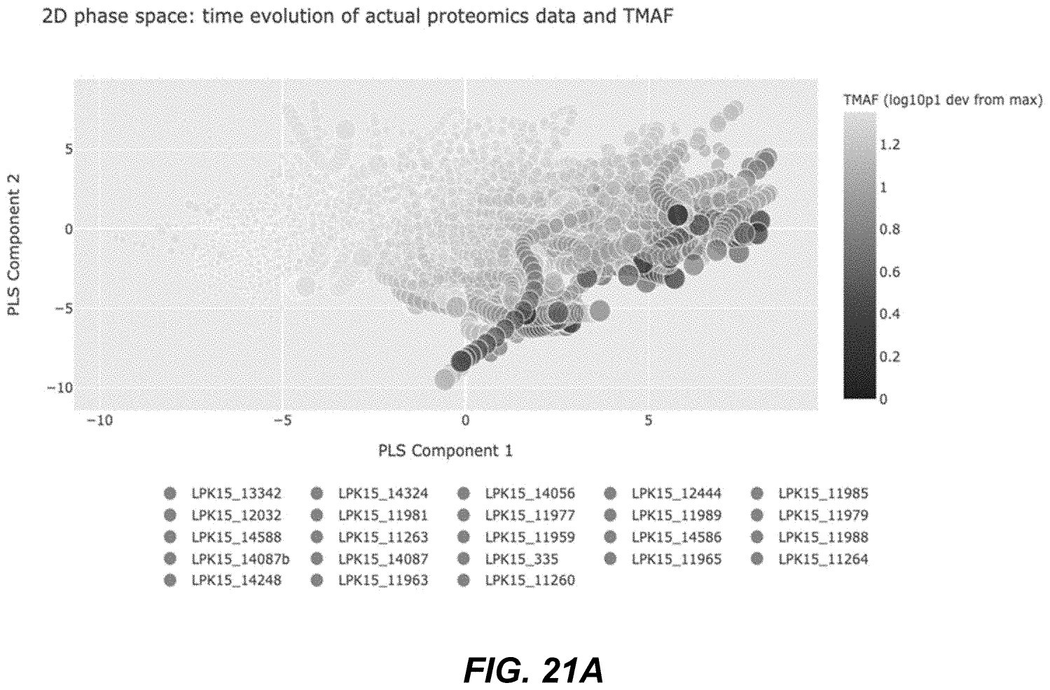

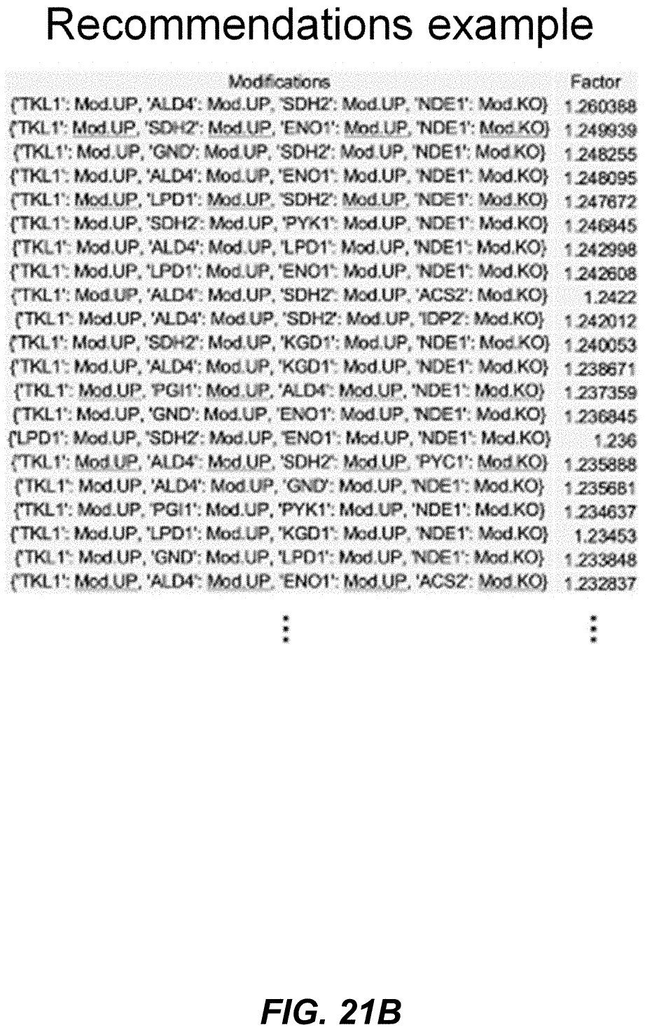

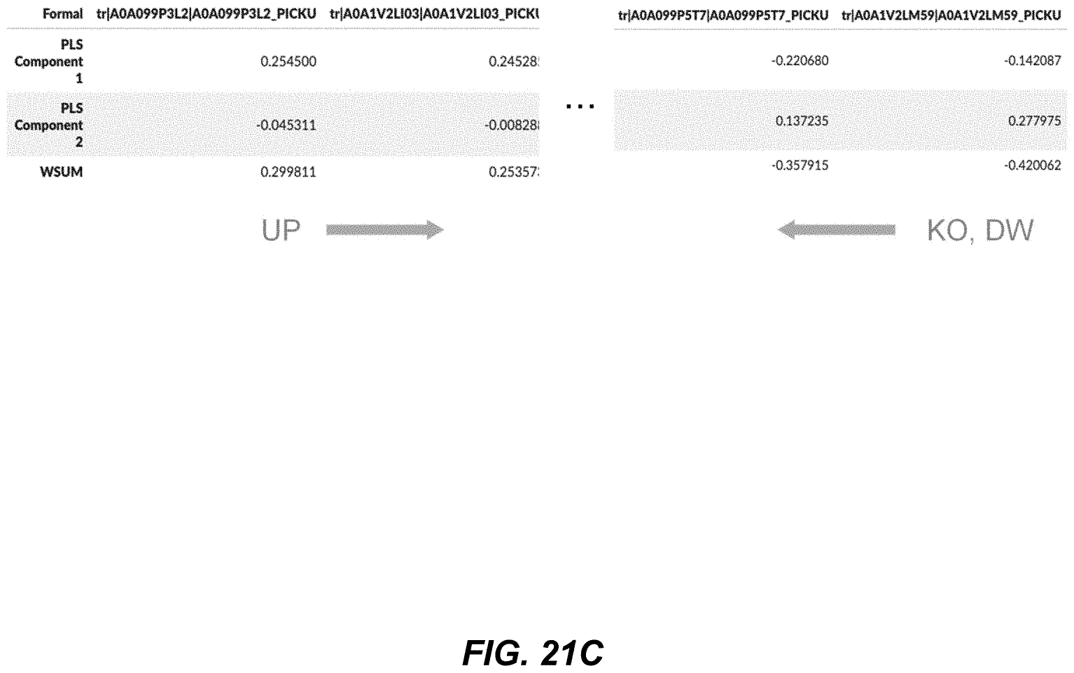

In some embodiments, recommendations are generated by exploring allowable modifications in protein space. Modifications of protein expressions include, but are not limited to: (1) ‘Up’ (increased expression): up to 3 protein, (2) ‘Knock-out’ (KO): 1 protein (in combination with max of 3 Ups), (3) ‘Down’ (DW) (reduced expression): 1 protein (in combination with max of 2 Ups). This can translate to, in some embodiments, 10 types of modifications (UP=2, KO=0, DW=0.5): (1) [DW], (2) [KO], (3) [UP], (4) [UP, DW], (5) [UP, KO], (6) [UP, UP], (7) [UP, UP, DW], (8) [UP, UP, KO], (9) [UP, UP, UP], (10) [UP, UP, UP, KO]. In some embodiments, an assumption is that modifications at initial time point are propagated in time at the same rate. In some embodiments, PLS are used to guide the exploration of possible modifications and make recommendations (FIG. 21A-FIG. 21B).

Described herein is, kinetic learning, which is purely data driven. A very large data set of, e.g., 480,000 data points, can be produced to train a model as disclosed herein for superior predictions using neural networks.

### Machine Learning Model

Non-limiting examples of machine learning models includes scale-invariant feature transform (SIFT), speeded up robust features (SURF), oriented FAST and rotated BRIEF (ORB), binary robust invariant scalable keypoints (BRISK), fast retina keypoint (FREAK), Viola-Jones algorithm, Eigenfaces approach, Lucas-Kanade algorithm, Horn-Schunk algorithm, Mean-shift algorithm, visual simultaneous location and mapping (vSLAM) techniques, a sequential Bayesian estimator (e.g., Kalman filter, extended Kalman filter, etc.), bundle adjustment, adaptive thresholding (and other thresholding techniques), Iterative Closest Point (ICP), Semi Global Matching (SGM), Semi Global Block Matching (SGBM), Feature Point Histograms, various machine learning algorithms (such as e.g., support vector machine, k-nearest neighbors algorithm, Naive Bayes, neural network (including convolutional or deep neural networks), or other supervised/unsupervised models, etc.), and so forth.

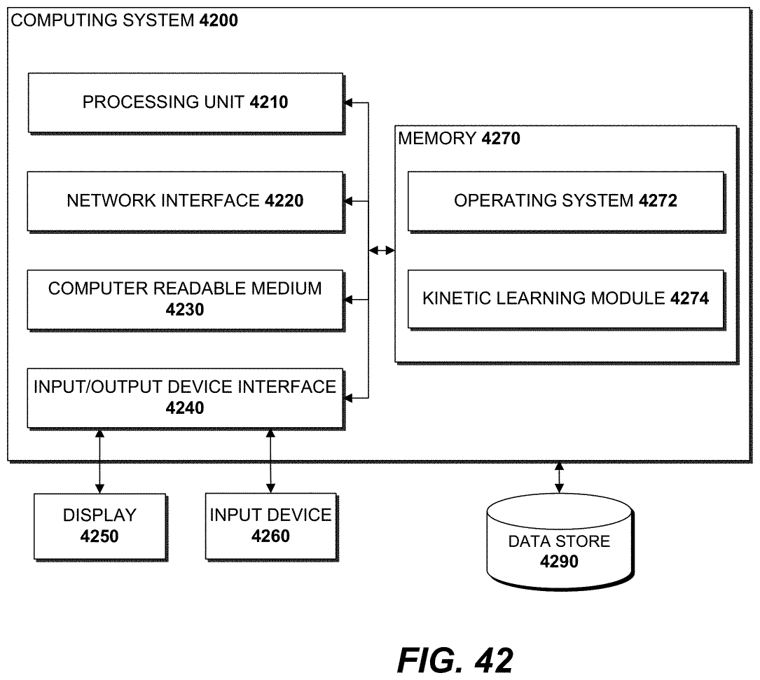

Once trained, a machine learning model can be stored in a computing system (e.g., the computing system 4200 described with reference to FIG. 42). Some examples of machine learning models can include supervised or non-supervised machine learning, including regression models (such as, for example, Ordinary Least Squares Regression), instance-based models (such as, for example, Learning Vector Quantization), decision tree models (such as, for example, classification and regression trees), Bayesian models (such as, for example, Naive Bayes), clustering models (such as, for example, k-means clustering), association rule learning models (such as, for example, a-priori models), artificial neural network models (such as, for example, Perceptron), deep learning models (such as, for example, Deep Boltzmann Machine, or deep neural network), dimensionality reduction models (such as, for example, Principal Component Analysis), ensemble models (such as, for example, Stacked Generalization), and/or other machine learning models.

A layer of a neural network (NN), such as a deep neural network (DNN) can apply a linear or non-linear transformation to its input to generate its output. A neural network layer can be a normalization layer, a convolutional layer, a softsign layer, a rectified linear layer, a concatenation layer, a pooling layer, a recurrent layer, an inception-like layer, or any combination thereof. The normalization layer can normalize the brightness of its input to generate its output with, for example, L2 normalization. The normalization layer can, for example, normalize the brightness of a plurality of images with respect to one another at once to generate a plurality of normalized images as its output. Non-limiting examples of methods for normalizing brightness include local contrast normalization (LCN) or local response normalization (LRN). Local contrast normalization can normalize the contrast of an image non-linearly by normalizing local regions of the image on a per pixel basis to have a mean of zero and a variance of one (or other values of mean and variance). Local response normalization can normalize an image over local input regions to have a mean of zero and a variance of one (or other values of mean and variance). The normalization layer may speed up the training process.

A convolutional neural network (CNN) can be a NN with one or more convolutional layers, such as, 5, 6, 7, 8, 9, 10, or more. The convolutional layer can apply a set of kernels that convolve its input to generate its output. The softsign layer can apply a softsign function to its input. The softsign function (softsign(x)) can be, for example, (x/(1+|x|)). The softsign layer may neglect impact of per-element outliers. The rectified linear layer can be a rectified linear layer unit (ReLU) or a parameterized rectified linear layer unit (PReLU). The ReLU layer can apply a ReLU function to its input to generate its output. The ReLU function ReLU(x) can be, for example, max(0, x). The PReLU layer can apply a PReLU function to its input to generate its output. The PReLU function PReLU(x) can be, for example, x if x>0 and ax if x<0, where a is a positive number. The concatenation layer can concatenate its input to generate its output. For example, the concatenation layer can concatenate four 5×5 images to generate one 20×20 image. The pooling layer can apply a pooling function which down samples its input to generate its output. For example, the pooling layer can down sample a 20×20 image into a 10×10 image. Non-limiting examples of the pooling function include maximum pooling, average pooling, or minimum pooling.

At a time point t, the recurrent layer can compute a hidden state s(t), and a recurrent connection can provide the hidden state s(t) at time t to the recurrent layer as an input at a subsequent time point t+1. The recurrent layer can compute its output at time t+1 based on the hidden state s(t) at time t. For example, the recurrent layer can apply the softsign function to the hidden state s(t) at time t to compute its output at time t+1. The hidden state of the recurrent layer at time t+1 has as its input the hidden state s(t) of the recurrent layer at time t. The recurrent layer can compute the hidden state s(t+1) by applying, for example, a ReLU function to its input. The inception-like layer can include one or more of the normalization layer, the convolutional layer, the softsign layer, the rectified linear layer such as the ReLU layer and the PReLU layer, the concatenation layer, the pooling layer, or any combination thereof.

The number of layers in the NN can be different in different implementations. For example, the number of layers in a NN can be 10, 20, 30, 40, or more. For example, the number of layers in the DNN can be 50, 100, 200, or more. The input type of a deep neural network layer can be different in different implementations. For example, a layer can receive the outputs of a number of layers as its input. The input of a layer can include the outputs of five layers. As another example, the input of a layer can include 1% of the layers of the NN. The output of a layer can be the inputs of a number of layers. For example, the output of a layer can be used as the inputs of five layers. As another example, the output of a layer can be used as the inputs of 1% of the layers of the NN.

The input size or the output size of a layer can be quite large. The input size or the output size of a layer can be n x m, where n denotes the width and m denotes the height of the input or the output. For example, n or m can be 11, 21, 31, or more. The channel sizes of the input or the output of a layer can be different in different implementations. For example, the channel size of the input or the output of a layer can be 4, 16, 32, 64, 128, or more. The kernel size of a layer can be different in different implementations. For example, the kernel size can be n x m, where n denotes the width and m denotes the height of the kernel. For example, n or m can be 5, 7, 9, or more. The stride size of a layer can be different in different implementations. For example, the stride size of a deep neural network layer can be 3, 5, 7 or more.

In some embodiments, a NN can refer to a plurality of NNs that together compute an output of the NN. Different NNs of the plurality of NNs can be trained for different tasks. A processor (e.g., a processor of the computing system 4200 descried with reference to FIG. 42) can compute outputs of NNs of the plurality of NNs to determine an output of the NN. For example, an output of a NN of the plurality of NNs can include a likelihood score. The processor can determine the output of the NN including the plurality of NNs based on the likelihood scores of the outputs of different NNs of the plurality of NNs.

### Guiding Synthetic Biology Via Machine Learning and Multi-Omics Technologies

Synthetic biology needs predictive power to enhance its global impact. Provided herein are tools that leverage machine learning to predict responses (e.g., production) and suggest next steps. The Automated Recommendation Tool (ART) described herein can be used to design pathways and media compositions for a variety of organisms and target molecules. ART was successfully used to design pathway for, e.g., tryptophan production. Also provided herein is, kinetic learning, which is purely data driven, and a very large data set of 480,000 data points can be produced to train it.

As described herein, predictions using neural networks have been successful. The methods provided herein advantageously leverage the increasing amounts of data available in modern synthetic biology. The method disclosed herein takes a purely data-driven approach, and does not require a deep knowledge of the pathway and final product. This advantageously provides a general method applicable to any host, pathway or metabolite. As described herein, pipelines are developed for data preprocessing, training multiple neural networks able to predict product dynamics, generating actionable recommendations predicted to improve production of a molecule, e.g., malonic acid. The methods disclosed herein can fulfill an important need as the collection costs for multi-omics data drops. Automated Recommendation Tool (ART).

Provided below are exemplary applications and uses of the Automated Recommendation Tool (ART) described herein. Multi-omics data sets are generated and leveraged to train machine learning models to make predictions on how to engineer, e.g., Pichia kudriavzevii strains to improve malonic acid production. The goal of the project is to improve production of malonic acid through multiple Design, Build, Test, Learn (DBTL) cycles.

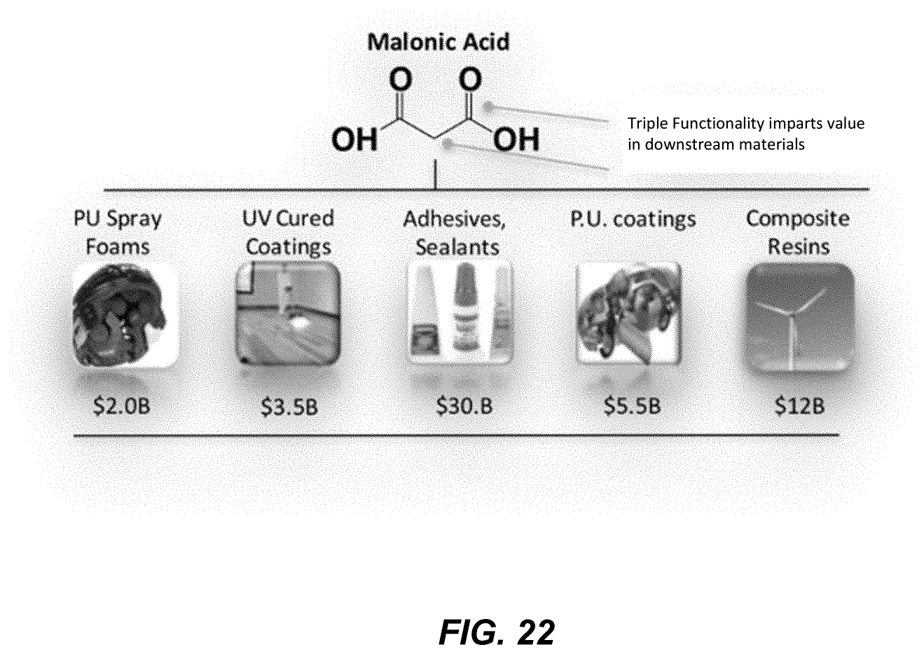

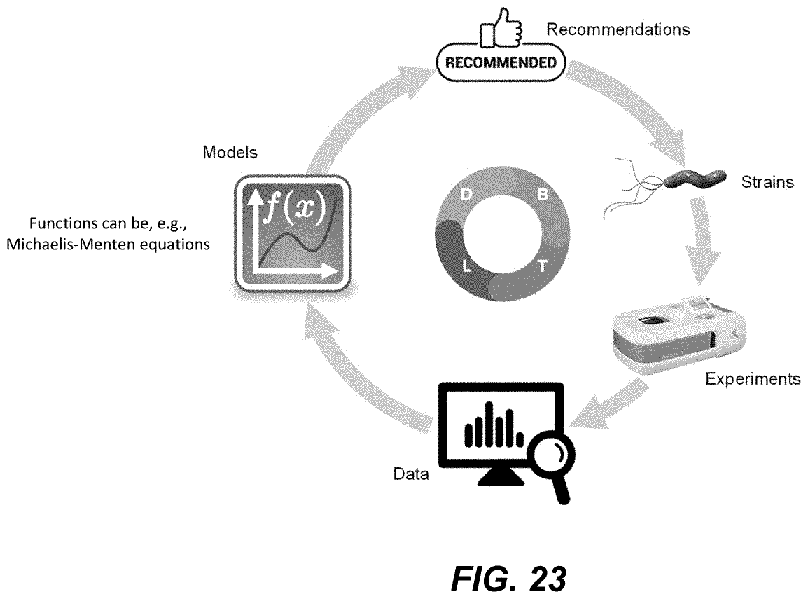

Malonic acid has been used for over 150-years in synthetic chemistry. However, it is difficult to produce from petrochemistry (<75% yields) and production is largely driven by foreign suppliers (FIG. 22). Kinetic modeling (differential equations-based kinetic modeling) can be used to predict metabolic behavior to produce a desired outcome. However, when predictions fail, it becomes complicated to systematically change the equations (FIG. 23). The methods disclosed herein can utilize machine learning to learn and predict kinetics. Machine learning can improve performance as more data is added (FIG. 2).

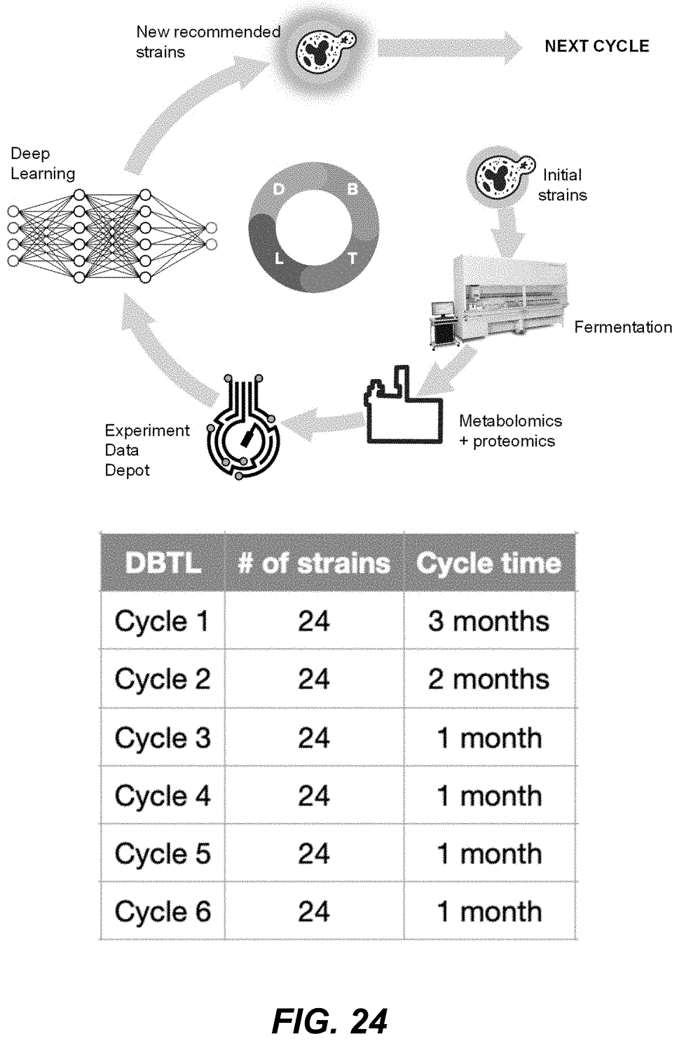

In some embodiments, each DBTL cycle produces a new set of 24 strains to be improved in the next cycles. 6 DBTL cycles in total can be performed for gathering the largest public multi-omics data set as compared to previous methods (FIG. 24). Multi-omics time-series of 80,000 data points per DBTL cycle can be produced. The multi-omics data set can include: Proteomics (63 proteins), Metabolomics (72 metabolites), HPLC (11 metabolites), Bioreactor (6 measurements—Total Malonic Acid Formed (TMAF), pH, dry cell weight (DCW), Dissolved Oxygen (DO), CO2, O2), OD600, 24 strains, 3 replicates, and 8-point time-series (FIG. 10). This is the largest dataset (containing real data) that has ever been employed for this sort of machine learning and strain improvement (as compared to, e.g., previous methods). Both intra- and extracellular metabolomics can be predicted.

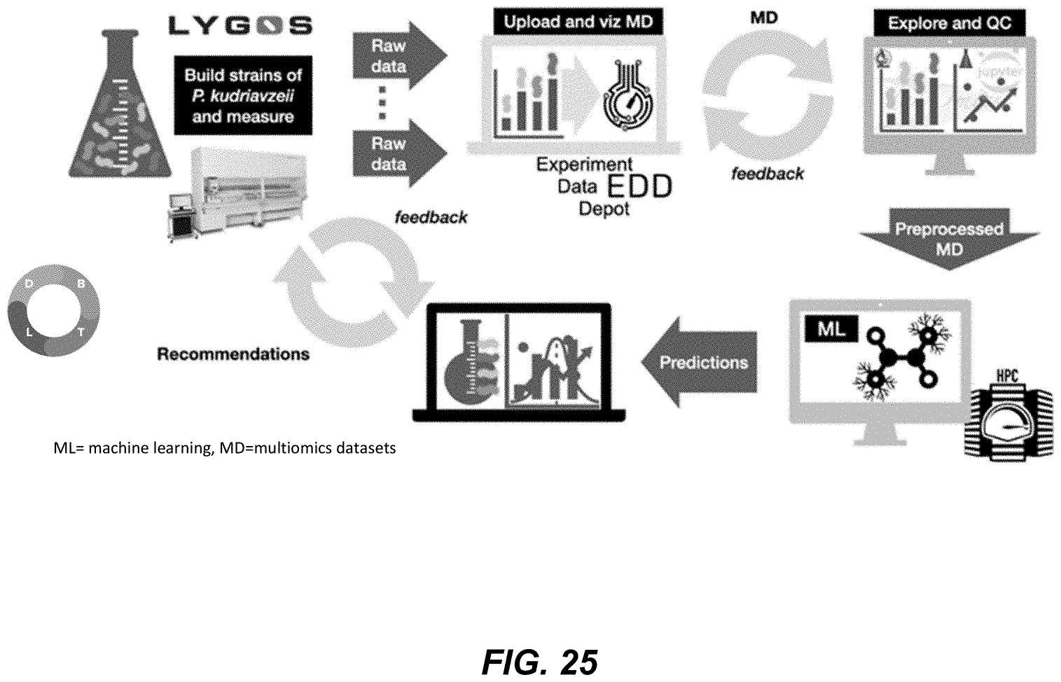

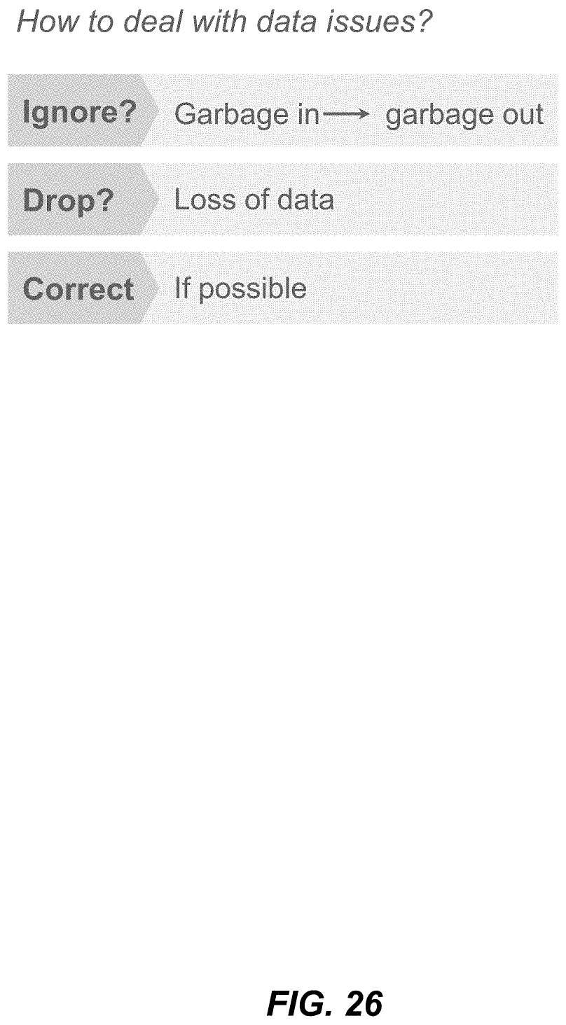

An exemplary machine learning (ML) workflow with multi-omics data is shown in FIG. 25. In some embodiments, high quality data with relatively low variation is needed to ensure confidence in the recommendations (FIG. 26). In some embodiments, data quality (checking and preprocessing) is critical for downstream analysis. In some embodiments, model fitting requires thorough data preparation includes: prepare & check data sets for ML, data interpolation (e.g., a number of data points interpolated to more data points, such as 8 time points interpolated to 63 hourly time points), define training/test strains, define input and response, data set standardization, and data set train/validation partitioning. A model of 8 time points interpolated to 63 hourly time points is shown in FIG. 15. For this exemplary application for malonic acid, test strains chosen included: LPK15_14087b, should be similar to LPK15_14087, a good test for similar case. Others were chosen by looking into the experiment data depot (EDD), one with low, the other with medium level of malonate.

As shown in FIG. 17, response time-series can be predicted from input time-series. The three DNN models can form the final ensemble model and a PLS model can be used to prioritize protein for producing recommendations. Some artificial neural network (ANN) layers account for the core metabolism while other specific ANN layers focus on particular metabolic product.

As shown in FIG. 18, the ensemble model is able to predict product dynamics acceptably. Test strains choice included: LPK15_14087b, should be similar to LPK15_14087, a good test for similar case. Others were chosen by looking into EDD, one with low, the other with medium level of malonate. As shown in FIG. 19, the ensemble model shows good agreement of predictions vs observations, and the ensemble model is able to predict the last time point for TMAF (FIG. 20).

Recommendations can be generated by exploring allowable modifications in protein space. Modifications of protein expressions, for each strain, can comprise: (1) ‘Up’ (increased expression): up to 3 proteins, (2) ‘Knock-out’ (KO): 1 protein (in combination with max of 3 Ups), (3) ‘Down’ (DW) (reduced expression): 1 protein (in combination with max of 2 Ups). This can translate to, in some embodiments, 10 types of modifications (UP=2, KO=0, DW=0.5): (1) [DW], (2) [KO], (3) [UP], (4) [UP, DW], (5) [UP, KO], (6), [UP, UP], (7) [UP, UP, DW], (8) [UP, UP, KO], (9) [UP, UP, UP], (10) [UP, UP, UP, KO].

In some embodiments, Partial least squares (PLS) are used to guide the exploration of possible modifications and make recommendations and recommendations are sorted according to predicted response (FIG. 21A-FIG. 21C).

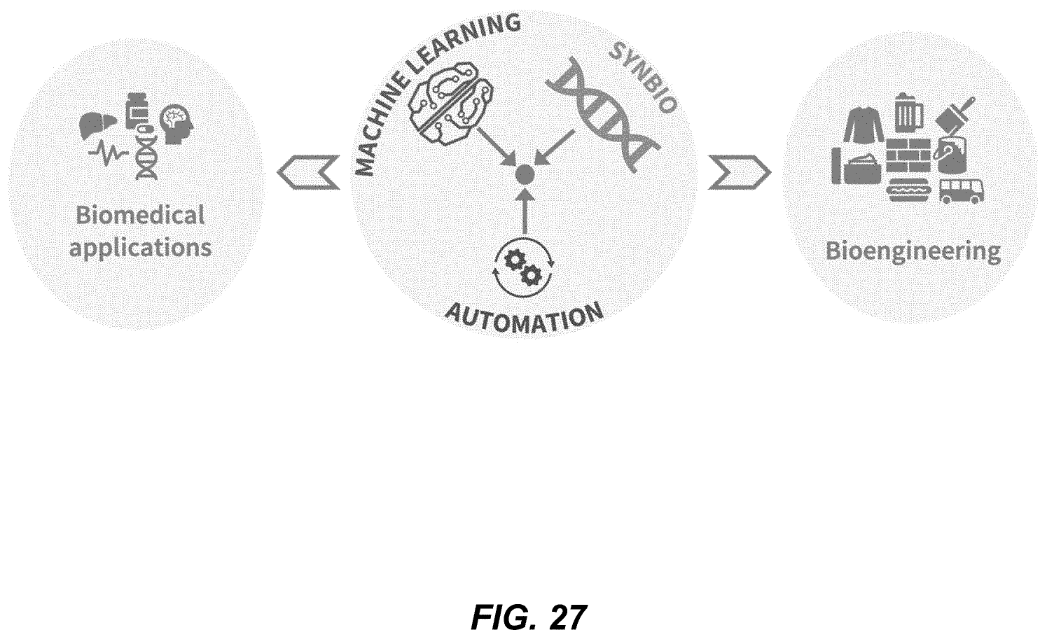

Synthetic biology needs predictive power to enhance its global impact. Described herein are tools that leverage machine learning to predict responses (e.g., production). ART can be successfully used to design pathway for, e.g., tryptophan production. ART can be used to design pathways and media compositions for a variety of organisms and target molecules. Also described herein is kinetic learning, which is purely data driven. A very large data set of ˜480,000 data points is produced to train it and make predictions using neural networks. As shown herein, Machine Learning (ML)+Synthetic Biology (SynBio)+Automation complement each other perfectly (FIG. 27). ML can provide predictive power but it needs large amounts of high-quality data. Automation can provide the data required by machine learning (robotic stations, microfluidics, cloud labs)—improving reliability and reproducible of results. As provided herein, ART is advantageously able to leverage the increasing amounts of data available in modern synthetic biology. ART leverages machine learning to predict responses (e.g., production) and suggest next steps.

### Simulating the Metabolic Pathway Dynamics of an Organism

Disclosed herein are systems and methods for determining metabolic pathway dynamics using time series multiomics data. In one example, after receiving time series multiomics data comprising time-series metabolomics data associated a metabolic pathway and time-series proteomics data associated with the metabolic pathway, derivatives of the time series multiomics data can be determined. A machine learning model, representing a metabolic pathway dynamics model, can be trained using the time series multiomics data and the derivatives of the time series multiomics data, wherein the metabolic pathway dynamics model relates the time-series metabolomics data and time-series proteomics data to the derivatives of the time series multiomics data. The method can include simulating a virtual strain of the organism using the metabolic pathway dynamics model.

Disclosed herein are systems and methods for determining metabolic pathway dynamics using time-series multiomics data. In one example, the method includes: receiving time-series multiomics data comprising time-series metabolomics data associated a metabolic pathway and time-series proteomics data associated with the metabolic pathway; determining derivatives of the time-series multiomics data; training a machine learning model, representing a metabolic pathway dynamics model, using the time-series multiomics data and the derivatives of the time-series multiomics data, wherein the metabolic pathway dynamics model relates the time-series metabolomics data and time-series proteomics data to the derivatives of the time-series multiomics data; and simulating a virtual strain of the organism using the metabolic pathway dynamics model.

In another example, the system includes: computer-readable memory storing executable instructions; and one or more hardware processors programmed by the executable instructions to perform a method comprising: receiving time-series multiomics data comprising time-series metabolomics data associated a metabolic pathway and time-series proteomics data associated with the metabolic pathway; determining derivatives of the time-series multiomics data; training a machine learning model, representing a metabolic pathway dynamics model, using the time-series multiomics data and the derivatives of the time-series multiomics data, wherein the metabolic pathway dynamics model relates the time-series metabolomics data and time-series proteomics data to the derivatives of the time-series multiomics data; and simulating a virtual strain of the organism using the metabolic pathway dynamics model.

Disclosed herein are systems and methods for simulating the pathway dynamics of a virtual strain of an organism. In one example, the method includes: receiving time-series multiomics data comprising a first time-series multiomics data associated a metabolic pathway and a second time-series multiomics data associated with the metabolic pathway; determining derivatives of the first time-series multiomics data; training a machine learning model, representing a metabolic pathway dynamics model, using the first time-series multiomics data, the derivatives of the first time-series multiomics data, and the second time-series multiomics data, wherein the metabolic pathway dynamics model relates the first time-series multiomics data and the second time-series multiomics data to the derivatives of the first time-series multiomics data; and simulating a virtual strain of the organism using the metabolic pathway dynamics model.

In another example, the system includes computer-readable memory storing executable instructions; and one or more hardware processors programmed by the executable instructions to perform a method comprising: receiving time-series multiomics data comprising time-series metabolomics data associated a metabolic pathway and time-series proteomics data associated with the metabolic pathway; determining derivatives of the time-series multiomics data; training a machine learning model, representing a metabolic pathway dynamics model, using the time-series multiomics data and the derivatives of the time-series multiomics data, wherein the metabolic pathway dynamics model relates the time-series metabolomics data and time-series proteomics data to the derivatives of the time-series multiomics data; and simulating a virtual strain of the organism using the metabolic pathway dynamics model.

Disclosed herein are systems and methods for accurately and efficiently determining dynamics of a metabolic pathway. In one embodiment, the metabolic pathway is a heterologous metabolic pathway. In one embodiment, the method comprises determining or inferring the dynamics of a metabolic pathway using time series proteomics and metabolomics data. The genomic and post-genomic revolutions have generated orders of magnitude more data than biological researchers can interpret, in the form of functional genomics data (transcriptomics, proteomics, metabolomics and fluxomics). One method described herein leverages these large sets of functional genomics data to predict metabolite concentration time series from the knowledge of protein levels.

The method can include determining a computational model of a particular organism based on the dynamics of one or more metabolic pathways in the organism using time-series data. In one embodiment, the model is not based on Michaelis-Menten kinetics which is based on a plurality of differential equations. The model may supplement, or complement, a model based on Michaelis-Menten kinetics. The model can be scalable to genome-scale time-series data. The model can be based on a plurality of relationships or expressed as a plurality of equations. The right hand side of the equation (see Eq. (3) below) can be estimated through machine learning methods as a function of metabolite and protein concentrations. In one implementation, the machine learning model can be a supervised machine learning model.

In one embodiment, the method comprises accurately determining or estimating time-series data that can be used to train a machine learning model with an accurate model performance. The amount of time-series data required for achieving good model performance can be estimated based on simulated data of one or more metabolic pathways. In one example, the simulated data is proteomics or metabolomics data, such as the mevalonate pathway engineered in E. coli.

In one embodiment, the method can include determining an amount of time-series data sufficient for determining an accurate model with predetermined accuracy. In one embodiment, the method can include evaluating the simulated data against real data for strains of an organism of interest. For example, the organism may be engineered to produce certain compounds, such as limonene, isopentenol, bisaboline, or organic molecules of interest. In one embodiment, the method comprises predicting production of a medium titer strain using time-series data for high and low producing strains as training sets. In one embodiment, the method comprises receiving or generating sufficient time-series data for determining the dynamics of complex coupled nonlinear systems relevant to metabolic engineering.

Disclosed herein include systems for simulating the pathway dynamics of a virtual strain of an organism. In some embodiments, a system for simulating the pathway dynamics of a virtual strain comprises computer-readable memory storing executable instructions; and one or more hardware processors. The hardware processors can be programmed by the executable instructions to perform: receiving time-series multiomics data of a plurality of strains of the organism, the times-series multiomics data comprising time-series metabolomics data and time-series proteomics data associated with a metabolic pathway. The hardware processors can be programmed by the executable instructions to perform: determining derivatives of the time-series metabolomics data. The hardware processors can be programmed by the executable instructions to perform: training a machine learning model, representing a metabolic pathway dynamics model, using the time-series multiomics data and the derivatives of the time-series metabolomics data, wherein the metabolic pathway dynamics model relates the time-series metabolomics data and time-series proteomics data to the derivatives of the time-series metabolomics data. The hardware processors can be programmed by the executable instructions to perform: simulating a virtual strain of the organism using the metabolic pathway dynamics model to determine a characteristics of a metabolic pathway represented by the metabolic pathway dynamics model in the virtual strain.

The hardware processors can be programmed by the executable instructions to perform: designing one or more new strains based on the virtual strain; generating experimental time-series multiomics data for the new strains; and retraining the machine learning model based on the experimental time-series multiomics data of the new strains.

The characteristic of the metabolic pathway can be a titer, rate, or yield of a product of the metabolic pathway. The time-series multiomics data can comprise time-series multiomics data of a plurality of strains of an organism. The metabolic pathway can comprise a heterologous pathway.

The machine learning model comprises a supervised machine learning model. The machine learning model can comprise a non-classification model, a neural network, a recurrent neural network (RNN), a linear regression model, a logistic regression model, a decision tree, a support vector machine, a Naïve Bayes network, a k-nearest neighbors (KNN) model, a k-means model, a random forest model, a multilayer perceptron, or a combination thereof. The machine learning model can comprise parameters representing kinetics of the metabolic pathway and parameters associated with the plurality of strains.

Training the machine learning model can comprises training the machine learning model using training data comprising triplets of a protein concentration, a metabolite concentration, and a metabolite derivative. Simulating the virtual strain of the organism can comprise integrating the metabolic pathway dynamics model over a time period of interest. Simulating the virtual strain of the organism can comprise determining a concentration of a metabolite of the metabolic pathway using the metabolic pathway dynamics model.

The one or more hardware processor can be programmed by the executable instructions to perform: smooth the time-series metabolomics data to generate smoothed time-series metabolomics data, wherein determining the derivatives of the time-series metabolomics data comprises determining derivatives of the smoothed time-series metabolomics data, and wherein training the machine learning model comprises training the machine learning model using the smooth time-series multiomics data and the derivatives of the smoothed metabolomics data. Smoothing the time-series metabolomics data can comprise smoothing the time-series metabolomics data using a filter. The filter can comprise a Savitzky-Golay filter.

Disclosed herein include methods for simulating the metabolic pathway dynamics of a strain of an organism. In some embodiments, a method for simulating the metabolic pathway dynamics of a strain of an organism, comprises: receiving time-series multiomics data comprising a first time-series multiomics data associated a metabolic pathway and a second time-series multiomics data associated with the metabolic pathway. The method can comprise: determining derivatives of the first time-series multiomics data. The method can comprise: training a machine learning model, representing a metabolic pathway dynamics model, using the first time-series multiomics data, the derivatives of the first time-series multiomics data, and the second time-series multiomics data, wherein the metabolic pathway dynamics model relates the first time-series multiomics data and the second time-series multiomics data to the derivatives of the first time-series multiomics data. The method can comprise: simulating a virtual strain of the organism using the metabolic pathway dynamics model.

In some embodiments, the first time-series multiomics data comprises time-series metabolomics data of a plurality of strains of an organism, wherein the time-series metabolomics data comprises two or more time-series of a strain. The second time-series multiomics data can comprise time-series proteomics data of a plurality of strains of an organism, and wherein the time-series proteomics data comprises a plurality of time-series of a strain. The first time-series multiomics data can comprise time-series multiomics data of a plurality of strains of an organism, and wherein the first time-series multiomics data comprises time-series multiomics data of a plurality of strains of a different organism.

The first time-series multiomics data or the second time-series multiomics data comprises time-series proteomics data, time-series metabolomics data, time-series transcriptomics data, or a combination thereof. The first time-series multiomics data or the second time-series multiomics data can be associated with an enzymatic characteristic selected from the group consisting of a k_{cat} constant, a Kₘ constant, and a kinetic characteristics curve. The first time-series multiomics data and the second time-series multiomics data can comprise observations at corresponding time points.

The machine learning model can comprise a supervised machine learning model. The machine learning model can comprises observable and unobservable parameters representing kinetics of the metabolic pathway.

Training the machine learning model can comprise training the machine learning model using training data comprising an n-tuples of a first observation at a time point in the first time-series multiomics data, a second observation at the time point in the second time-series multiomics data, and a derivative of the first observation. Training the machine learning model can comprise selecting the machine learning model from a plurality of machine learning models using a tree-based pipeline optimization tool.

Simulating the virtual strain of the organism can comprise integrating derivatives of the first time-series multiomics data outputted by the metabolic pathway dynamics model. Simulating a virtual strain of the organism using the metabolic pathway dynamics model can comprise simulating a virtual strain using the metabolic pathway dynamics model to change one or more of titer, rate, and yield of a product of a metabolic pathway represented by the metabolic pathway dynamics.

The method can comprise designing a strain of the organism corresponding to the simulated strain. The method can comprise creating a strain of the organism corresponding to the simulated strain.

### Overview

Increasingly computational biology is focusing on large scale modeling of dynamical systems as a way to better predict phenotype from genotype. Modeling of these complex systems has been made possible in part due to advances in high throughput data collection. For example, transcriptomics data volume has a doubling rate of seven months. The collection of large data sets has allowed for fitting of increasing complex parametric models. As models become more complex, fitting and troubleshooting these models can require more time from domain experts.

Disclosed herein are systems and methods for determining complex cellular dynamics, including non-linear dynamics, from observed data within the organism. The systems and methods can be used to approximate the dynamical behavior of these biological systems. In one example, the method can utilize non-linear identification methods. The model determined can be used for design and optimization of synthetic pathways. Some or all of the relevant dynamic quantities used to learn the models can be time series observations. The model learned can be used for predicting the dynamic behavior of a system from proteomics data specific to a metabolic subnetwork of interest. The methods disclosed herein can be scalable, resulting in enhanced predictive capacity.

### Data Driven Model Creation

Embodiments relate to systems and method for combining machine learning and multiomics data (such as proteomics and metabolomics data) to effectively predict pathway dynamics of a living organism in an automated manner. The system may not assume any particular interactions, but rather implicitly chooses or models the most predictive interactions.

### Biological Modeling of Large Metabolic Systems Involving Complex Dynamics

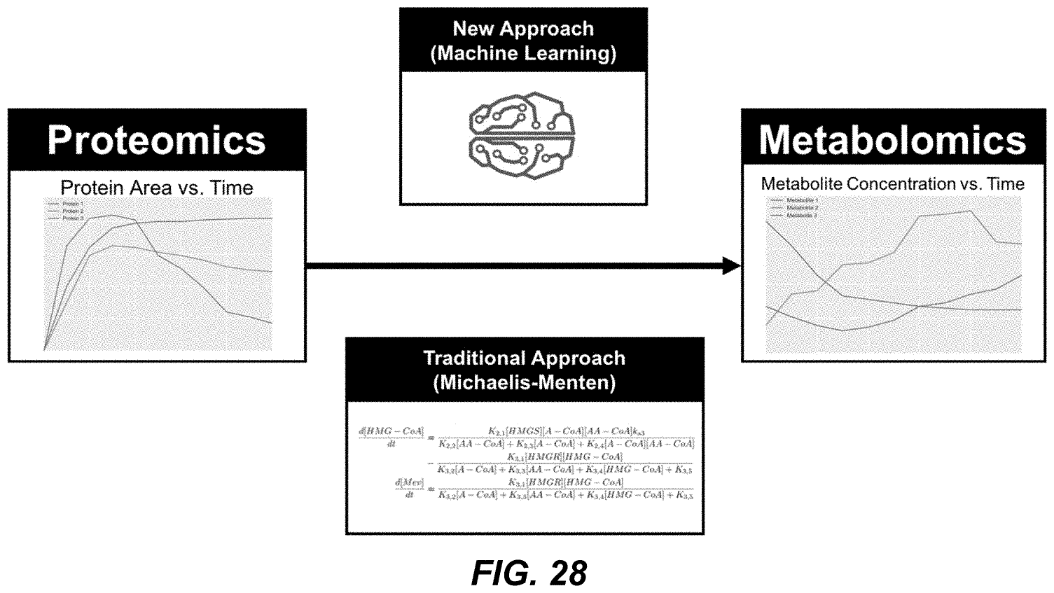

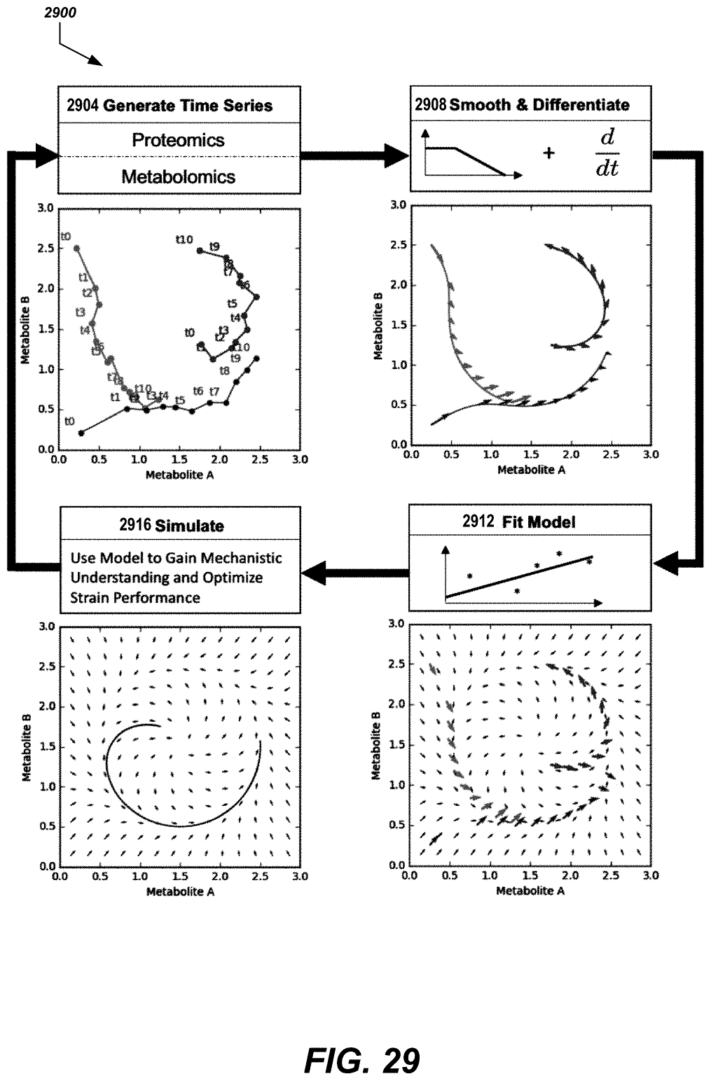

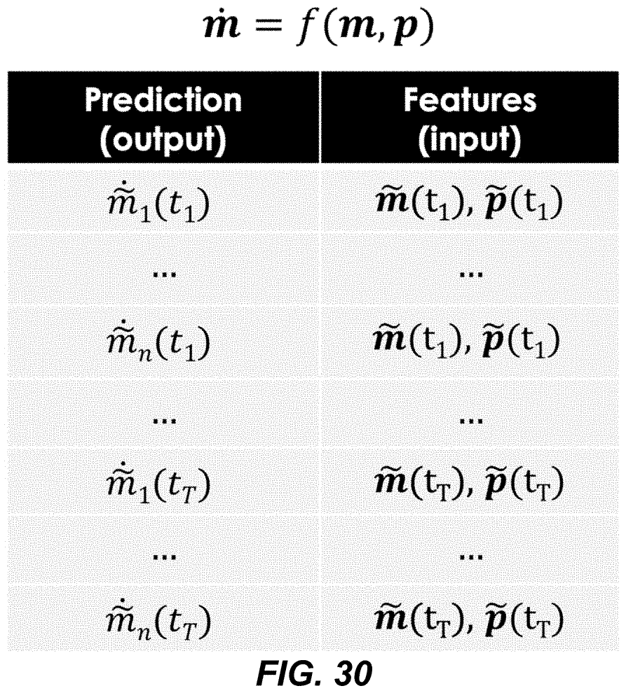

Disclosed herein are embodiments of a method for modeling metabolic pathway dynamics involving a machine learning (ML) approach (FIGS. 28 and 29). The function that determines the rate of change for each metabolite from protein and metabolite concentrations may be directly learned from training data (Eq. (1) and FIG. 30), without presuming any specific relationship.

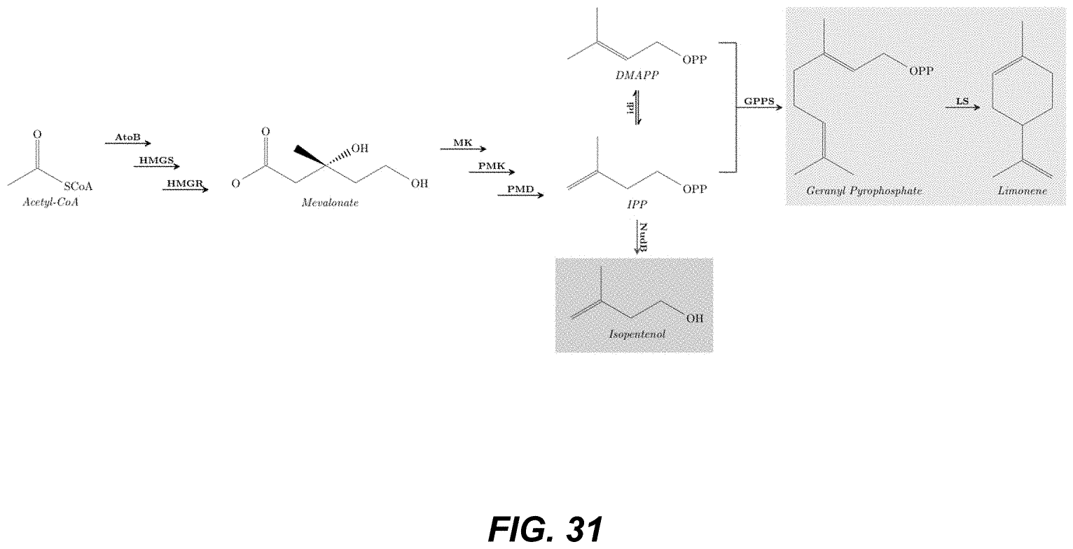

This machine learning-based approach may provide a faster development of predictive pathway dynamics models since all required knowledge (regulation, host effects, etc.) may be inferred from experimental data, instead of arduously gathered and introduced by domain experts (see below for an example). In this way, the method provides a general approach, valid even if the host is poorly understood and there is little information on the heterologous pathway, and provides a systematic way to increase prediction accuracy as more data is added. This method may obtain better predictions than the traditional Michaelis-Menten approach. For example, the ML-based method may generate better predictions than a model based on Michaelis-Menten kinetics for the limonene and isopentenol producing pathways studied here (FIG. 31) using only two times series, corresponding to data generated by two strains. The prediction performance of the ML-based model may improve as more time-series data is added. The new method was found to be accurate enough to drive bioengineering efforts to create modified strains. The disclosed methods are scalable to genome-scale models and/or generally applicable to other types of data (e.g., transcriptomics) or dynamic systems (e.g., microbiome dynamics).

Disclosed herein are methods that use protein levels of an organism to predict times series of metabolite concentrations. Understanding this type of pathway dynamics allows an accurate prediction of the behavior of the pathway. This also may allow the reliable design of specific biological systems, such as strains bioengineered to produce particular chemical products. Embodiments may automatically learn these pathway dynamics from previously obtained metabolomics and proteomics data using machine learning approaches. For example, the method may include receiving sets of proteomics and metabolomics data collected for several strains of one or more organisms of different species and then applying a supervised learning process to the time-series data and its derivatives to predict metabolite time-series data from the proteomics data. This model can then be tested for new strains with improved predictive ability.

### Supervised Learning of Metabolic Pathway Dynamics

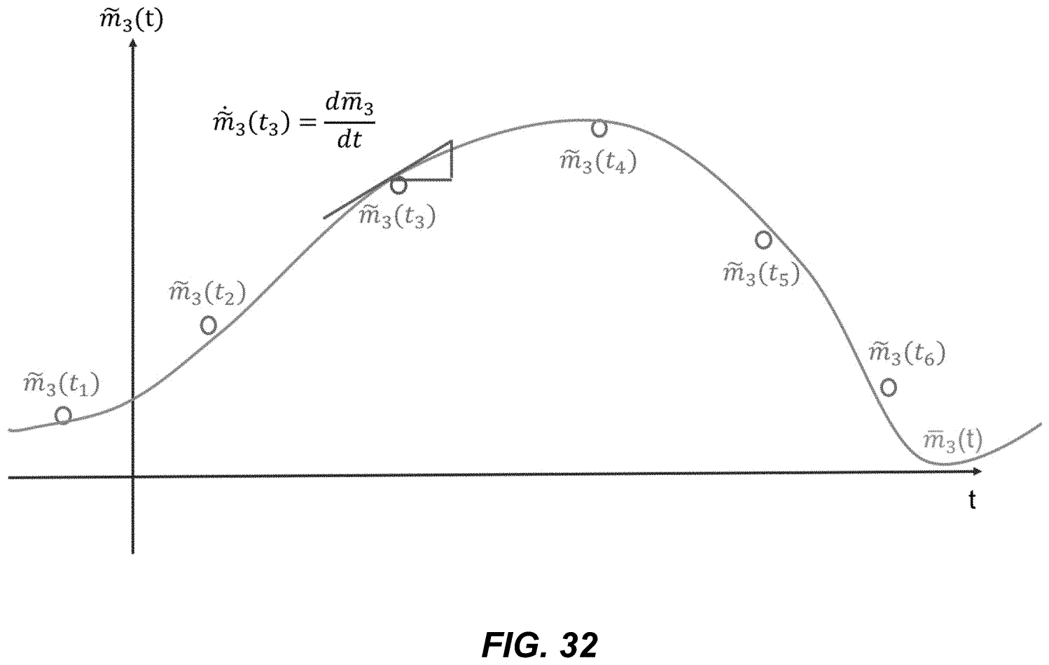

Assume there are q sets of time series metabolite \tilde{m}ⁱ[t]∈ [symbol] ⁿ (FIG. 32) and protein \tilde{p}ⁱ[t]∈ [symbol] observations at times T=[t₁, t₂, . . . tₛ]∈ [symbol] ₊^{s}. The superscript i∈{1, . . . , q} indicates the time-series index (strain), and \tilde{m}[t]=[\tilde{m}₁[t], . . . , \tilde{m}ₙ[t]]^{T} and \tilde{p}[t]=[\tilde{p}₁[t], . . . , \tilde{p}ₙ[t]]^{T} are vectors of measurements at time t containing concentrations for the n metabolites and l proteins considered in the model. The number of observation time points should be dense enough to capture the dynamic behavior of the system.

Assume that the underlying continuous dynamics of the system, which generates these time-series observations, can be described by coupled nonlinear ordinary differential equations of the general type used for kinetic modeling:

\dot{m}=ƒ(m(t),p(t)) (1)

where m and p are vectors that denote the metabolite and protein concentrations. The function ƒ: [symbol] ⁿ^{^{+l}}→ [symbol] ⁿ encloses all the information on the system dynamics. Deriving these dynamics from the time-series data can be formulated as a supervised learning problem where the function ƒ is learned through machine learning methods, which predict the relationship between metabolomics and proteomics concentrations (input features, see FIG. 30) and the metabolite time derivative IMO (output). In order to provide the training data set for this problem, the metabolite time derivative can obtained from the times-series data \tilde{m}(t), as shown in FIG. 32.

In order to parametrize the machine learning process, the following optimization problem can be solved (such as through scikit-learn):

Supervised Learning of Metabolic Dynamics. Find a function ƒ which satisfies:

$$arg {min}_{f} {\sum }_{i = 1}^{q} {\sum }_{t ∈ T} {‖ f ( {\tilde{m}}^{i} [ t ] , {\tilde{p}}^{i} [ t ] ) - {\dot{\tilde{m}}}^{i} ( t ) ‖}^{2} . \qquad ( 2 )$$

Finding the function ƒ can be considered equivalent to finding the metabolic dynamics, which describe the time-series data provided. Once the dynamics are learned, the behavior of the metabolic pathway can be predicted by solving an initial value problem (Eqs. (3) and (4)).

Learning System Dynamics from Time-Series Data

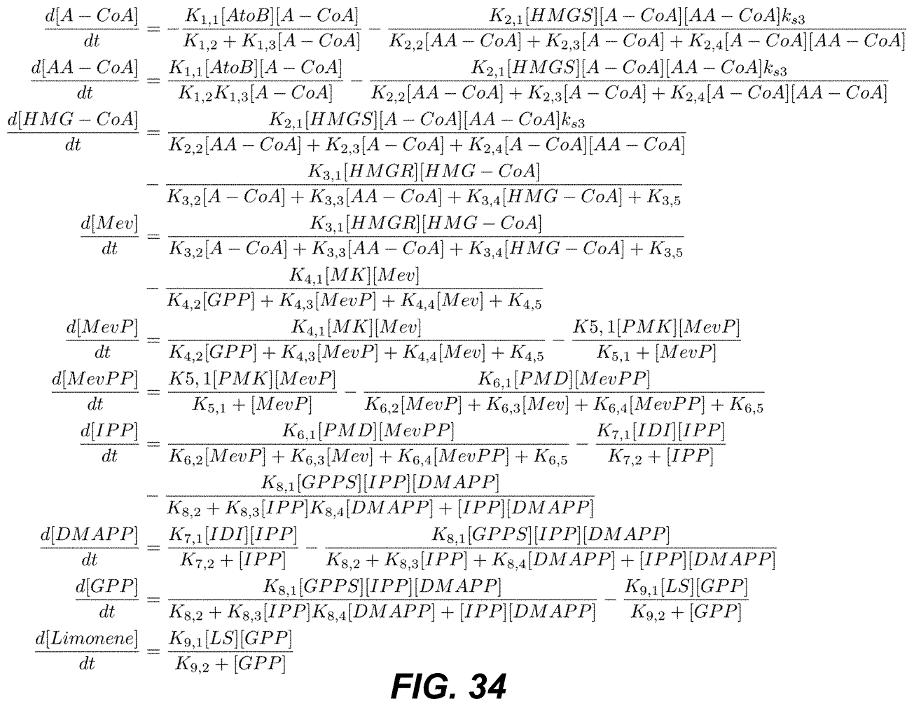

The methods for determining dynamics of metabolic pathways disclosed herein can include using machine learning methods to predict the functional relationship between the metabolite derivative and proteomics and metabolomics data. The methods can include substituting the Michaelis-Menten relationship (Eq. (1), FIG. 30 and FIG. 34). The first step can involve creating a training set comprising sets of proteomics and metabolomics data and their corresponding derivatives (FIG. 30). This can include computing the derivatives of the metabolite concentration time-series data. Because the time-series data may be subject to measurement noise, in some embodiments the derivatives must be carefully estimated. The second step involves finding the best performing regression technique, among the many possibilities available. Finally, once the best performing regression technique is found and cross-validated, it can be used to predict metabolite concentrations given initial time points. The complete code to implement these steps is provided in github.

Construction of the Training Data Set

In order to train a machine learning model, a suitable training set has to be created. The trained machine learning model may take in metabolite and protein concentrations at a particular point in time and return the derivative of the metabolite concentrations at the same time point (FIG. 30). The observations provide the inputs to the model, \tilde{m}ⁱ[t] and \tilde{p}[t]. In order to have examples of correct outputs for supervised learning, the derivatives of the metabolite time-series data, \dot{\tilde{m}}ⁱ(t), can be estimated (FIG. 32).

Naively computing the derivative of a noisy signal may amplify the noise and make the result unusable. Derivatives of noisy signals, like those obtained from experiments, may require extra effort to estimate. In order to estimate the time derivatives on time series of real data obtained from Brunk et al. (Characterizing strain variation in engineered E. coli using a multiomics-based workflow. Cell Syst. 2, 335-346 (2016); the content of which is incorporated herein by reference in its entirety. Data is available at the code repository: github.com/JBEI/KineticLearning) accurately, a Savitzky-Golay filter (Smoothing and differentiation of data by simplified least squares procedures. Anal. Chem. 36, 1627-1639 (1964); the content of which is incorporated herein in its entirety) was applied to the noisy time-series data to find a smooth estimate of the data (FIG. 32). This smooth function estimate can then be used to compute a more accurate estimate of the derivative. The derivative estimate of the signal can be computed using a central difference scheme from the filtered experimental data. Specifically, the Savitzky-Golay filter can be used with a filter window of 7 and a polynomial order of 2. The derivative estimate, \dot{\tilde{m}}ⁱ(t) can be computed for all time points in T and time series i. This results in a training example associated with each time point in every time series.

In one implementation, all relevant metabolites are measured and the system may be assumed to have no unmeasured memory states. In other words, the present set of metabolite and protein measurements completely determines the metabolite derivatives at the next time instant. If this assumption does not hold practically, a limited time history of proteins and metabolites can be used to predict the derivative at the next time instant. This assumption produces good predictions for some metabolic pathways, such as those described herein.

Model Selection

In one implementation, the model selection process can be implemented using a meta-learning package in python called Tree-based Pipeline Optimization Tool (TPOT; available at epistasislab.github.io/tpot/). Once the training data set is established, a machine learning model can be selected to learn the relationship between input and outputs (FIG. 30). TPOT uses genetic processes to find a model with the best cross-validated performance on the training set. Cross validation techniques may be used to score an initial set of models. The best performing models may be mated to form a new population of models to test. This process may be repeated for a fixed number of generations and the best performing model may be returned to the user. If desired, the search space for model selection can be specified before execution of the TPOT regressor search. This might be done to prune models that require long training times or to select only models that have desirable properties for the problem under consideration. Specifically, TPOT may be used to select the best pipelines it can find from the scikit-learn library combining 11 different regressors and 18 different preprocessing processes. This model selection process can done independently for each metabolite (Table 1). After TPOT determines the optimal models associated with each metabolite, the models are trained on the data set of interest and are ready for use to solve Eqs. (3) and (4). Models with the lowest tenfold cross-validated prediction root mean squared error may be selected. In this way, the best validated models are selected for use.

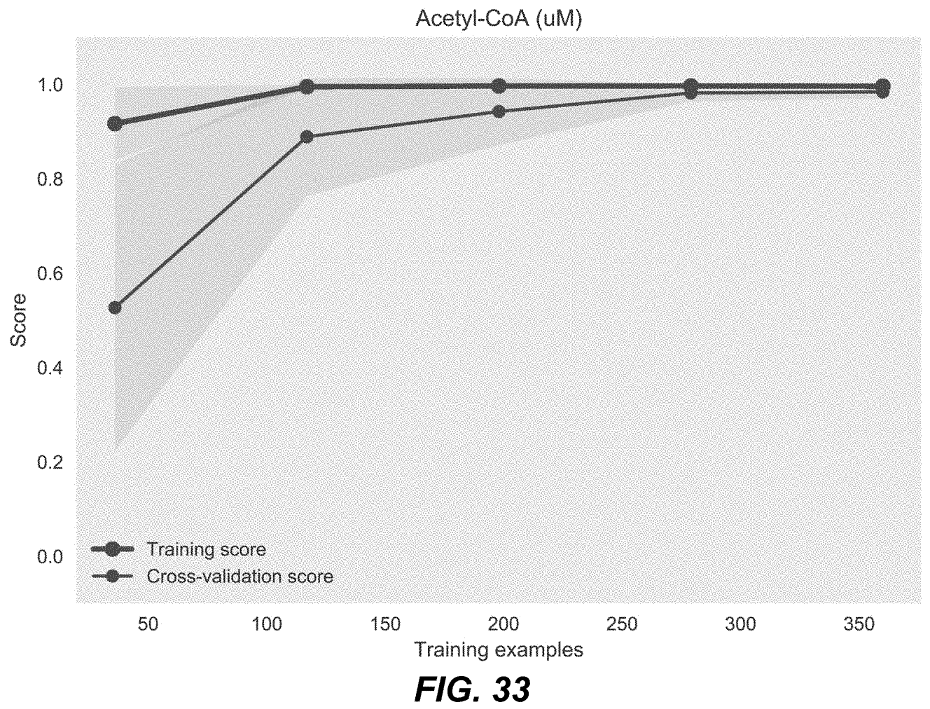

After automated model selection via TPOT, each model may be evaluated based on its accuracy in predicting metabolite derivatives given protein and metabolite concentration at a given time point (FIG. 30). Each data set used for model fitting can be split into training and test sets ten times using the shuffle split methodology implemented in scikit-learn. After the model is fit, predictions on both the training and test sets may be computed for each metabolite model and their predictive ability quantified through a Pearson R² coefficient (e.g., FIG. 33).

Using the model. Once the models are trained, they can be used to predict metabolite concentrations by solving the following initial value problem using the same function ƒ learned in Eqs. (1) and (2):

\dot{m}=ƒ(m,\tilde{p}) (3)

m(t₀)=\tilde{m}(t₀) (4)

This problem can be solved by integrating the system forward in time numerically. As a general purpose numerical integrator, a Runga Kutta 45 implementation may be used.

### Data Set Curation and Synthesis

A number of different data sets may be used. The first may be an experimental data set curated from a previous publication, comprising three proteomic and metabolomic time-series (strains) from an isopentenol producing E. coli and three time-series (strains) from limonene producing E. coli. The second data set may involve computationally simulated data from a kinetic model of the limonene pathway, which may be used to test how the method performance scales with the number of time series used.

Description of a real time-series multiomics data set. Proteomics and metabolomics data for two different heterologous pathways engineered into an organism, such as the bacterium E. coli, may be obtained. There may be three (high, medium, and low production) variants for strains which produce isopentenol and limonene, respectively. All strains may be derived from E. coli DH1. The low and high-producing strain for each pathway may be used to predict the medium production strain dynamics by solving Eqs. (3) and (4).

The isopentenol producing strains (I1, I2 and I3) may be engineered to contain all of the proteins required to produce isopentenol from acetyl-CoA as (FIG. 31). I1 may be the unoptimized strain containing the naive variants of each protein in the pathway. I2 may differ from the base strain I1 in that it contained a codon optimized HMGR enzyme along with the positions of PMK and MK swapped on its operon. I3 may use a homolog, such as an HMGR homolog from Staphylococcus aureus.

Limonene producing strains (L1, L2, and L3) may produce limonene from acetyl-CoA (FIG. 31). L1 may be the un-optimized strain with the naively chosen variants for each protein in the pathway. It may have a two plasmid system where the lower and upper parts of the pathway are split between both constructs. L2 may be a DH1 variant that contains the entire limonene pathway on a single plasmid. L3 may be another two-plasmid strain where the entire pathway is present on the first plasmid, and the terpene synthases are on a second plasmid for increased expression. Starting at induction, each strain may have measurements taken at seven time points during fermentation over 72 hours. At each time point pathway, metabolite measurements and pathway protein measurements may be collected.

Data augmentation through filtering and interpolation. In the training set each time series may contain a number of data points, such as seven data points. These may be too sparse to formulate accurate models. To overcome this a data augmentation scheme may be employed where seven time points from the original data are expanded into 200 for each strain. This may be done by smoothing the data with a Savitzky-Golay filter and interpolating over the filtered curve (FIG. 29 and FIG. 32). When predicting the dynamics of a medium production strain from high and low producing strains, model selection may be performed by scoring each model using tenfold cross validation and a Pearson R² metric on two data augmented training strains.

Development of realistic kinetic models. To study the scaling of performance as more training sets are added, a realistic and dynamically complex model of the mevalonate pathway may be developed from known interactions extracted from the literature (FIGS. 31 and 34). The dynamic model may be implemented with Michaelis-Menten like kinetics and may be a 10-state coupled nonlinear system. Exemplary details of this kinetic model are described below. The objective may be to create a realistic model, relevant to metabolic engineering, for which learning the system dynamics is a non-trivial task on par with the difficulty of learning real system dynamics from experimental data.

Generation of a simulated data set. The kinetic model described above may be used to create a set of virtual data time-series (strains). The kinetic model coefficients may be chosen to be close to values available, such as values reported in the literature, while maintaining a non-trivial dynamic behavior.

A virtual strain may be created by first generating a pathway proteomic time series. This may be done by randomly choosing three coefficients for each protein (k_{ƒ}, kₘ, kₗ), which specify a leaky hill function. The hill function may be used because it models the dynamics of protein expression from RNA accurately. This leaky hill function specifies the protein measurements for each time point and is defined in the eq. (5) below:

$$\tilde{p} ( t ) = \frac{{k}_{f} t}{{k}_{m} + t} + {k}_{l} \qquad ( 5 )$$

Once all protein time series are specified, they may be used in conjunction with the kinetic coefficients to solve the initial value problem in Eqs. (3) and (4) in order to determine the time series of metabolite concentrations. The resulting data set may be a collection of time-series measurements of different strain proteomics and metabolomics. All or some strains may use the same kinetic parameters and differential equations to generate the metabolomics measurements.

### Fitting the Michaelis-Menten Kinetic Model

To compare the handcrafted kinetic model with the data-centric machine learning methodology, the parameters of the kinetic model may be fitted to strain data. To find the best fit, a differential evolution algorithm or process implemented in scipy may be used. This global optimizer may be chosen because its convergence is independent of the initial population choice and it tends to need less parameter tuning than other methods. All kinetic parameters may be constrained to be between 10⁻¹² and 10⁹, for example. This large range of acceptable parameter values may allow for maximum flexibility of the kinetic model to describe the data.

### Evaluation of Model Performance for Time Series

Dynamical prediction may be tested on a held back strain that is not used to train the model. When using the experimental data sets, the medium titer strains may be held back for testing. When using simulated data, a random strain from the data set may be selected. For each time series, agreement between predictions and test data may be assessed by calculating the root mean squared error (RMSE) of the predicted trajectories:

$$R M S E = \sqrt{\frac{1}{n} {\sum }_{j = 1}^{n} {\int }_{{t}_{0}}^{{t}_{f}} {( \overline{{m}_{j}} ( t ) - {m}_{j} ( t ) )}^{2} dt} , \qquad ( 6 )$$

where m̅ⱼ(t) is the interpolation of the actual metabolite concentration of metabolite j at time t (FIG. 32), and mⱼ(t) is the prediction obtained from solving Eqs. (3) and (4).

### Example Learning Process and Strain Creation

Many machine learning techniques can be used to solve supervised learning problems. The techniques may use computational models trained to predict dependent variables from independent variables. A real valued dependent variable vector of protein and metabolite concentrations at a particular time point can be related to the derivatives of metabolite concentrations at the same time point. Learning these derivatives at a particular system state of a biological system can be equivalent to learning the dynamics of the entire biological system. Learning these derivatives can be possible because the independent variables contained sufficient information to predict dependent variables.

FIG. 29 shows a schematic illustration of a process 2900 for learning metabolic pathway dynamics from time series proteomics and metabolomics data (or multiomics data general). In a cyclic fashion, cellular dynamics can be learned and used for mechanistic understanding or metabolic engineering. At block 2904, time series experimental data (e.g., proteomics and metabolomics data) can be generated or acquired for several strains of an organism of interest.

At block 2908, the time-series data traces can be smoothed and differentiated. Because the time-series data can be subject to measurement noise, estimating the derivatives carefully can be important. For example, a filter (e.g., a Savitzky-Golay filter) can be first applied to the noisy time-series data to find a smooth estimate of the data. This smooth function estimate can then be used to compute a more accurate estimate of the derivative. Once both the independent and dependent variable pairs have been created for training, a machine learning process can be applied to find the vector field which describes the metabolic system dynamics. The machine learning method can be a regressor, such as a random forest regressor. The regressor can be a metabolic engineering-specific, supervised learning regressor that restricts the function search space to the set of possible kinetic models. The derivatives help to provide examples of the dynamics at the states explored by each strain.

At block 2912, the state-derivative pairs can be fed into a supervised learning method, such as a random forest regression method, to determine a metabolic pathway dynamic model representing the metabolic system dynamics of the organism. In one embodiment, the state can be represented by a protein concentration and a metabolite concentration. The machine learning method can be used to learn and generalize the metabolic system dynamics from the state-derivative pairs of each strain. For example, the data can be used to learn the relationships between each state and the corresponding derivative. Each unique strain can be modeled to have a unique proteomics profile, and the time-series proteomics data can be unique for each strain. At block 2916, the model can then be used to simulate virtual strains and explore the metabolic space looking for mechanistic insight or commercially valuable designs. This process can then be repeated using the model to create new strains, which can further improve the accuracy of the dynamic model.

Each pathway dynamic model used to create simulated training data included free parameters which represent pathway kinetics, and exogenous variables which allow virtual strains to be expressed. Each unique strain was modeled to have a unique proteomics profile, and the time-series proteomics data was unique for each strain. When generating data, a realistic set of kinetic parameters for the pathway was randomly generated. Then a time-series data set corresponding to each virtual strain was generated. For training purposes, as many as 10,000 strains were generated at a time. As a result the data set was a collection of time-series of different strain proteomics and metabolomics data for a pathway with shared kinetic parameters.

The models learned can be useful for metabolic engineering. Having a predictive model of the dynamics of a metabolic network can allow rational engineering of strains for various objectives. Metabolic engineering can include maximizing titer or yield of a valuable biochemical. A dynamical model can be queried for strains which improve on existing design goals. In one embodiment, the method 200 can include designing a strain of the organism that corresponds to one of the strains simulated. The method 200 can include creating a strain of the organism corresponding to the simulated strain. The simulated strain can have one or more desired characteristics of the strain, such as titer, rater, and yield of a product of the metabolic pathway represented the metabolic pathway dynamic model. The method 200 may include receiving time-series proteomics and metabolomics data of the created strain. The model may be retrained using the time-series proteomics and metabolomics data of the created strain.

In one embodiment, a method 200 for simulating the metabolic pathway dynamics of a strain of an organism comprises: receiving time-series multiomics data comprising a first time-series multiomics data associated a metabolic pathway and a second time-series multiomics data associated with the metabolic pathway at block 2904; determining derivatives of the first time-series multiomics data at block 2908; training a machine learning model, representing a metabolic pathway dynamics model, using the first time-series multiomics data, the derivatives of the first time-series multiomics data, and the second time-series multiomics data, wherein the metabolic pathway dynamics model relates the first time-series multiomics data and the second time-series multiomics data to the derivatives of the first time-series multiomics data at block 2912; and simulating a virtual strain of the organism using the metabolic pathway dynamics model at block 2916. The method 200 may include designing a strain of the organism corresponding to the simulated strain, and/or creating a strain of the organism corresponding to the simulated strain.

The first time-series multiomics data may include time-series metabolomics data of a plurality of strains of an organism, and the time-series metabolomics data may include two or more time-series of a strain. The second time-series multiomics data may include time-series proteomics data of a plurality of strains of an organism, and the time-series proteomics data may include a plurality of time-series of a strain. The first time-series multiomics data may be, or include, time-series multiomics data of a plurality of strains of an organism, and wherein the first time-series multiomics data comprises time-series multiomics data of a plurality of strains of a different organism. The first time-series multiomics data or the second time-series multiomics data may be, or include, time-series proteomics data, time-series metabolomics data, time-series transcriptomics data, or a combination thereof. The first time-series multiomics data or the second time-series multiomics data may be associated with an enzymatic characteristic selected from the group consisting of a k_{cat} constant, a Kₘ constant, and a kinetic characteristics curve. The first time-series multiomics data and the second time-series multiomics data may include observations at corresponding time points.

The machine learning model may include a supervised machine learning model. The metabolic pathway dynamics model may include observable and unobservable parameters representing kinetics of the metabolic pathway. Training the machine learning model may include training the machine learning model using training data comprising an n-tuples of a first observation at a time point in the first time-series multiomics data, a second observation at the time point in the second time-series multiomics data, and a derivative of the first observation. Training the machine learning model may include selecting the machine learning model from a plurality of machine learning models using a tree-based pipeline optimization tool. Simulating the virtual strain of the organism may include integrating derivatives of the first time-series multiomics data outputted by the metabolic pathway dynamics model. Simulating a virtual strain of the organism using the metabolic pathway dynamics model may include simulating a virtual strain using the metabolic pathway dynamics model to change one or more of titer, rate, and yield of a product of a metabolic pathway represented by the metabolic pathway dynamics.

### Development of a Kinetic Model for Limonene Synthesis

Below is an exemplary description of each reaction in the limonene pathway including likely inhibiting metabolites. The descriptions provide a solid starting point for a mechanistic metabolic model for limonene production.

Reaction 1

Acetyl-CoA is converted to acetoacetyl-CoA using acetyl-CoA acetyltransferase (AtoB) using a ping-pong mechanism. This enzyme is inhibited by:

$$2 acetyl - CoA {\to }^{AtoB} CoA + acetoacetyl - CoA .$$

The ping pong mechanism of this reaction is illustrated as:

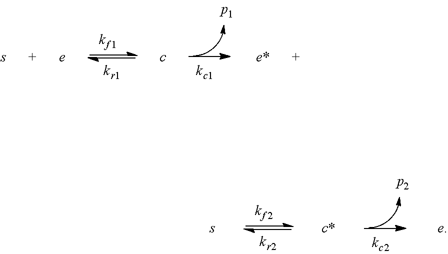

The mass action law describing this mechanism of reaction 1 (R1) may be described by the following system of ordinary differential equations.

$${R}_{1} \{ \begin{matrix}\dot{s} = {k}_{r 1} c + {k}_{r 2} {c}^{*} - {k}_{f 1} se - {k}_{f 2} {se}^{*} \\ \dot{e} = {k}_{r 1} c + {k}_{c 2} {c}^{*} - {k}_{f 1} se \\ \dot{c} = {k}_{f 1} se - {k}_{r 1} c - {k}_{c 1} c \\ {\dot{p}}_{1} = {k}_{c 1} c \\ {\dot{e}}^{*} = {k}_{c 1} c + {k}_{r 2} {c}^{*} - {k}_{f 2} {se}^{*} \\ {\dot{c}}^{*} = {k}_{f 2} {se}^{*} - {k}_{r 2} {c}^{*} - {k}_{c 2} {c}^{*} \\ {\dot{p}}_{2} = {k}_{c 2} {c}^{*}\end{matrix}$$

Using the quasi-steady state assumption this can be rewritten in a Michaelis-Menten formulation. The resulting equation which describes the pathway product in terms of substrate concentrations is given by:

$${\dot{p}}_{2} = \frac{{K}_{1} {e}_{0} s}{{K}_{2} + {K}_{3} s}$$

$${\dot{p}}_{2} = \frac{{K}_{1} {e}_{0} s}{{K}_{2} + {K}_{3} s} ,$$

where

K₁=k_{c1}k_{c2}k_{ƒ1}k_{ƒ2}

K₂=k_{c1}k_{c2}k_{ƒ2}+k_{c1}k_{ƒ1}(k_{c2}+kᵣ₂)+k_{c2}k_{ƒ2}kᵣ₁

K₃=(k_{c1}+k_{c2})k_{ƒ1}k_{ƒ2}

Reaction 2

Acetoacetyl-CoA is converted to HMG-CoA by HMGS using a three-step ping pong mechanism reaction involving an acylation, a condensation, and a hydrolysis. The reaction is given by:

$$acetyl - CoA + acetoacetyl - CoA + {H}_{2} O {\to }^{HMG 8} HMG - CoA + CoA .$$

The three step ping pong mechanism is as shown below:

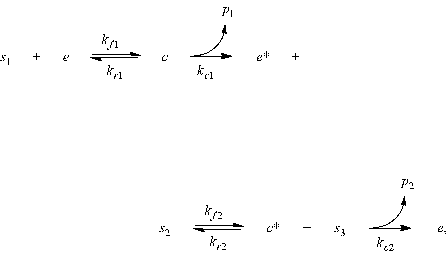

where p₁ is CoA and p₂ is HMG-CoA. The resulting differential equations for this system are given by:

$${R}_{2} \{ \begin{matrix}{\dot{s}}_{1} = {k}_{r 1} c - {k}_{f 1} {s}_{1} e \\ \dot{e} = {k}_{r 1} c + {k}_{c 2} {s}_{3} {c}^{*} - {k}_{f 1} {s}_{1} e \\ \dot{c} = {k}_{f 1} {s}_{1} e - {k}_{r 1} c - {k}_{c 1} c \\ {\dot{p}}_{1} = {k}_{c 1} c \\ {\dot{e}}^{*} = {k}_{c 1} c + {k}_{r 2} {c}^{*} - {k}_{f 2} {s}_{2} {e}^{*} \\ {\dot{s}}_{2} = {k}_{r 2} {c}^{*} - {k}_{f 2} {s}_{2} {e}^{*} \\ {\dot{c}}^{*} = {k}_{f 2} {s}_{2} {e}^{*} - {k}_{c 2} {s}_{3} {c}^{*} - {k}_{r 2} {c}^{*} \\ {\dot{s}}_{3} = - {k}_{c 2} {s}_{3} {c}^{*} \\ {\dot{p}}_{2} = {k}_{c 2} {c}^{*}\end{matrix}$$

Assuming quasi-steady state and constant H₂O concentration yields the Michaelis-Menten Equations:

$${\dot{s}}_{1} = - \frac{{K}_{1} {e}_{0} {s}_{1} {s}_{2} {s}_{3}}{{K}_{2} {s}_{2} + {K}_{3} {s}_{1} + {K}_{4} {s}_{1} {s}_{2}}$$

$${\dot{s}}_{2} = - \frac{{K}_{1} {e}_{0} {s}_{1} {s}_{2} {s}_{3}}{{K}_{2} {s}_{2} + {K}_{3} {s}_{1} + {K}_{4} {s}_{1} {s}_{2}}$$

$${\dot{p}}_{2} = \frac{{K}_{1} {e}_{0} {s}_{1} {s}_{2}}{{K}_{2} {s}_{1} + {K}_{3} {s}_{2} + {K}_{4} {s}_{1} {s}_{2}} ,$$

where

K₁=k_{c1}k_{c2}k_{ƒ1}k_{ƒ2}

K₂=k_{c1}k_{c2}k_{ƒ2}s₃+k_{c2}k_{ƒ2}kᵣ₁s₃

K₃=k_{c1}k_{c2}k_{ƒ1}s₃+k_{c1}k_{ƒ1}kᵣ₂

K₄=k_{c1}k_{ƒ1}k_{ƒ2}+k_{c2}k_{ƒ1}k_{ƒ2}s₃

Reaction 3

Guessing an ordered sequential reaction mechanism with two competitive inhibitors with respect to HMG-CoA. This reaction is inhibited by acetyl-CoA and acetoacetyl-CoA. Because of similarity in substrate and inhibitor structure, it can assumed to be competitive with respect to HMG-CoA.

$$HMG - CoA + NADPH {\to }^{HMGB} Mevalonate + NAPD + {s}_{1} + e {{⇌}_{{k}_{r 1}}}^{{k}_{f 1}} {c}_{1} + {s}_{2} {{⇌}_{{k}_{r 2}}}^{{k}_{f 2}} {c}_{2} {\to }^{{k}_{c 1}} {c}_{3} {{⇌}_{{k}_{r 3}}}^{{k}_{f 3}} {c}_{4} + {p}_{1} {{⇌}_{{k}_{r 4}}}^{{k}_{f 4}} e + {p}_{2}$$

$${R}_{3} \{ \begin{matrix}{\dot{s}}_{1} = {k}_{r 1} {c}_{1} - {k}_{f 1} {s}_{1} e \\ \dot{e} = {k}_{r 1} {c}_{1} - {k}_{f 1} {s}_{1} e + {k}_{f 4} {c}_{4} - {k}_{r 4} {p}_{2} e - {k}_{fi 1} {ei}_{1} - {k}_{fi 2} {ei}_{2} \\ {\dot{c}}_{1} = {k}_{f 1} {s}_{1} e - {k}_{r 1} {c}_{1} - {k}_{f 2} {s}_{2} {c}_{1} + {k}_{r 2} {c}_{2} \\ {\dot{s}}_{2} = {k}_{r 2} {c}_{2} - {k}_{f 2} {s}_{2} {c}_{1} \\ {\dot{c}}_{2} = {k}_{f 2} {s}_{2} {c}_{1} - {k}_{r 2} {c}_{2} - {k}_{c 1} {c}_{2} \\ {\dot{c}}_{3} = {k}_{c 1} {c}_{2} + {k}_{r 3} {p}_{1} {c}_{4} - {k}_{f 3} {c}_{3} \\ {\dot{c}}_{4} = {k}_{f 3} {c}_{3} - {k}_{r 3} {p}_{1} {c}_{4} + {k}_{r 4} {p}_{2} e - {k}_{f 4} {c}_{4} \\ {\dot{c}}_{5} = {k}_{fi 1} {ei}_{1} - {k}_{ri 1} {c}_{5} \\ {\dot{c}}_{6} = {k}_{fi 2} {ei}_{2} - {k}_{ri 2} {c}_{6} \\ {\dot{p}}_{1} = {k}_{f 3} {c}_{3} - {k}_{r 3} {p}_{1} {c}_{4} \\ {\dot{p}}_{2} = {k}_{f 4} {c}_{4} - {k}_{r 4} {p}_{2} e \\ {\dot{i}}_{1} = - {k}_{fi 1} {ei}_{1} + {k}_{ri 1} {c}_{5} \\ {\dot{i}}_{2} = - {k}_{fi 2} {ei}_{2} + {k}_{ri 2} {c}_{5}\end{matrix}$$

Assuming a roughly constant ratio of NADPH to NADP+ and quasi-steady state enzyme balance we can write these equations more simply as:

$${\dot{s}}_{1} = - \frac{{K}_{1} {e}_{0} s}{{K}_{2} {i}_{1} + {K}_{3} {i}_{2} + {K}_{4} s + {K}_{5}}$$

$${\dot{p}}_{1} = \frac{{K}_{1} {e}_{0} s}{{K}_{2} {i}_{1} + {K}_{3} {i}_{2} + {K}_{4} s + {K}_{5}} .$$

Reaction 4

Mevalonate kinase (MK) proceeds via an ordered sequential mechanism, where mevalonate binds to the enzyme first, followed by ATP. After catalysis, phosphomevalonate is released followed by ADP:

$$ATP + mevalonte {\to }^{MK} ADP + phosphomevalonte .$$

The ordered sequential mechanism for Mevalonate Kinase:

$${s}_{1} + e {{⇌}_{{k}_{r 1}}}^{{k}_{f 1}} {c}_{1} + {s}_{2} {{⇌}_{{k}_{r 2}}}^{{k}_{f 2}} {c}_{2} {\to }^{{k}_{c 1}} {c}_{3} {{⇌}_{{k}_{r 3}}}^{{k}_{f 3}} {c}_{4} + {p}_{1} {{⇌}_{{k}_{r 4}}}^{{k}_{f 4}} e + {p}_{2}$$

$${R}_{4} \{ \begin{matrix}{\dot{s}}_{1} = {k}_{r 1} {c}_{1} - {k}_{f 1} {s}_{1} e \\ \dot{e} = {k}_{r 1} {c}_{1} - {k}_{f 1} {s}_{1} e + {k}_{f 4} {c}_{4} - {k}_{r 4} {p}_{2} e \\ {\dot{c}}_{1} = {k}_{f 1} {s}_{1} e - {k}_{r 1} {c}_{1} - {k}_{f 2} {s}_{2} {c}_{1} + {k}_{r 2} {c}_{2} \\ {\dot{s}}_{2} = {k}_{r 2} {c}_{2} - {k}_{f 2} {s}_{2} {c}_{1} \\ {\dot{c}}_{2} = {k}_{f 2} {s}_{2} {c}_{1} - {k}_{r 2} {c}_{2} - {k}_{c 1} {c}_{2} \\ {\dot{c}}_{3} = {k}_{c 1} {c}_{2} + {k}_{r 3} {p}_{1} {c}_{4} - {k}_{f 3} {c}_{3} \\ {\dot{c}}_{4} = {k}_{f 3} {c}_{3} - {k}_{r 3} {p}_{1} {c}_{4} + {k}_{r 4} {p}_{2} e - {k}_{f 4} {c}_{4} \\ {\dot{c}}_{5} = {k}_{fi 1} {ei}_{1} - {k}_{ri 1} {c}_{5} \\ {\dot{c}}_{6} = {k}_{fi 2} {ei}_{2} - {k}_{ri 2} {c}_{6} \\ {\dot{p}}_{1} = {k}_{f 3} {c}_{3} - {k}_{r 3} {p}_{1} {c}_{4} \\ {\dot{p}}_{2} = {k}_{f 4} {c}_{4} - {k}_{r 4} {p}_{2} e \\ {\dot{i}}_{1} = - {k}_{fi 1} {ei}_{1} + {k}_{ri 1} {c}_{5} \\ {\dot{i}}_{2} = - {k}_{fi 2} {ei}_{2} + {k}_{ri 2} {c}_{6}\end{matrix}$$

GPP and FPP are both competitive inhibitors of MK with respect to ATP. In the Streptococcus pneumoniae homolog of mevalonate kinase, diphosphomevalonate (DPM) is an noncompetitive inhibitor with respect to both substrates. DPM binds at an allosteric site, and inhibition cannot be overcome by an increasing substrate concentration.

The resulting Michaelis-Menten Equations Assuming ATP and ADP are roughly constant and two inhibitors:

$${\dot{s}}_{1} = - \frac{{K}_{1} {e}_{0} s}{{K}_{2} {i}_{1} + {K}_{3} {i}_{2} + {K}_{4} s + {K}_{5}}$$

$${\dot{p}}_{1} = \frac{{K}_{1} {e}_{0} s}{{K}_{2} {i}_{1} + {K}_{3} {i}_{2} + {K}_{4} s + {K}_{5}} .$$

Reaction 5

Phosphomevalonate Kinase proceeds with a random sequential bi-bi mechanism in the S. Pneumoniae homolog. The enzyme is kinetically characterized for S. Cerevisiae, however, it may be superior to use the better characterized enzyme in S. Pneumoniae.

$$ATP + phosphomevalonte {\to }^{PMK} ADP + pyromevalonte$$

$${s}_{1} + e {{⇌}_{{k}_{r 1}}}^{{k}_{f 1}} {c}_{1} + {s}_{2} {{⇌}_{{k}_{r 2}}}^{{k}_{f 2}} {c}_{2} {\to }^{{k}_{r 1}} {c}_{3} {{⇌}_{{k}_{r 3}}}^{{k}_{f 3}} {c}_{4} + {p}_{1} {{⇌}_{{k}_{r 4}}}^{{k}_{f 4}} e + {p}_{2}$$

$${R}_{5} \{ \begin{matrix}{\dot{s}}_{1} = {k}_{r 1 a} {c}_{1 a} - {k}_{f 1 a} {s}_{1 a} e + {k}_{r 2 b} {c}_{2} - {k}_{f 2 b} {s}_{1 a} {c}_{1 b} \\ {\dot{s}}_{2} = {k}_{r 1 b} {c}_{1 b} - {k}_{f 1 b} {s}_{1 b} e + {k}_{r 2 a} {c}_{2} - {k}_{f 2 a} {s}_{2} {c}_{1 a} \\ \dot{e} = {k}_{r 1 a} {c}_{1 a} + {k}_{f 1 b} {c}_{1 b} - {k}_{f 1 b} {s}_{2} e - {k}_{f 1 a} {s}_{1} e - {k}_{f 4 a} {c}_{4 a} + {k}_{f 4 b} {c}_{4 b} + {k}_{r 1 a} {p}_{2} e - {k}_{r 4 b} {p}_{1} e \\ {\dot{c}}_{1 a} = {k}_{f 1 a} {s}_{1} e - {k}_{r 1 a} {c}_{1} a - {k}_{r 2 a} {c}_{2} - {k}_{f 2 a} {s}_{2} {c}_{1 a} \\ {\dot{c}}_{1 b} = {k}_{f 1 b} {s}_{2} e - {k}_{r 1 b} {c}_{1} b - {k}_{r 2 b} {c}_{2} - {k}_{f 2 b} {s}_{1} {c}_{1 b} \\ {\dot{c}}_{2} = {k}_{f 2 a} {s}_{2} {c}_{1 a} - {k}_{r 2 a} {c}_{2} + {k}_{f 2 b} {s}_{1} {c}_{1 b} - {k}_{r 2 b} {c}_{2} - {k}_{c} {c}_{2} \\ {\dot{c}}_{3} = {k}_{c} {c}_{2} + {k}_{r 3 a} {c}_{4 a} {p}_{1} - {k}_{f 3 a} {c}_{3} + {k}_{r 3 b} {c}_{4 b} {p}_{2} - {k}_{f 3 b} {c}_{3} \\ {\dot{p}}_{1} = {k}_{f 3 a} {c}_{3} - {k}_{r 3 a} {c}_{4 a} {p}_{1} + {k}_{f 4 b} {c}_{4 b} - {k}_{r 4 b} {p}_{1} e \\ {\dot{p}}_{2} = {k}_{f 3 b} {c}_{3} - {k}_{r 3 b} {c}_{4 b} {p}_{2} + {k}_{f 4 a} {c}_{4 a} - {k}_{r 4 a} {p}_{2} e \\ {\dot{c}}_{4 a} = {k}_{f 3 a} {c}_{3} - {k}_{r 3 a} {c}_{4 a} {p}_{1} + {k}_{r 4 a} {c}_{4 a} - {k}_{r 4 a} {p}_{2} e \\ {\dot{c}}_{4 b} = {k}_{f 3 b} {c}_{3} - {k}_{r 3 b} {c}_{4 b} {p}_{2} + {k}_{r 4 b} {c}_{4 b} - {k}_{r 4 b} {p}_{1} e\end{matrix}$$

### Briggs-Haldane Kinetics:

$$\dot{s} = - \frac{{K}_{cat} {e}_{0} s}{{K}_{d} + s}$$

$$\dot{p} = \frac{{K}_{cat} {e}_{0} s}{{K}_{d} + s} ,$$

$$where$$

$${K}_{cat} = {k}_{c 1}$$

$${K}_{d} = \frac{{k}_{c 1} + {k}_{r 1}}{{k}_{f 1}} .$$

Reaction 6

PMD proceeds with an ordered sequential reaction mechanism. Ordered sequential mechanism with mevalonate 5-diphosphate as the first substrate to bind to the enzyme.

$$diphosphomevalonate + ATP {\to }^{PMD} ADP + phosphate + isopentenyl diphosphate + {CO}_{2}$$

$${s}_{1} + e {{⇌}_{{k}_{r 1}}}^{{k}_{f 1}} {c}_{1} + {s}_{2} {{⇌}_{{k}_{r 2}}}^{{k}_{f 2}} {c}_{2} {\to }^{{k}_{r 1}} {c}_{3} {{⇌}_{{k}_{r 3}}}^{{k}_{f 3}} {c}_{4} + {p}_{1} {{⇌}_{{k}_{r 4}}}^{{k}_{f 4}} {c}_{5} + {p}_{2} {{⇌}_{{k}_{r 5}}}^{{k}_{f 5}} {c}_{6} + {p}_{3} {{⇌}_{{k}_{r 6}}}^{{k}_{f 6}} e + {p}_{4}$$

$${R}_{6} \{ \begin{matrix}{\dot{s}}_{1} = {k}_{r 1} {c}_{1} - {k}_{f 1} {s}_{1} e \\ \dot{e} = {k}_{r 1} {c}_{1} - {k}_{f 1} {s}_{1} e + {k}_{f 6} {c}_{6} - {k}_{r 6} {p}_{4} e + {k}_{ri 1 a} {c}_{i 1 a} - {k}_{fi 1 a} {i}_{1} e + {k}_{ri 1 b} {c}_{i 1 b} - {k}_{fi 1 b} {i}_{2} e \\ {\dot{c}}_{1} = {k}_{f 1} {s}_{1} e - {k}_{r 1} {c}_{1} - {k}_{f 2} {s}_{2} {c}_{1} + {k}_{r 2} {c}_{2} + {k}_{ri 2 a} {c}_{i 2 a} - {k}_{fi 2 a} {i}_{1} {c}_{1} + {k}_{ri 2 b} {c}_{i 2 b} - {k}_{fi 2 b} {i}_{2} {c}_{1} \\ {\dot{s}}_{2} = {k}_{r 2} {c}_{2} - {k}_{f 2} {s}_{2} {c}_{1} \\ {\dot{c}}_{2} = {k}_{f 2} {s}_{2} {c}_{1} - {k}_{r 2} {c}_{2} - {k}_{c 1} {c}_{2} \\ {\dot{c}}_{3} = {k}_{c 1} {c}_{2} + {k}_{r 3} {p}_{1} {c}_{4} - {k}_{f 3} {c}_{3} \\ {\dot{p}}_{1} = {k}_{f 3} {c}_{3} - {k}_{r 3} {p}_{1} {c}_{4} \\ {\dot{c}}_{4} = {k}_{f 3} {c}_{3} - {k}_{r 3} {p}_{1} {c}_{4} + {k}_{r 4} {p}_{2} {c}_{5} - {k}_{f 4} {c}_{4} \\ {\dot{p}}_{2} = {k}_{f 4} {c}_{4} - {k}_{r 4} {p}_{2} {c}_{5} \\ {\dot{c}}_{5} = {k}_{f 4} {c}_{4} - {k}_{r 4} {p}_{2} {c}_{5} - {k}_{f 5} {c}_{5} + {k}_{r 5} {c}_{6} {p}_{3} \\ {\dot{p}}_{3} = {k}_{f 5} {c}_{4} - {k}_{r 5} {p}_{3} {c}_{6} \\ {\dot{c}}_{6} = {k}_{f 5} {c}_{4} - {k}_{r 5} {p}_{3} {c}_{6} - {k}_{f 6} {c}_{6} + {k}_{r 6} {p}_{4} e \\ {\dot{p}}_{4} = {k}_{f 6} {c}_{6} - {k}_{r 6} {p}_{4} e \\ {\dot{c}}_{i 1 a} = {k}_{fi 1 a} {i}_{1} e - {k}_{ri 1 a} {c}_{i 1 a} - {k}_{fta} {s}_{1} {c}_{i 1 a} + {k}_{rta} {c}_{i 2 a} \\ {\dot{c}}_{i 1 b} = {k}_{fi 1 b} {i}_{2} e - {k}_{ri 1 b} {c}_{i 1 b} - {k}_{ftb} {s}_{1} {c}_{i 1 b} + {k}_{rtb} {c}_{i 2 b} \\ {\dot{c}}_{i 2 a} = {k}_{fta} {s}_{1} {c}_{i 1 a} - {k}_{rta} {c}_{i 2 a} + {k}_{fi 2 a} {i}_{1} {c}_{1} - {k}_{ri 2 a} {c}_{i 2 a} \\ {\dot{c}}_{i 2 b} = {k}_{ftb} {s}_{1} {c}_{i 1 b} - {k}_{rtb} {c}_{i 2 b} + {k}_{fi 2 b} {i}_{2} {c}_{1} - {k}_{ri 2 b} {c}_{i 2 b} \\ {\dot{i}}_{1} = {k}_{ri 1 a} {c}_{i 1 a} - {k}_{fi 1 a} {i}_{1} e + {k}_{ri 2 a} {c}_{i 2 a} - {k}_{fi 2 a} {i}_{1} {c}_{1} \\ {\dot{i}}_{2} = {k}_{ri 1 b} {c}_{i 1 b} - {k}_{fi 1 b} {i}_{2} e + {k}_{ri 2 b} {c}_{i 2 b} - {k}_{fi 2 b} {i}_{2} {c}_{1}\end{matrix}$$

Mixed Inhibition has been shown for mevalonate and phosphomevalonate with respect to ATP in the Gallus gallus homolog of the enzyme.

This may be actually competitive inhibition because dual mixed inhibition results in some nasty equations.

$${\dot{s}}_{1} = - \frac{{K}_{1} {e}_{0} s}{{K}_{2} {i}_{1} + {K}_{3} {i}_{2} + {K}_{4} s + {K}_{5}}$$

$${\dot{p}}_{1} = \frac{{K}_{1} {e}_{0} s}{{K}_{2} {i}_{1} + {K}_{3} {i}_{2} + {K}_{4} s + {K}_{5}}$$

Reaction 7

Isopentenyl diphosphate isomerase (IDI) mechanism with irreversible inhibition is shown below.

$$Isopentenyl diphosphate {\to }^{IDI} Dimethylallyl diphosphate$$

$${s}_{1} + e {{⇌}_{{k}_{r 1}}}^{{k}_{f 1}} e {\to }^{{k}_{c 1}} e + p$$

$${R}_{7} \{ \begin{matrix}\dot{s} = {k}_{r 1} c - {k}_{f 1} se \\ \dot{e} = {k}_{r 1} c - {k}_{f 1} se + {k}_{c 1} c \\ \dot{c} = {k}_{f 1} se - {k}_{r 1} c - {k}_{c 1} c \\ \dot{p} = {k}_{c 1} c\end{matrix}$$

### Briggs-Haldane Kinetics:

$$\dot{s} = - \frac{{K}_{cat} {e}_{0} s}{{K}_{d} + s}$$

$$\dot{p} = \frac{{K}_{cat} {e}_{0} s}{{K}_{d} + s} ,$$

$$where$$

$${K}_{cat} = {k}_{c 1}$$

$${K}_{d} = \frac{{k}_{c 1} + {k}_{r 1}}{{k}_{f 1}}$$

Reaction 8

The geranyl diphosphate synthase (GPPS) mechanism is shown below.

$$dimethylallyl diphosphate + isopentenyl diphosephate {\to }^{GPPS} diphosphate + geranyl diphosphate$$

$${s}_{1} + e {{⇌}_{{k}_{r 1}}}^{{k}_{f 1}} {c}_{1} + {s}_{2} {{⇌}_{{k}_{r 2}}}^{{k}_{f 2}} {c}_{2} {\to }^{{k}_{r 1}} {c}_{3} {{⇌}_{{k}_{r 3}}}^{{k}_{f 3}} {c}_{4} + {p}_{1} {{⇌}_{{k}_{r 4}}}^{{k}_{f 4}} e + {p}_{2}$$

$${R}_{8} \{ \begin{matrix}{\dot{s}}_{1} = {k}_{r 1} {c}_{1} - {k}_{f 1} {s}_{1} e \\ \dot{e} = {k}_{r 1} {c}_{1} - {k}_{f 1} {s}_{1} e + {k}_{f 4} {c}_{4} - {k}_{r 4} {p}_{2} e \\ {\dot{c}}_{1} = {k}_{f 1} {s}_{1} e - {k}_{r 1} {c}_{1} - {k}_{f 2} {s}_{2} {c}_{1} + {k}_{r 2} {c}_{2} \\ {\dot{s}}_{2} = {k}_{r 2} {c}_{2} - {k}_{f 2} {s}_{2} {c}_{1} \\ {\dot{c}}_{2} = {k}_{f 2} {s}_{2} {c}_{1} - {k}_{r 2} {c}_{2} - {k}_{c 1} {c}_{2} \\ {\dot{c}}_{3} = {k}_{c 1} {c}_{2} + {k}_{r 3} {p}_{1} {c}_{4} - {k}_{f 3} {c}_{3} \\ {\dot{c}}_{4} = {k}_{f 3} {c}_{3} - {k}_{r 3} {p}_{1} {c}_{4} + {k}_{r 4} {p}_{2} e - {k}_{f 4} {c}_{4} \\ {\dot{p}}_{1} = {k}_{f 3} {c}_{3} - {k}_{r 3} {p}_{1} {c}_{4} \\ {\dot{p}}_{2} = {k}_{f 4} {c}_{4} - {k}_{r 4} {p}_{2} e\end{matrix}$$

### Briggs-Haldane Kinetics:

$${\dot{s}}_{1} = - \frac{{K}_{1} {e}_{0} {s}_{1} {s}_{2}}{{K}_{2} + {K}_{3} {s}_{1} {K}_{4} {s}_{2} + {s}_{1} {s}_{2}}$$

$${\dot{s}}_{2} = - \frac{{K}_{1} {e}_{0} {s}_{1} {s}_{2}}{{K}_{2} + {K}_{3} {s}_{1} {K}_{4} {s}_{2} + {s}_{1} {s}_{2}}$$

$$\dot{p} = \frac{{K}_{1} {e}_{0} {s}_{1} {s}_{2}}{{K}_{2} + {K}_{3} {s}_{1} {K}_{4} {s}_{2} + {s}_{1} {s}_{2}} .$$

Reaction 9

Limonene Synthase finally makes limonene.

$$geranyl diphosphate {\to }^{LS} limonene + diphosphate$$

$${s}_{1} + e {{⇌}_{{k}_{r 1}}}^{{k}_{f 1}} {c}_{1} {\to }^{{k}_{c 1}} {c}_{2} {{⇌}_{{k}_{r 2}}}^{{k}_{f 2}} {c}_{3} + {p}_{1} {{⇌}_{{k}_{r 3}}}^{{k}_{f 3}} e + {p}_{2}$$

$${R}_{9} \{ \begin{matrix}\dot{s} = {k}_{r 1} {c}_{1} - {k}_{f 1} se \\ \dot{e} = {k}_{r 1} {c}_{1} - {k}_{f 1} se + {k}_{f 3} {c}_{3} - {k}_{r 3} {p}_{2} e \\ {\dot{c}}_{1} = {k}_{f 1} se - {k}_{r 1} {c}_{1} - {k}_{c 1} {c}_{1} \\ {\dot{c}}_{2} = {k}_{r 2} {p}_{1} {c}_{3} - {k}_{f 2} {c}_{2} - {k}_{c 1} {c}_{1} \\ {\dot{c}}_{3} = {k}_{f 2} {c}_{2} + {k}_{r 3} {p}_{2} e - {k}_{f 3} {c}_{3} \\ {\dot{p}}_{1} = {k}_{f 2} {c}_{3} - {k}_{r 2} {p}_{1} {c}_{3} \\ {\dot{p}}_{2} = {k}_{f 3} {c}_{3} - {k}_{r 3} {p}_{2} e\end{matrix}$$

### Briggs-Haldane Kinetics:

$$\dot{s} = - \frac{{K}_{1} {e}_{0} {k}_{f 3} s}{{K}_{1} s + {K}_{2} {p}_{2} + {K}_{3} {p}_{1} s + {K}_{4} {p}_{1} {p}_{2} + {K}_{5} {p}_{1} {p}_{2} + {K}_{6} s + {K}_{7}}$$

$${\dot{p}}_{1} = \frac{{e}_{0} {k}_{f 2} ( {K}_{1} s + {K}_{2} {p}_{2} - {K}_{3} {p}_{1} s - {K}_{4} {p}_{1} {p}_{2} - {K}_{5} {p}_{1} {p}_{2} )}{{K}_{1} s + {K}_{2} {p}_{2} + {K}_{3} {p}_{1} s + {K}_{4} {p}_{1} {p}_{2} + {K}_{5} {p}_{1} {p}_{2} + {K}_{6} s + {K}_{7}}$$

$${\dot{p}}_{2} = \frac{{K}_{1} {e}_{0} {k}_{f 3} s}{{K}_{1} s + {K}_{2} {p}_{2} + {K}_{3} {p}_{1} s + {K}_{4} {p}_{1} {p}_{2} + {K}_{5} {p}_{1} {p}_{2} + {K}_{6} s + {K}_{7}}$$

$${K}_{1} = {k}_{c 1} {k}_{f 1} {k}_{f 2}$$

$${K}_{2} = {k}_{c 1} {k}_{f 2} {k}_{r 3} + {k}_{f 2} {k}_{r 1} {k}_{r 3}$$

$${K}_{3} = {k}_{c 1} {k}_{f 1} {k}_{r 2}$$

$${K}_{4} = {k}_{c 1} {k}_{r 2} {k}_{r 3}$$

$${K}_{5} = {k}_{r 1} {k}_{r 2} {k}_{r 3}$$

$${K}_{6} = {k}_{c 1} {k}_{f 1} {k}_{f 3} + {k}_{f 1} {k}_{f 2} {k}_{f 3}$$

$${K}_{7} = {k}_{c 1} {k}_{f 2} {k}_{f 3} + {k}_{f 2} {k}_{f 3} {k}_{r 1} .$$

### Composite Model

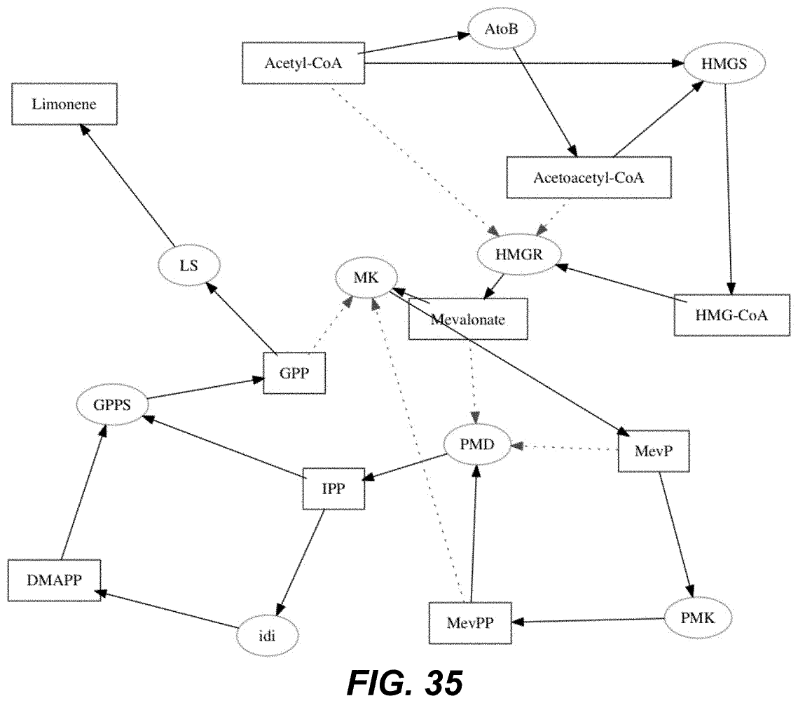

The complete set of reactions and inhibition relationships are given shown in FIG. 35. The Metabolites are inside of rectangles, the enzymes are in circles. Solid arrows indicate forward flow into the next component. Dashed arrows indicate an inhibition relationship between the two species.

### Reduced Order Michaelis-Menten Kinetics

Using the relationships derived above, a complete Michaelis-Menten description of the system is shown below.

$$\frac{d [ A - CoA ]}{dt} = - \frac{{K}_{1 , 1} [ AtoB ] [ A - CoA ]}{{K}_{1 , 2} + {K}_{1 , 3} [ A - CoA ]} - \frac{{K}_{2 , 1} [ HMGS ] [ A - CoA ] [ AA - CoA ] {k}_{s 3}}{{K}_{2 , 2} [ AA - CoA ] + {K}_{2 , 3} [ A - CoA ] + {K}_{2 , 4} [ A - CoA ] [ AA - CoA ]} \frac{d [ AA - CoA ]}{dt} = \frac{{K}_{1 , 1} [ AtoB ] [ A - CoA ]}{{K}_{1 , 2} {K}_{1 , 3} [ A - CoA ]} - \frac{{K}_{2 , 1} [ HMGS ] [ A - CoA ] [ AA - CoA ] {k}_{s 3}}{{K}_{2 , 2} [ AA - CoA ] + {K}_{2 , 3} [ A - CoA ] + {K}_{2 , 4} [ A - CoA ] [ AA - CoA ]} \frac{d [ HMG - CoA ]}{dt} = \frac{{K}_{2 , 1} [ HMGS ] [ A - CoA ] [ AA - CoA ] {k}_{s 3}}{{K}_{2 , 2} [ AA - CoA ] + {K}_{2 , 3} [ A - CoA ] + {K}_{2 , 4} [ A - CoA ] [ AA - CoA ]} - \frac{{K}_{3 , 1} [ HMGR ] [ HMG - CoA ]}{{K}_{3 , 2} [ A - CoA ] + {K}_{3 , 3} [ AA - CoA ] + {K}_{3 , 4} [ HMG - CoA ] + {K}_{3 , 5}} \frac{d [ Mev ]}{dt} = \frac{{K}_{3 , 1} [ HMGR ] [ HMG - CoA ]}{{K}_{3 , 2} [ A - CoA ] + {K}_{3 , 3} [ AA - CoA ] + {K}_{3 , 4} [ HMG - CoA ] + {K}_{3 , 5}} - \frac{{K}_{4 , 1} [ MK ] [ Mev ]}{{K}_{4 , 2} [ GPP ] + {K}_{4 , 3} [ MevP ] + {K}_{4 , 4} [ Mev ] + {K}_{4 , 5}} \frac{d [ MevP ]}{dt} = \frac{{K}_{4 , 1} [ MK ] [ Mev ]}{{K}_{4 , 2} [ GPP ] + {K}_{4 , 3} [ MevP ] + {K}_{4 , 4} [ Mev ] + {K}_{4 , 5}} - \frac{K 5 , 1 [ PMK ] [ MevP ]}{{K}_{5 , 1} + [ MevP ]} \frac{d [ MevPP ]}{dt} = \frac{K 5 , 1 [ PMK ] [ MevP ]}{{K}_{5 , 1} + [ MevP ]} - \frac{{K}_{6 , 1} [ PMD ] [ MevPP ]}{{K}_{6 , 2} [ MevP ] + {K}_{6 , 3} [ Mev ] + {K}_{6 , 4} [ MevPP ] + {K}_{6 , 5}} \frac{d [ IPP ]}{dt} = \frac{{K}_{6 , 1} [ PMD ] [ MevPP ]}{{K}_{6 , 2} [ MevP ] + {K}_{6 , 3} [ Mev ] + {K}_{6 , 4} [ MevPP ] + {K}_{6 , 5}} - \frac{{K}_{7 , 1} [ IDI ] [ IPP ]}{{K}_{7 , 2} + [ IPP ]} - \frac{{K}_{8 , 1} [ GPPS ] [ IPP ] [ DMAPP ]}{{K}_{8 , 2} + {K}_{8 , 3} [ IPP ] {K}_{8 , 4} [ DMAPP ] + [ IPP ] [ DMAPP ]} \frac{d [ DMAPP ]}{dt} = \frac{{K}_{7 , 1} [ IDI ] [ IPP ]}{{K}_{7 , 2} + [ IPP ]} - \frac{{K}_{8 , 1} [ GPPS ] [ IPP ] [ DMAPP ]}{{K}_{8 , 2} + {K}_{8 , 3} [ IPP ] + {K}_{8 , 4} [ DMAPP ] + [ IPP ] [ DMAPP ]} \frac{d [ GPP ]}{dt} = \frac{{K}_{8 , 1} [ GPPS ] [ IPP ] [ DMAPP ]}{{K}_{8 , 2} + {K}_{8 , 3} [ IPP ] {K}_{8 , 4} [ DMAPP ] + [ IPP ] [ DMAPP ]} - \frac{{K}_{9 , 1} [ LS ] [ GPP ]}{{K}_{9 , 2} + [ GPP ]} \frac{d [ Limonene ]}{dt} = \frac{{K}_{9 , 1} [ LS ] [ GPP ]}{{K}_{9 , 2} + [ GPP ]}$$

### Alternative Embodiments

In one embodiment, data on all relevant metabolites of interest is available. The system may have no unmeasured memory states. So, only data on the previous time point can be used to predict the next state. In one embodiment, models can be trained using partial knowledge of the state and a larger time series. Accordingly, fewer measurements may be used to accomplish the same dynamical estimation.

In one embodiment, the measurement of the entire state and its derivative at every time point can be noisy. These measurements may be difficult to acquire for the entire metabolism. In cases where the entire state cannot be measured, the methods disclosed herein can predict the derivatives of the measured quantities from a limited time history of the measurements taken. Modern deep learning techniques, such as long short term memory recurrent neural nets, can be implemented. The machine learning methods implemented can affect the number of strains for training effective models for modeling metabolic systems.

In one implementation, other supervised learning techniques may be used to improve predictions. For example, tree-based pipeline optimization tool (TPOT) may be used to combine, through genetic algorithms or processes, 11 different machine learning regressors and 18 different preprocessing (feature selection) methods. Additional supervised learning techniques may be included in this approach by adding them to the scikit-learn library. For example, TPOT may automatically test them and use them if they provide more accurate predictions than the techniques used here. Other methods for ML include deep-learning (DL) techniques based on neural networks. Data for training a DL-based model for learning and predicting metabolic pathway dynamics may be obtained. For example, data for more than 1000 strains may be obtained

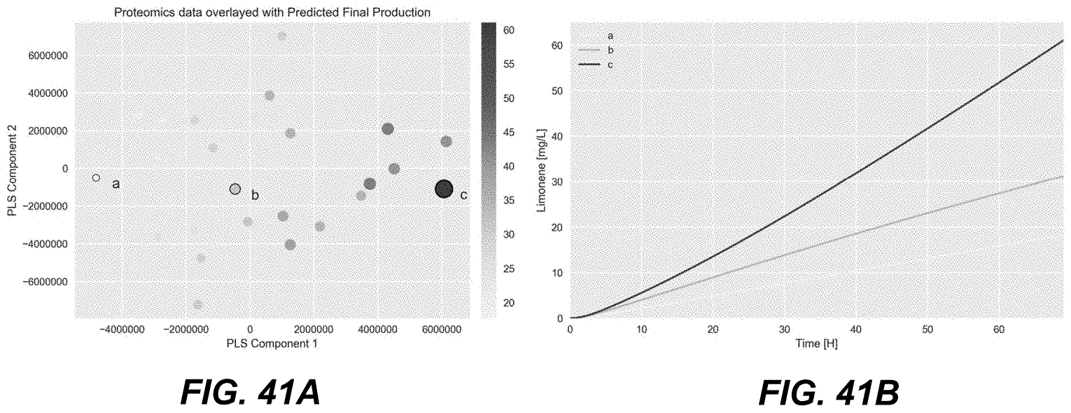

Mechanistic insights may be inferred from ML approaches disclosed herein. Exemplary possibilities for this inference include: (1) for any particular ML model that produces good fits, the most relevant features, such as protein x has the highest weight in determining y molecule concentration, provides a prioritized list of putative mechanistically linked parts that can be further investigated. (2) the ML model can be used as a surrogate for high-throughput experiments to derive mechanistic biological insights (FIGS. 41A-41B). Another example of this approach involves studying toxicity by adding cell biomass (through optical density (OD)) to the measurements and simulate for a variety of scenarios (protein inputs) the correlation between OD and all metabolites: a negative correlation would signal putative toxic metabolites.

The methods can include incorporating prior knowledge into the ML approach. In one implementation, the method constrains the vector fields that are learned using any biological intuition. Biological facts may be known about these dynamical systems that could be used to improve the performance of the methods. For example, genome-scale stoichiometric constraints could provide guarantees that the resulting system dynamics conserve mass and conform to prior knowledge about the organism.

The ML-based methods of the disclosure may only require little prior biological knowledge and may be extended for use with different data inputs or other types of applications. For example, transcriptomics data may be used as input. Given the current exponential increase in sequencing capabilities, transcriptomics data may be more amenable to high-throughput production than proteomics and metabolomics data. Transcriptomics data correlate with proteomics, and the methods may require more time-series data for accurate predictions. As another example, the ML method may be used to predict proteomics in addition to metabolomics time series. The input and output of the ML method may include genome-scale multiomics data. The genome-scale multiomics data may be dense.

In one implementation, the predictive capabilities of the machine learning method of with respect to the Michaelis-Menten approach proceed, in part, from indirectly accounting for host metabolism effects through proxies, such as metabolites or proteins that are affected indirectly by host metabolism. Hence, more comprehensive metabolomics and proteomics (as well as transcriptomics) data sets may increase the method predictive accuracy. The methods may be used to predict microbial community dynamics, as compared to intracellular pathway prediction, using meta-proteomics and metabolite concentration data as inputs.

### Determining Kinetic Models Using Meta Learning

This example demonstrates determining kinetic models using meta learning from time-series data using formulation I above.

The supervised learning method described above (FIGS. 28 and 29, Eqs. (1), (2), (3) and (4)) under Formulation I were used to predict pathway dynamics (i.e., metabolite concentrations as a function of time) from protein concentration data for two pathways of relevance to metabolic engineering and synthetic biology: a limonene producing pathway and an isopentenol producing pathway (FIG. 31). For each pathway, experimental times-series data obtained from the low and high biofuel producing strains were used as training data sets for a ML model, which was the used to predict the dynamics for the medium producing strains. TPOT was used to select the best pipelines it can find from the scikit-learn library combining 11 different regressors and 18 different preprocessing methods. This model selection process was done independently for each metabolite (Table 1). After TPOT determines the optimal models associated with each metabolite, the models were trained on the data set of interest and are ready for use to solve Eqs. (3) and (4). Models with the lowest tenfold cross-validated prediction root mean squared error was selected. In this way, the best validated models were selected for use. Because of the paucity of dense multiomics time-series data sets, simulated data sets were used (FIG. 34) to study the algorithm's performance as more training data sets (strains) were added.

TABLE 1
Table containing which machine learning model pipeline was used for each metabolite
derivative prediction along with a measure of each models' performance.

| | | | Fit Quality |
|---|---|---|---|
| Pathway | Metabolite | Machine Learning Model | (R Value) |
| Experimental | Acetyl-CoA | Extra Trees Regressor with | 1.000 |
| Isopentenol | | Polynomial Features | |
| | HMG-CoA | Lasso Lars CV | 0.993 |
| | | →Min Max Scaler | |
| | | →Gradient Boosting Regressor | |
| | | →Decision Tree Regressor | |
| | Mevalonate | Extra Trees Regressor | 1.000 |
| | Mev-P | FastICA | 1.000 |
| | | →LinearSVR | |
| | | →Extra Trees Regressor | |
| | IPP/DMAPP | Extra Trees Regressor | 1.000 |
| | Isopentenol | RidgeCV | 1.000 |
| | | →Extra Trees Regressor | |
| Experimental | Acetyl-CoA | FastICA | 0.996 |
| Limonene | | →Polynomial Features | |
| | | →Decision Tree Regressor | |
| | | →FastICS | |
| | | →Lasso-LarsCV | |
| | HMG-CoA | FastICA | 0.944 |
| | | →One Hot Encoder | |
| | | →Polynomial Features | |
| | | →Max Abs Scaler | |
| | | →K-Neighbors Regressor | |
| | Mevalonate | Variance Threshold | 1.000 |
| | | → RidgeCV | |
| | | →Min Max Scaler | |
| | | →K-Neighbors Regressor | |
| | Mev-P | Extra Trees Regressor | 0.994 |
| | | →Random Forest Regressor | |
| | | →Extra Trees Regressor | |
| | | →Decision Tree Regressor | |
| | IPP/DMAPP | Max Abs Scaler | 0.986 |
| | | →PCA | |
| | | →Max Abs Scaler | |
| | | →Max Abs Scaler | |
| | | → FastICA | |
| | | →Max Abs Scaler | |
| | | →RBFSampler | |
| | | → LassoLarsCV | |
| | Limonene | Extra Trees Regressor | 1.000 |
| | | → Random Forest Regressor | |
| Simulated | Acetyl-CoA | Random Forest Regressor with | 0.994 |
| Limonene | | Polynomial Features | |
| | Acetoacetyl-CoA | Random Forest Regressor | 0.997 |
| | HMG-CoA | Extra Trees Regressor | 1.000 |
| | Mevalonate | Extra Trees Regressor | 0.998 |
| | Mev-P | Min-Max Scaler | 0.997 |
| | | →Robust Scaler | |
| | | →Extra Trees Regressor | |
| | Mev-PP | PCA | 1.000 |
| | | → Extra Trees Regressor | |
| | IPP | Extra Trees Regressor | 0.997 |
| | DMAPP | Extra Trees Regressor | 1.000 |
| | | → LassoLarsCV | |
| | GPP | Fast ICA | 1.000 |
| | | →K-Neighbors Regressor | |
| | Limonene | K-Neighbors Regressor | 0.996 |

Qualitative Predictions of Limonene and Isopentenol Pathway Dynamics were Obtained with Two Time-Series Observations

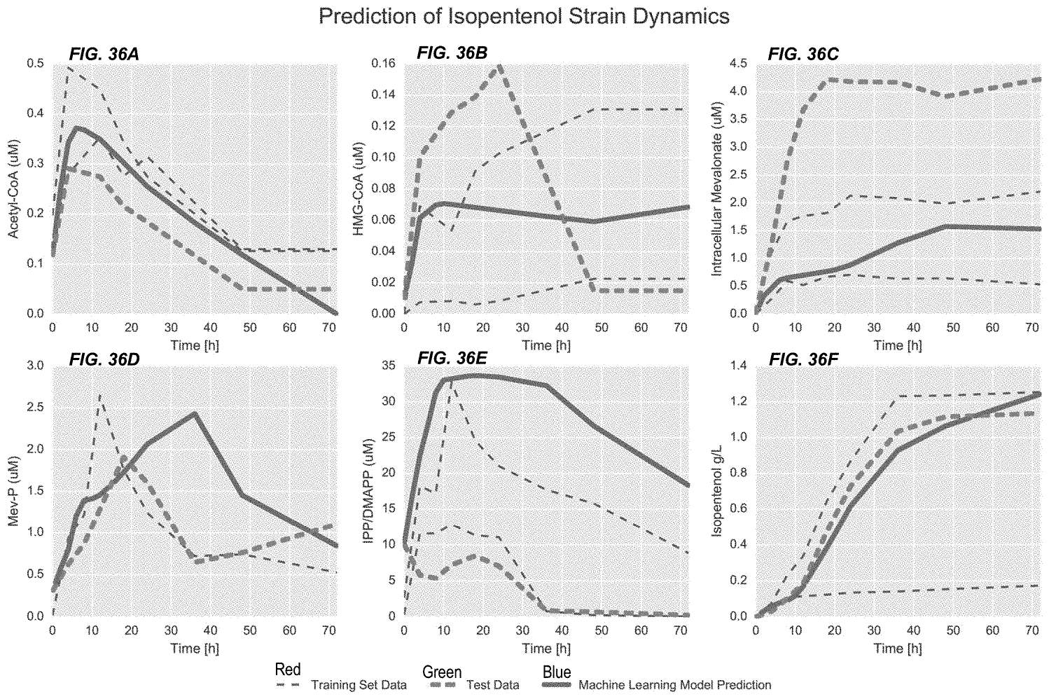

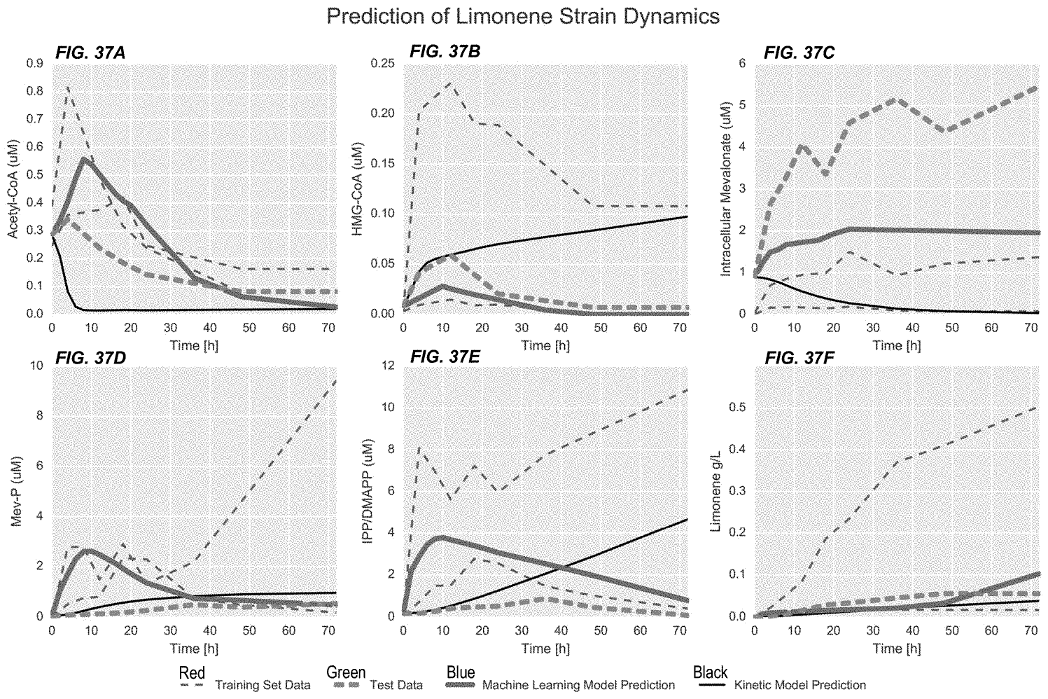

Two time-series (strains) were enough to train the ML model to produce acceptable predictions for most metabolites. The predictions of derivatives from proteomics and metabolomics were quite accurate (aggregate Pearson R value of 0.973), any small error in these predictions may compound quickly when solving the initial value problem given by Eqs. (3) and (4). For example, predictions for a given time point depend on the accuracy of all previous time points. The method produced respectable qualitative and quantitative predictions of metabolite concentrations for a strain it had never seen before (FIGS. 36A-36F and 37A-37F). For some metabolites (33%), the predictions were quantitatively close to the measured profile: acetyl-CoA (83.4% error, FIG. 36A) and isopentenol (43.7% error, FIG. 36F) for the isopentenol producing pathway; Acetyl-CoA (128.2% error, FIG. 37A), HMG-CoA (83.9% error, FIG. 37B) and limonene (82.9% error, FIG. 37F) for the limonene producing pathway. For most metabolites (42%), the predictions were off by a scale factor, but they were able to qualitatively reproduce the metabolite behavior. For example, for mevalonate in the isopentenol producing pathway (FIG. 36C) and mevalonate in the limonene producing pathway (FIG. 37C) the predictions reproduce the initial increase of metabolite concentration followed by a saturation. For IPP/DMAPP (FIG. 36E) or mevalonate phosphate (FIG. 36D) in the isopentenol pathway, the prediction reproduced qualitatively the concentration increase, followed by a peak and a decrease. The prediction of even just this type of qualitative behavior may be useful to metabolic engineers in order to obtain an intuitive understanding of the pathway dynamics and design better versions of it. By simulating several scenarios the metabolic engineer can extract qualitative knowledge (such as metabolite x seems toxic, or protein y seems regulated by metabolite x) that can lead to testable hypotheses. Finally, in a minority of cases (25%), the predictions required improvements both quantitatively and qualitatively: such as HMG-CoA for the isopentenol producing pathway (FIG. 36B), Mevalonate phosphate (FIG. 37D), and IPP/DMAPP (FIG. 37E) for the limonene producing pathway. The predictions for both final products (limonene and isopentenol) fell in the group of quantitatively accurate predictions. This may be important because, for the purpose of guiding metabolic engineering, it is the final product predictions that are relevant.

The machine learning approach outperformed a handcrafted kinetic model of the limonene pathway (FIGS. 37A-37F). A realistic kinetic model of this pathway was built and fit to the data, leaving all kinetic constants as free parameters (FIGS. 31 and 34). The kinetic model notably failed to capture the qualitative dynamics for Acetyl-CoA, HMG-CoA, mevalonate, and IPP/DMAPP (FIGS. 37A-37C, 37E). More quantitatively, the machine learning model produced an average 130% error (RMSE=8.42) vs. an average 144% (RMSE=10.04) for the kinetic model. Hence, even a machine learning model informed by the time-series data of just two strains was able to outperform the handcrafted kinetic model, which required domain expertise and significant time investment to construct. The machine learning approach, however, is more easily generalizable, and it can be reapplied for a new pathway, host or product by feeding it the corresponding data. Once the predictions were made for the limonene pathway, results for the isopentenol pathway can be obtained just by changing the time-series data input. In contrast, in order to make predictions for the isopentenol pathway a new kinetic model would have to be built. Kinetic models become more difficult to construct as the size of the reaction network increases and as the knowledge of the relevant network decreases. Additionally, all kinetic relationships must be known or inferred, whereas unknown relationships can be uncovered from data using a machine learning approach. The machine learning approach only requires a sufficient amount of data to disentangle these relationships.

The model was able to perform well even though the training sets corresponded to pathways which differed in more than just protein levels. This may be useful because the model was designed to take protein concentrations as input (FIG. 28) in order to predict pathway dynamics, assuming the rest of pathway characteristics to remain the same. The method can be applied to solve a wide range of metabolic engineering needs. For example, the model can be applied to promoters and ribosome-binding sites (RBSs) being modified in order to affect the resulting protein concentrations. As another example, the model can aid in designing metabolic engineering strategies, such as changing a given enzyme in order to access faster or slower catalytic rates (i.e., k_{cat}). The model was able to provide good predictions for 13, which used a HMGR analog form Staphylococcus aureus, and 12, which used a codon optimized HMGR. Without being limited by theories, k_{cat} changes may be renormalized into (and be equivalent to) protein abundance changes. In one implementation, this method may be expanded to include enzyme characteristics as input (besides the proteomics data): k_{cat} and Kₘ constants or even full kinetic characterization curves.

### Increasing the Number of Strains Improves the Accuracy of Dynamic Predictions

Simulated data was used to show that predictions improved markedly as more data sets are used for training. Simulated data sets had the advantage of providing unlimited samples to thoroughly test scaling behavior, and allowed a wider variety of types of dynamics than experimentally accessible to be explored. Moreover, the dense multiomics time-series data sets needed as training data may be rare because they are very time consuming and expensive to produce. Since machine learning predictions may improve as more data is used to train them, the method was expected to improve with the availability of more time series for training. This improvement was expected to be significant since initially only two time-series (strains) were used for training, out of the three available for each product (the other one was used for testing). Hence, simulated data obtained from using the kinetic model developed for the limonene pathway (FIGS. 31 and 34) was used to determine: (1) how much predictions improve as more time-series data sets are added and (2) how many time series are needed to guide pathway design effectively. A pool of 10,000 sets of time-series data with different protein profiles was created that shared the same kinetic constants. The pool of time-series data was fed the machine learning model in groups of 2, 10, and 100 times series randomly sampled from this pool in order to determine how quickly the model was able to recover the original simulated dynamics. In order to gauge the variability of the predictions (i.e., how predictions change as different training sets are used) as a function of training group size (2, 10, or 100), the predictions were repeated ten times for each training group size.

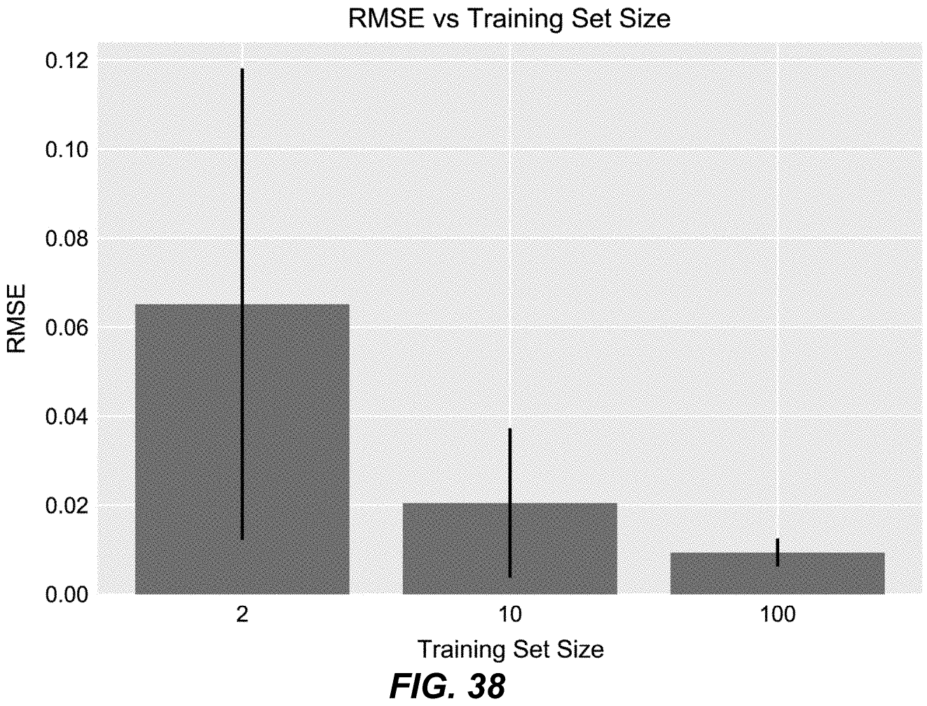

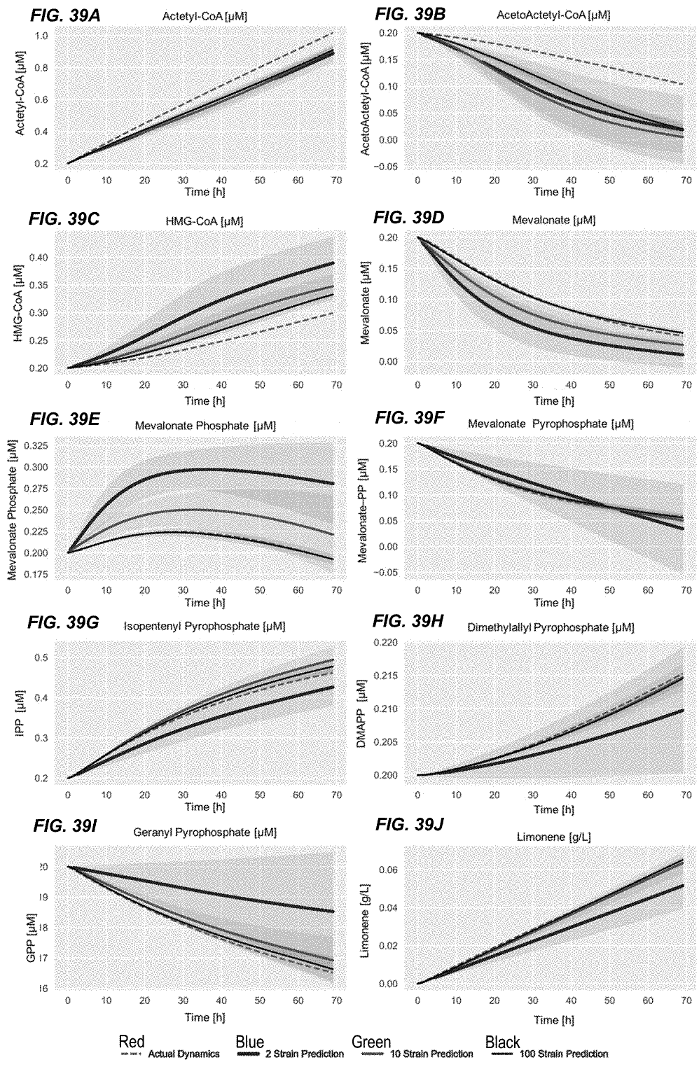

The prediction error (RMSE, Eq. (6)) decreased monotonically as a function of the number of time-series (strains) used to train the model in a nonlinear fashion (FIG. 38). Also, the standard deviation of the predictions significantly decreased with the number of training of data sets (FIGS. 39A-39J). The standard deviation is an indication of the variability of pathway dynamics predictions due to stochastic effects of the optimization processes (e.g., different seeds) and lack of extrapolability from a reduced set of initial protein concentrations. Hence, a predictive model trained with 10 or 100 data sets may produce more robust predictions than a model trained with two data sets. In fact, the high standard deviations observed for models trained on only two data sets may explain the prediction variability observed in the previous section due to stochastic effects. There was a limited drop in error and standard deviation from 10 to 100 strains, with the decrease from two to 10 being the largest (FIG. 38). This may indicate that it is more productive to do ten rounds of engineering collecting ten time-series data set than a single round collecting 100 time series: in this way, ten time series produce accurate enough predictions to pinpoint the desirable part of proteomics of phase space, new strains can be engineered around that space so that new multiomics time series can be obtained around the desirable phase space and optimize for prediction accuracy around that area of phase space. Doing this ten times may be more accurate than a single prediction based on 100 time series that may not be close to the ultimately desirable proteomics phase space. Furthermore, it indicates that the results from the previous section may have been much more reliable if only eight time series more had been available for training.

### Accurate Model Predictions for Guiding Pathway Design and Produce Biological Insights

The machine learning predictions may not need to be 100% quantitatively correct to accurately predict the relative ranking of production for different strains. Being able to reliably predict which of several possible pathway designs will produce the highest amount of product is very valuable in guiding bioengineering efforts and accelerating them in order to improve titer, rate, and yield (TRY). These process characteristics may be important determinants of economic relevance.

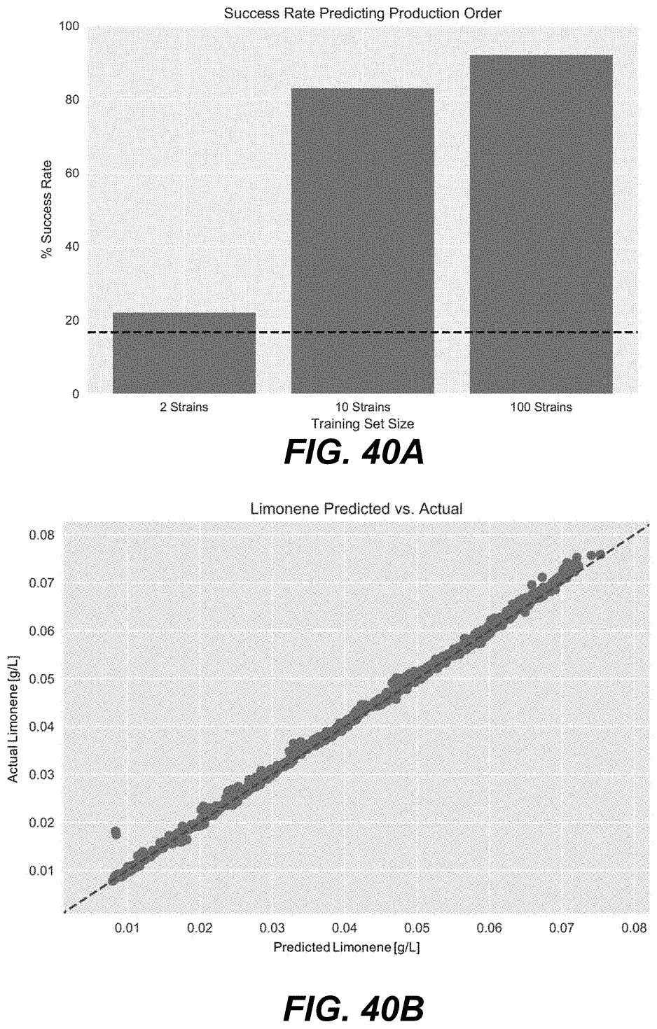

The machine learning model or process was able to reliably predict the relative production ranking for groups of three randomly chosen strains (highest, lowest, and medium producer, mimicking the available experimental data) chosen from the pool of 10,000 time-series data sets mentioned above (FIG. 40A). The success rate depended on the number of data sets available for training: starting at 22% for only two strains up to 92% for 100 training sets. For ten strains the success rate was ˜80%, which is reliable enough to practically guide metabolic engineer efforts to improve TRY. For models trained using 100 time series, the prediction errors were minimal (FIG. 40B).

Biological insights may be generated by using the machine learning (ML) model to produce data in substitution of bench experiments. For example, similarly to principal component analysis of proteomics (PCAP), the ML simulations may be used to determine which proteins to over or under express, and for which base strain, in order to improve production (FIGS. 41A and 41B). Proteins LS, AtoB, PMD, and Idi may be important drivers of production in the case of limonene: changing protein expression along the principal component associated with them increases limonene creation (FIG. 41A). Furthermore, this approach provided expected behavior for all metabolites in the pathway, and hypotheses can be made and tested experimentally (FIG. 41B).

To show how biological insights can be derived (FIGS. 41A and 41B), a ML model may be trained using a number of proteomics and metabolomics time series, using the Michaelis-Menten kinetic model as ground truth. For example, the number of proteomics and metabolomics time series may be 50. Additional proteomics time series may be held back as a test data set. For example, the number of metabolite time series used as a test data set may be 50. Each metabolite time series may be predicted using the machine learning model and the associated proteomic time series. The final time point proteomics and final production may be collected for each predicted strain. The final time point proteomics data may be plotted in two dimensions with a basis selected by performing a partial least squares (PLS) regression between the proteomics and final production data. These first basis vector from a PLS regression is the direction that explains the most covariance between the proteomics data and production data. The PLS regression was implemented by and used from scikit-learn.

TABLE 2
Basis Vectors of Partial Least Squares Regression. The first two components of the partial
least squares regression are shown. These components represent the line that explains the most
covariance in the dependent variable of final production.

| AtoB | HMGS | HMGR | MK | PMK | PMD | Idi | GPPS | LS |
|---|---|---|---|---|---|---|---|---|
| −0.375 | −0.098 | 0.006 | −0.191 | −0.242 | −0.372 | −0.312 | 0.021 | 0.719 |
| −0.018 | 0.426 | 0.504 | −0.274 | 0.446 | −0.259 | −0.422 | −0.078 | −0.193 |

### Data Constraints

Since the ML approach is data-based, data quantity and quality concerns are important. Data quantity concerns involve both the availability of enough time series as well as time points sampled in each time series.

The training set used in this example is one of the largest data sets characterizing a metabolically engineered pathway at regular time intervals through proteomics and metabolomics. There are no larger data sets that include: time series, several types of omics data, more than seven time points, and several strains. For example: the E. coli multiomics database has proteomics and metabolomics data for several strains, but no time series. For example, the database may include proteomics and metabolomics data but only one time series with fewer time points (five instead of seven); one time series and only one time point for proteomics; only time-series metabolomics data; metabolomics and proteomics data are not combined; genomics and not have any time-series proteomics or metabolomics; and any or minimal studies in terms of data points and strains.

In order to get enough pairs of derivatives and proteomics and metabolomics data to train ML models (FIG. 30), data augmentation (filtering and interpolation, FIG. 29 and FIG. 32) was used, expanding the initial seven time points to 200 by assuming continuity in the multiomics data (a reasonable assumption). It would be desirable to have more time points available, so as to not to depend on these data augmentation techniques. However, data sets including more time points were nonexistent for physical, biological, and economical reasons. Every time a sample is taken for omics analysis, the volume in the culture flask diminishes and, if the total sampled volume is comparable to the total volume, it may significantly affect the strain physiology. Since taking excessive samples may affect measurements, and these coupled omics analysis are expensive and require specialized personal, the maximum amount of time points was approximately seven. Another reason more time points have not been typically collected is that experts in multiomics data collection consider this sampling rate to fully capture the physiology of strains based on previous experience. The fact that it was possible to produce reasonable predictions for a third time series that the model has never seen before (test strain) validates this.

These results show that a data-centric approach to predicting metabolism that can greatly benefit the biotechnology and synthetic biology industries to enable reliable production. This approach is agnostic as to the pathway, host or product used, and can be systematically applied. This example also shows that, given sufficient data, the dynamics of complex coupled nonlinear systems relevant to metabolic engineering can be systematically learned.

### Execution Environment

FIG. 42 depicts a general architecture of an example computing device 4200 that can be used in some embodiments to execute the processes and implement the features described herein. The general architecture of the computing device 4200 depicted in FIG. 42 includes an arrangement of computer hardware and software components. The computing device 4200 may include many more (or fewer) elements than those shown in FIG. 42. It is not necessary, however, that all of these generally conventional elements be shown in order to provide an enabling disclosure. As illustrated, the computing device 4200 includes a processing unit 4210, a network interface 4220, a computer readable medium drive 4230, an input/output device interface 4240, a display 4250, and an input device 4260, all of which may communicate with one another by way of a communication bus. The network interface 4220 may provide connectivity to one or more networks or computing systems. The processing unit 4210 may thus receive information and instructions from other computing systems or services via a network. The processing unit 4210 may also communicate to and from memory 4270 and further provide output information for an optional display 4250 via the input/output device interface 4240. The input/output device interface 4240 may also accept input from the optional input device 4260, such as a keyboard, mouse, digital pen, microphone, touch screen, gesture recognition system, voice recognition system, gamepad, accelerometer, gyroscope, or other input device.

The memory 4270 may contain computer program instructions (grouped as modules or components in some embodiments) that the processing unit 4210 executes in order to implement one or more embodiments. The memory 4270 generally includes RAM, ROM and/or other persistent, auxiliary or non-transitory computer-readable media. The memory 4270 may store an operating system 4272 that provides computer program instructions for use by the processing unit 4210 in the general administration and operation of the computing device 4200. The memory 4270 may further include computer program instructions and other information for implementing aspects of the present disclosure.

For example, in one embodiment, the memory 4270 includes a kinetic learning module 4274 for training and/or using a machine learning model described herein, such as training a machine learning model and using the machine learning model to simulate a virtual strain of an organism or to determine possible modifications of an organism. In addition, memory 4270 may include or communicate with the data store 4290 and/or one or more other data stores for storage of multiomics data, a machine learning model trained using the multiomics data, and/or results (including intermediate results) of training and/or using a machine learning model.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1. A system for simulating a virtual strain of an organism, comprising:
computer-readable memory storing executable instructions and time-series multiomics data of an organism, wherein the times-series multiomics data comprises time-series proteomics data of a plurality of proteins and time-series metabolomics data comprising a characteristic of a metabolite; and
one or more hardware processors programmed by the executable instructions to perform:
training a machine learning model with the time-series proteomics data as input and the time-series metabolomics data of the metabolite as output; and
simulating a virtual strain of the organism using the machine learning model to determine the characteristic of the metabolite in the virtual strain.

2. The system of claim 1, wherein the time-series multiomics data comprises time-series multiomics data of a plurality of strains of the organism.

3. The system of claim 1, wherein the time-series proteomics data is associated with a metabolic pathway.

4. The system of claim 3, wherein the metabolic pathway comprises a heterologous pathway.

5. The system of claim 3, wherein the machine learning model represents kinetics of the metabolic pathway.

6. The system of claim 1, wherein the characteristic of the metabolite is a titer, rate, concentration, or yield of the metabolite.

7. The system of claim 1, wherein the proteomics data comprises a concentration of each of a plurality of proteins at each of a plurality of time points, and wherein the metabolomics data comprises a concentration of the metabolite at each of the plurality of time points.

8. The system of claim 1, wherein the multiomics data comprises triplicates of a concentration of a protein at a time point and triplicates of a concentration of the metabolite at a time point.

9. The system of claim 1, wherein simulating the virtual strain of the organism comprises determining a concentration of the metabolite of the virtual strain using the machine learning model.

10. The system of claim 1, wherein the machine learning model comprises a supervised machine learning model, a non-classification model, a neural network, a recurrent neural network (RNN), a linear regression model, a logistic regression model, a decision tree, a support vector machine, a Naïve Bayes network, a k-nearest neighbors (KNN) model, a k-means model, a random forest model, a multilayer perceptron, or a combination thereof.

11. (canceled)

12. The system of claim 1, wherein the machine learning model comprises a deep neural network (DNN), deep recurrent neural network (DRNN), gated recurrent unit (GRU) DRNN, a partial least square (PLS) model, or a combination thereof.

13. The system of claim 1, wherein the machine learning model comprises an ensemble model of a plurality of machine learning models, optionally wherein the plurality of machine learning models comprises a deep neural network (DNN), deep recurrent neural network (DRNN), and gated recurrent unit (GRU) DRNN.

14. The system of claim 1, wherein the virtual strain comprises an increased expression of at least one first protein, a knock-out of at least one second protein, a reduced expression of at least one third protein, or a combination thereof, optionally wherein the at least one first protein comprises at least 10 first proteins, optionally wherein the at least one second protein comprises at least 10 second proteins, optionally wherein the at least one third protein comprises at least 10 third proteins.

15. The system of claim 1, wherein the one or more hardware processors are further programmed to perform:
designing one or more new strains based on the virtual strain;
receiving experimental time-series multiomics data for the new strains; and
retraining the machine learning model based on the experimental time-series multiomics data of the new strains.

16. The system of claim 1, wherein the one or more hardware processors are further programmed to perform: interpolating the time-series multiomics data from a first number of time points to a second number of time points, optionally wherein the first number of time points comprises 8 time points, optionally wherein the second number of time points comprises 63 time points, optionally wherein the first number of time points are hourly time points, optionally wherein the second number of time points are hourly time points, and optionally wherein interpolating the time-series multiomics data comprises interpolating the time-series multiomics data using a cubic spline method.

17. A method for stimulating a strain of an organism, comprising:
receiving time-series multiomics data of a plurality of strains of an organism comprising time-series proteomics data of a plurality of proteins and time-series metabolomics data comprising a characteristic of a metabolite;
training a machine learning model with the time-series proteomics data as input and the time-series metabolomics data of the metabolite as output; and
simulating a virtual strain of the organism using the machine learning model to determine the characteristic of the metabolite in the virtual strain.

18. The method of claim 17, wherein receiving the time-series multiomics data comprises data checking and/or preprocessing of the time-series multiomics data of the plurality of strains of the organism.

19. The method of claim 17, wherein the time-series multiomics data comprises multiomics data of two or more time-series of a strain.

20.-25. (canceled)

26. The method of claim 17, further comprising designing a strain of the organism corresponding to the virtual strain and/or creating a strain of the organism corresponding to the virtual strain.

27. (canceled)

28. A method for determining modifications of protein expression an organism, comprising:
receiving time-series multiomics data of a plurality of strains of an organism comprising time-series proteomics data of comprising a characteristic of each of a plurality of proteins and time-series metabolomics data comprising a characteristic of a metabolite;
training a machine learning model with the time-series proteomics data as input and the time-series metabolomics data of the metabolite as output; and
determining modifications of a concentration of each of one or more proteins using the machine learning model.

29. (canceled)

30. (canceled)
